(19) United States
(12) Patent Application Publication
(10) Pub. No.: US 2026/0112455 A1
(43) Pub. Date: Apr. 23, 2026

(54) SYSTEMS AND METHODS FOR DISCOVERING COMPOUNDS USING INTERACTION FEATURES

(71) Applicant: DeepCure Inc., Boston, MA (US)

(72) Inventors: Jonathan Kaufman, Malden, MA (US); Derek Miller, Norfolk, MA (US)

(21) Appl. No.: 19/364,973

(22) Filed: Oct. 21, 2025

Related U.S. Application Data
(60) Provisional application No. 63/711,041, filed on Oct. 23, 2024.

(51) Int. Cl. G16C 10/00; G16C 20/50; G16C 20/70

(52) U.S. Cl. CPC G16C 10/00; G16C 20/50; G16C 20/70

(57) ABSTRACT

A method for identifying compounds with threshold activity against a target macromolecule first generates multiple poses for each compound fragment in a plurality of compound fragments against an atomic model of the target macromolecule. This creates a collection of configurations, or a “pose set,” for the compound fragments. Each pose is associated with a subset of interaction features drawn from a broader set of such features. Each feature corresponds to a subregion of the target macromolecule's atomic model. Each pose is quantified by application to a physics model. This assigns a score to the interaction features associated with the poses. A binding hypothesis is formed for the macromolecule, using the collection of interaction features and their corresponding scores. From this hypothesis, derived compounds are identified. These derived compounds are tested for their activity against the macromolecule, leading to the identification of those that exhibit the desired threshold activity.

### CROSS REFERENCE TO RELATED APPLICATIONS

This application claims priority to U.S. Provisional Patent Application No. 63/711,041 entitled “SYSTEMS AND METHODS FOR DISCOVERING COMPOUNDS USING INTERACTION FEATURES,” filed Oct. 23, 2024, which is hereby incorporated by reference.

### TECHNICAL FIELD

This application is directed to identifying compounds with threshold activity against macromolecule using scored interaction features.

### BACKGROUND

Pharmaceutical companies spend millions of dollars screening compounds to discover novel compounds and develop them into prospective drug leads. Traditionally, this has involved collecting large libraries of compounds tested to find the small number of compounds that interact with the disease target of interest. Unfortunately, gathering these large screening collections imposes significant challenges through storage constraints, shelf stability, or chemical cost. Furthermore, the cost and time needed to physically assay of compounds is prohibitive to testing them at scale. Even the largest pharmaceutical companies are testing only hundreds of thousands to a few millions of compounds at a time, versus the tens of millions of commercially available compounds and the billions, and even trillions of compounds that can be generated and screened computationally.

One key characteristic of a successful drug candidate is strong binding against its disease target. However, compounds that bind strongly enough to be clinically effective are rare.

Approximately half of the drug candidates in late-stage clinical trials fail due to unacceptable toxicity. Toxicity can be due to off-target side effects caused by a compound binding non-selectively to other targets. Therefore, increasing potent binding to the desired target while decreasing non-selective binding to other related targets is important in drug discovery. Drug candidates can also fail because they do not have desirable pharmacological absorption, distribution, metabolic, and excretion (ADME) profiles. Optimizing and balancing multiple objectives such as potency, selectivity, toxicity, and pharmacological properties is challenging but essential for a compound to become a drug.

Due to the many requirements for a compound to be a drug, there is a need to explore large and diverse chemical spaces of compounds that have different interactions with the target and, therefore, different properties. Large and diverse libraries of compounds also increase the odds of finding compounds that simultaneously satisfy all the other ADME properties needed to be a safe and effective drug. Thus, a better method is needed to accurately, rapidly, and efficiently identify or generate compounds that interact with the desired target.

Given the above background, what is needed in the art are methods for designing, identifying, and/or generating candidate compounds having target interaction properties when complexed with target macromolecules.

### SUMMARY

The present disclosure addresses the problems identified in the background by providing systems and methods that identify compounds with threshold activity against a target macromolecule by first generating multiple poses for each compound fragment in a plurality of compound fragments against an atomic model of the target macromolecule. This creates a collection of configurations, or a “pose set,” for the compound fragments. Each pose is associated with a subset of interaction features drawn from a broader set of such features. Each feature corresponds to a subregion of the target macromolecule's atomic model. Each pose is quantified by application to a physics model. This assigns a score to the interaction features associated with the poses. A binding hypothesis is formed for the macromolecule, using the collection of interaction features and their corresponding scores. From this hypothesis, derived compounds are identified. These derived compounds are tested for their activity against the macromolecule, leading to the identification of those that exhibit the desired threshold activity.

### I. Selecting Physics Weighted Interaction Features to Use in Compound Screening.

In more detail, one aspect of the present disclosure provides a method for identifying one or more compounds that exhibit a threshold activity with respect to a target macromolecule.

In some embodiments, the target macromolecule is a protein, a polypeptide, a polynucleic acid, a polyribonucleic acid, a polysaccharide, or an assembly of any combination thereof.

### A) Generating a Corresponding Plurality of Poses of Each Respective Compound Fragment in a Plurality of Compounds Fragments Against an Atomic Model of the Target Macromolecule Thereby Constructing a Pose Set.

There is generated, for each respective compound fragment in a plurality of compound fragments, a corresponding plurality of poses of the respective compound fragment against an atomic model of the target macromolecule thereby constructing a pose set.

In some embodiments, the atomic model of the target macromolecule is defined by a plurality of atomic coordinates of atoms of the plurality of residues derived by X-ray crystallography, neutron diffraction, cryo-electron microscopy, sampling from computational simulations, homology modeling, rotamer library sampling, or any combination thereof.

In some embodiments, the plurality of compound fragments comprises 1000 or more fragments, 5000 or more fragments, 10,000 or more fragments, 25,000 or more fragments, 50,000 or more fragments, 100,000 or more fragments, 1×106 or more fragments, or 1×107 or more fragments.

In some embodiments, each corresponding plurality of poses comprises 2 or more poses, 5 or more poses, 10 or more poses, 25 or more poses, or 50 or more poses.

In some embodiments, each corresponding plurality of poses comprises between 2 and 100 poses.

B) Associating a Corresponding Subset of Interaction Features, Drawn from a Plurality of Interaction Features, to Each Pose in the Pose Set.

A corresponding subset of interaction features, drawn from a plurality of interaction features, is associated with each pose in the pose set, wherein each interaction feature in the plurality of interaction features is associated with a corresponding subregion of the atomic model of the target macromolecule.

In some embodiments, the plurality of interaction features collectively identifies between 30 and 700 atoms of the target macromolecule.

In some embodiments, the plurality of interaction features collectively identifies between 50 and 500 atoms of the target macromolecule.

In some embodiments, the plurality of interaction features comprises a plurality of interaction feature types.

In some embodiments, the corresponding subregion of the atomic model comprises a portion of a surface of the atomic model of the target macromolecule.

In some embodiments, the atomic model comprises a plurality of residues and the corresponding subregion of the atomic model is a first residue in the plurality of residues or an atom of the first residue.

In some embodiments, the atomic model comprises a plurality of residues and the corresponding subregion of the atomic model is a subset of residues in the plurality of residues or a plurality of atoms of the first subset of residues.

In some embodiments, the pose set is clustered thereby assigning each pose in the pose set to a cluster in a plurality of clusters. The cluster assignment of each pose is used to filter the pose set.

In some embodiments, the clustering reduces a number of poses in the pose set by at least fifty percent, at least sixty percent, at least seventy percent, at least eighty percent, or at least ninety percent.

In some embodiments, the clustering of the pose set is based on a spatial overlap between poses.

### C) Quantifying Each Pose in the Plurality of Poses by Applying a Physics Model to Each the Pose, Thereby Assigning a Score to Each Interaction Feature in the Plurality of Interaction Features.

Each respective pose in the plurality of poses is quantified by applying a physics model to each respective pose in the pose set, thereby assigning a score to each interaction feature in the plurality of interaction features.

In some embodiments, the physics model is a first model comprising a first plurality of parameters. The quantifying comprises inputting the respective pose into the first model, and obtaining, through application of the respective pose to the first plurality of parameters in accordance with the first model, a pose score for the respective pose. The method further comprises using the pose score for each respective pose in the plurality of poses to determine a score for each interaction feature in the plurality of interaction features.

In some embodiments, the first plurality of parameters comprises at least 10,000, at least 100,000, or at least 1×10⁶ parameters.

In some embodiments, the physics model evaluates an interaction energy of the pose.

In some embodiments, the physics model evaluates the interaction energy of the pose using a calculated potential energy surface of the pose. The potential energy surface is calculated by the physics model using a molecular mechanics algorithm, a quantum mechanics algorithm.

In some embodiments, the physics model evaluates the pose against an interaction feature contract.

D) Forming a Target Macromolecule Binding Hypothesis Using the Plurality of Interaction Features and their Scores.

A target macromolecule binding hypothesis is formed using the plurality of interaction features and the score assigned to each interaction feature in the plurality of interaction features.

In some embodiments, a set of residues of the target macromolecule are identified that are included in the target macromolecule binding hypothesis.

In some embodiments, a subset of poses is selected from the plurality of poses that each has an interaction feature associated with a first residue in a plurality of residues of the atomic model. A first pose from the subset of poses that has the lowest energy score in the subset of poses is selected. One or more interaction features associated with the first pose is included in the target macromolecule binding hypothesis.

In some embodiments, a second pose is selected from the plurality of poses on the basis that it is associated with a second interaction feature that is other than any of the one or more interaction features associated with the first pose. The second interaction feature is included in the target macromolecule binding hypothesis.

In some embodiments, the target macromolecule binding hypothesis comprises the top N interaction features in the plurality of interaction features, where N is a positive integer.

In some embodiments, n is between 10 and 10,000 or N is at least 10, at least 25, at least 50, at least 100, or at least 500.

### E) Identifying a Plurality of Derived Compounds Based on the Target Macromolecule Binding Hypothesis.

A plurality of derived compounds is identified based on the target macromolecule binding hypothesis.

In some embodiments, the identifying comprises generating the plurality of derived compounds constrained to the target macromolecule binding hypothesis.

In some embodiments, the identifying comprises generating a plurality of initial compounds using the target macromolecule binding hypothesis and evolving at least a subset of the plurality of initial compounds into the plurality of derived compounds using a reinforcement learning process.

In some embodiments, the reinforcement learning process eliminates at least a subset of the plurality of initial compounds.

In some embodiments, the reinforcement learning process comprises: i) generating, using a computer, a plurality of experiences, each respective experience in the plurality of experiences using an initial compound selected from the plurality of initial compounds to construct a corresponding derived compound in the plurality of derived compounds through a hierarchical proximal policy comprising a parent model and a child model using an environment of the target macromolecule, wherein the parent model is a molecular reaction model that evaluates a plurality of molecular reactions to apply to an initial compound to form the derived compound, and the child model is a reactant model that evaluates a corresponding plurality of reactants for a molecular reaction applied to the initial compound to form the derived compound.

In some embodiments, the reinforcement learning process further comprises: ii) updating a second plurality of parameters associated with the parent model in accordance with a first surrogate objective calculated using the plurality of experiences, iii) updating a third plurality of parameters associated with a child model in accordance with a second surrogate objective using the plurality of experiences; iv) repeating, using a computer, the generating i), updating ii), and updating iii) until a threshold convergence criterion is satisfied.

In some embodiments, an experience in the plurality of experiences is generated by: (a) initializing the experience to state t=0, (b) inputting a complex of state t, in two or three dimensions, of the initial compound in state t interacting with the environment of the target macromolecule into the parent model. The parent model evaluates a first exit vector of the initial compound in state t against the plurality of molecular reactions, thereby assigning a corresponding probability to each respective molecular reaction in the plurality of molecular reactions for state t. (c) A molecular reaction in the plurality of molecular reactions is selected through a sampling of the plurality of molecular reactions using the corresponding probability assigned to each molecular reaction in the plurality of molecular reactions for state t. (d) The complex of state t is inputted into the child model. The child model evaluates the initial compound in state t against each reactant in a corresponding plurality of reactants available for reaction using the molecular reaction selected for state t, thereby assigning a corresponding probability to each respective reactant in the corresponding plurality of reactants for state t. (e) A reactant in the corresponding plurality of reactants is selected, through a sampling of the corresponding plurality of reactants using the corresponding probability assigned to each reactant in the corresponding plurality of reactants for state t, (f) State t is advanced to state t+1. (g) The initial compound in state t is formed through an in silico reaction of the initial compound in state t−1 in accordance with the selected molecular reaction and the selected reactant of state t, (h) A score is determined for the initial compound in state t interacting with the environment of the target macromolecule by inputting the initial compound in state t interacting with the environment of the target macromolecule into a physics model. The (b) inputting, (c) selecting, (d) inputting, (e) selecting, (f) advancing, (g) forming, and (h) determining is repeated until a compound exit criterion is satisfied by the initial compound in state t, thereby forming a plurality of states for the experience.

In some such embodiments, the identifying comprises in silico screening of a database of compounds using the on the target macromolecule binding hypothesis as a selection criterion.

F) Testing the Plurality of Derived Compounds for Activity Against the Target Macromolecule, Thereby Identifying One or More Compounds that Exhibit the Threshold Activity with Respect to the Target Macromolecule.

The plurality of derived compounds is tested for activity against the target macromolecule, thereby identifying one or more compounds that exhibit the threshold activity with respect to the target macromolecule.

In some embodiments, each compound in the one or more compounds is an organic compound having a molecular weight of less than 50 Daltons, less than 100 Daltons, less than 150 Daltons, less than 200 Daltons, less than 250 Daltons, less than 300 Daltons, less than 400 Daltons, less than 500 Daltons, or less than 1000 Daltons.

In some embodiments, each compound in the one or more compounds is an organic compound having a molecular weight of between 500 Daltons and 1000 Daltons.

In some embodiments, each compound in the one or more compounds is an organic compound having a molecular weight of between 400 Daltons and 10000 Daltons.

In some embodiments, each compound in the one or more compounds satisfies any two or more, any three or more, or all four of the conditions: (i) not more than five hydrogen bond donors, (ii) not more than ten hydrogen bond acceptors, (iii) a molecular weight under 500 Daltons, and (iv) a Log P under 5.

In some embodiments, the threshold activity with respect to the target macromolecule is an IC50, EC50, Kd, KI, hill coefficient (nH), negative logarithm of EC₅₀ (pEC50), association rate constant (Kon), or disassociation rate constant (Koff), for a compound with respect to the target macromolecule.

In some embodiments, the testing tests the plurality of derived compounds using a quantum mechanics algorithm.

In some embodiments, the testing tests the plurality of derived compounds using a wet lab assay.

Another aspect of the present disclosure provides a computer system for identifying one or more compounds that exhibit a threshold activity with respect to a target macromolecule, the computer system comprising one or more processors and memory addressable by the one or more processors. The memory stores at least one program for execution by the one or more processors. The at least one program comprises instructions for performing any of the methods disclosed herein.

Another aspect of the present disclosure provides a non-transitory computer readable storage medium. The non-transitory computer readable storage medium stores instructions, which when executed by a computer system, cause the computer system to perform a method of identifying one or more compounds that exhibit a threshold activity with respect to a target macromolecule, where the method comprises any of the methods disclosed herein.

### II. Using a Physics Model to Identify Compound Fragments as Derived Compounds for Further Testing.

Another aspect of the present disclosure provides a method for filtering a plurality of compound fragments to identify one or more compounds that exhibit a threshold activity with respect to a target macromolecule. The method comprises A) generating, for each respective compound fragment in a plurality of compound fragments, a corresponding plurality of poses of the respective compound fragment against an atomic model of the target macromolecule thereby constructing a pose set. The method further comprises B) associating, a corresponding subset of interaction features, drawn from a plurality of interaction features, to each pose in the pose set, wherein each interaction feature in the plurality of interaction features is associated with a corresponding subregion of the atomic model of the target macromolecule. The method further comprises C) selecting a subset of the poses from the pose set, wherein each pose set in the subset of pose sets is associated with at least one interaction feature in the plurality of interaction features. The method further comprises D) quantifying each pose in the subset of poses by applying a physics model to the pose using a neighborhood within the atomic model of the target macromolecule around the pose thereby forming a scored set of poses. The method further comprises E) identifying a set of top scored compound fragments from the scored set of poses. The method further comprises F) testing the plurality of top scored compound fragments for activity against the target macromolecule, thereby identifying one or more compounds that exhibit the threshold activity with respect to the target macromolecule.

### III. Using a Physics Model to Identify Compound Fragments for Reinforcement Learning.

Another aspect of the present disclosure provides a method for filtering a plurality of compound fragments to identify one or more compounds that exhibit a threshold activity with respect to a target macromolecule comprising a plurality of residues. The method comprises A) generating for each respective compound fragment in a plurality of compound fragments, a corresponding plurality of poses of the respective compound fragment against an atomic model of the target macromolecule thereby constructing a pose set. The method further comprises B) associating a corresponding subset of interaction features, drawn from a plurality of interaction features, to each pose in the pose set, wherein each interaction feature in the plurality of interaction features is associated with a corresponding subregion of the atomic model of the target macromolecule. The method further comprises C) selecting a subset of the poses from the pose set, wherein each pose set in the subset of pose sets is associated with at least one interaction feature in the plurality of interaction features. The method further comprises D) quantifying, using a computer, each pose in the subset of poses by applying a physics model to the pose using a neighborhood within the atomic model of the target macromolecule around the pose thereby forming a scored set of poses. The method further comprises E) identifying, using a computer, a set of top scored compound fragments from the scored set of poses. The method further comprises F) evolving at least a subset of the set of top scored compound fragments into a plurality of derived compounds using a reinforcement learning process. The method further comprises G) testing the plurality of plurality of derived compounds for activity against the target macromolecule, thereby identifying one or more compounds that exhibit the threshold activity with respect to the target macromolecule.

### BRIEF DESCRIPTION OF THE DRAWINGS

In the drawings, embodiments of the systems and methods of the present disclosure are illustrated by way of example. It is to be expressly understood that the description and drawings are only for the purpose of illustration and as an aid to understanding, and are not intended as a definition of the limits of the systems and methods of the present disclosure.

FIGS. 1A and 1B illustrate a computer system in accordance with some embodiments of the present disclosure.

FIGS. 2A, 2B, 2C, 2D, 2E, 2F, 2G, 2H, and 2I illustrate an example workflow for identifying one or more compounds that exhibit a threshold activity with respect to a target macromolecule, in accordance with some embodiments of the present disclosure.

FIG. 3 is a schematic view of an example workflow for using a target macromolecule binding hypothesis to evolve at least a subset of the plurality of initial compounds into a plurality of derived compounds using a reinforcement learning process, in accordance with some embodiments of the present disclosure.

FIGS. 4A, 4B, and 4C illustrate an example workflow for identifying one or more derived compounds that exhibit a threshold activity with respect to a target macromolecule, in accordance with some embodiments of the present disclosure.

FIG. 5 illustrates an initial compound at various states within an experience, culminating in a derived compound, in accordance with some embodiments of the present disclosure.

FIG. 6 illustrates a parent and child model using in accordance with an embodiment of the present disclosure.

FIG. 7 illustrates a stylized view of a target macromolecule with an environment that is a binding pocket in accordance with the prior art.

FIG. 8 illustrates poses of compound fragments that have been docked to an atomic model of signal transducer and activator of transcription 6 (STAT6) in accordance with an embodiment of the present disclosure.

FIG. 9 illustrates partial charge interactions of compound fragments that have been docked to an atomic model of STAT6 in accordance with an embodiment of the present disclosure.

FIG. 10 illustrates pharmacophores of compound fragments that have been docked to an atomic model of STAT6 in accordance with an embodiment of the present disclosure.

Like reference numerals refer to corresponding parts throughout the several views of the drawings.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

Drug discovery efforts often suffer from significant bottlenecks, including the ability to identify hit compounds and validate any such identified hit compounds as lead compounds for eventually synthesis and testing. These difficulties can be attributed, at least in part, to the massive size of custom molecule libraries that are searched in these early stages, which can reach up to 10¹² candidate molecules. Conventional methods, including traditional screening, fragment-based screening, and various machine learning and artificial intelligence pipelines, require laborious hit identification and/or hit-to-lead steps that increase the overall time, cost, and resource expenditure of drug discovery.

Advantageously, the systems and methods disclosed herein allow for rational design of molecules that meet stringent criteria imposed by target macromolecule binding hypotheses of the present disclosure. In particular, the systems and methods disclosed herein provide a unique platform that can be used to identify lead-like candidates.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

### Definitions

The terminology used in the present disclosure is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. It will also be understood that the term “and/of” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “target” refers to an object of interest, such as a macromolecule, macromolecule complex, or polymer that is of interest as a primary binding target for a candidate molecule. As used herein, the term “off-target” refers to an object that is not the primary binding target, such as a macromolecule, macromolecule complex, or polymer that exhibits off-target binding with a candidate molecule.

As used interchangeably herein, the terms “pose” or “conformation” refer to a pose of a compound when complexed to a target macromolecule. In some embodiments, a pose refers to the complex formed between a target macromolecule and any suitable compound capable of complexing to the target macromolecule including, but not limited to a initial compound, derived compound, a ligand, a reference molecule, a training molecule, a molecular component, and/or a molecular intermediate.

In some embodiments, a pose is determined by one or more docking programs. In some embodiments, one docking program is used to determine some of the poses for a complex between a compound and a target macromolecule and another docking program is used to determine other poses for the complex between the compound and the target macromolecule.

In some embodiments, one or more poses are determined using AutoDock Vina. See, Trott and Olson, “AutoDock Vina: improving the speed and accuracy of docking with a new scoring function, efficient optimization and multithreading,” Journal of Computational Chemistry 31 (2010) 455-461. In some embodiments, one or more poses are determined using Quick Vina 2 (Alhossary et al., 2015, “Fast, accurate, and reliable molecular docking with QuickVina,” Bioinformatics 31:13, pp. 2214-2216), VinaLC (Zhang et al., 2013, “Message Passing Interface and Multithreading Hybrid for Parallel Molecular Docking of Large Databases on Petascale High Performance Computing Machines,” J. Comput. Chem. DOI: 10.1002/jcc.23214), Smina (Koes et al., 2013, “Lessons learned in empirical scoring with smina from the CSAR 2011 benchmarking exercise,” Journal of chemical information and modeling 53:8, pp. 1893-1904), or CUina (Morrison et al., “Efficient GPU Implementation of AutoDock Vina,” COMP poster 3432389).

In some embodiments, one or more ensembled poses are determined using an ensembled docking algorithm such as disclosed in Stafford et al., 2022, “AtomNet PoseRanker: Enriching Ligand Pose Quality for Dynamic Proteins in Virtual High-Throughput Screens,” Journal of Chemical Information and Modeling 62, pp. 1178-1189, which is hereby incorporated by reference. In some such embodiments the ensemble consists of between 3 and 64, between 4 and 128, between 5 and 32, more than 5, or between 8 and 25 structurally similar poses.

In some embodiments, a compound is docked to a target macromolecule by either random pose generation techniques or by biased pose generation. In some embodiments, a compound is docked to a macromolecule by Markov chain Monte Carlo sampling. In some embodiments, such sampling allows the full flexibility of the compound in the docking calculations and a scoring function that is the sum of the interaction energy between the compound and the macromolecule as well as the conformational energy of the molecule. See, for example, Liu and Wang, 1999, “MCDOCK: A Monte Carlo simulation approach to the molecular docking problem,” Journal of Computer-Aided Molecular Design 13, 435-451, which is hereby incorporated by reference.

In some embodiments, algorithms such as DOCK (Shoichet, Bodian, and Kuntz, 1992, “Molecular docking using shape descriptors,” Journal of Computational Chemistry 13(3), pp. 380-397; and Knegtel et al., 1997 “Molecular docking to ensembles of protein structures,” Journal of Molecular Biology 266, pp. 424-440, each of which is hereby incorporated by reference) are used to find the one or more poses for a compound against a target macromolecule. Such algorithms model the macromolecule and the compound as rigid bodies. The docked conformation is searched using surface complementary to find poses.

In some embodiments, algorithms such as AutoDOCK (Morris et al., 2009, “AutoDock4 and AutoDockTools4: Automated Docking with Selective Receptor Flexibility,” J. Comput. Chem. 30(16), pp. 2785-2791; Sotriffer et al., 2000, “Automated docking of ligands to antibodies: methods and applications,” Methods: A Companion to Methods in Enzymology 20, pp. 280-291; and “Morris et al., 1998, “Automated Docking Using a Lamarckian Genetic Algorithm and Empirical Binding Free Energy Function,” Journal of Computational Chemistry 19: pp. 1639-1662, each of which is hereby incorporated by reference); FlexX (Rarey et al., 1996, “A Fast Flexible Docking Method Using an Incremental Construction Algorithm,” Journal of Molecular Biology 261, pp. 470-489, which is hereby incorporated by reference); GOLD (Jones et al., 1997, “Development and Validation of a Genetic Algorithm for flexible Docking,” Journal Molecular Biology 267, pp. 727-748, which is hereby incorporated by reference) are used to find one or more poses.

In some embodiments, molecular dynamics is performed on a target macromolecule (or a portion thereof such as the active site of the macromolecule) and a compound to identify one or more poses for the compound. During the molecular dynamics run, the atoms of the macromolecule and compound are allowed to interact for a fixed period of time, giving a view of the dynamical evolution of the system. In some embodiments, the trajectory of atoms in the target macromolecule and the compound are determined by numerically solving Newton's equations of motion for a system of interacting particles, where forces between the particles and their potential energies are calculated using interatomic potentials or molecular mechanics force fields. See Alder and Wainwright, 1959, “Studies in Molecular Dynamics. I. General Method,” J. Chem. Phys. 31 (2): 459; and Bibcode, 1959, J. Ch. Ph. 31, 459A, doi:10.1063/1.1730376, each of which is hereby incorporated by reference. Thus, in this way, the molecular dynamics run produces a trajectory of the macromolecule and the compound (e.g., initial compound, derived compound, etc.) over time. This trajectory comprises the trajectory of the atoms in the target macromolecule and the compound. In some embodiments, a subset of the plurality of different poses is obtained by taking snapshots of this trajectory over a period of time. In some embodiments, poses are obtained from snapshots of several different trajectories, where each trajectory comprises a different molecular dynamics run of the target macromolecule interacting with the compound. In some embodiments, prior to a molecular dynamics run, the compound is first docked into an active site of the target macromolecule using a docking technique.

As used herein, the term “parameter” refers to any coefficient or, similarly, any value of an internal or external element (e.g., a weight and/or a hyperparameter) in a model, regressor, and/or classifier that affects (e.g., modify, tailor, and/or adjust) one or more inputs, outputs, and/or functions in the model, regressor and/or classifier. For example, in some embodiments, a parameter refers to any coefficient, weight, and/or hyperparameter that is used to control, modify, tailor, and/or adjust the behavior, learning and/or performance of a model, regressor, and/or classifier. In some instances, a parameter is used to increase or decrease the influence of an input (e.g., a feature) to a model, regressor, and/or classifier. As a nonlimiting example, in some instances, a parameter is used to increase or decrease the influence of a node (e.g., of a neural network), where the node includes one or more activation functions. Assignment of parameters to specific inputs, outputs, and/or functions is not limited to any one paradigm for a given model, regressor, and/or classifier but can be used in any suitable model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for a model, regressor, and/or classifier (e.g., by error minimization and/or backpropagation methods, as described elsewhere herein).

In some embodiments, a model, regressor, and/or classifier of the present disclosure comprises a plurality of parameters. In some embodiments the plurality of parameters is n parameters, where n is an integer and n≥2, n≥5, n≥10, n≥25, n≥40, n≥50, n≥75, n≥100, n≥125, n≥150, n≥200, n≥225, n≥250, n≥350, n≥500, n≥600, n≥750, n≥1,000, n≥2,000, n≥4,000, n≥5,000, n≥7,500, n≥10,000, n≥20,000, n≥40,000, n≥75,000, n≥100,000, n≥200,000, n≥500,000, n≥1×10⁶, n≥5×10⁶, or n≥1×10⁷. In some embodiments n is between 10,000 and 1×10⁷, between 100,000 and 5×10⁶, or between 500,000 and 1×10⁶.

As used herein, the term “instruction” refers to an order given to a computer processor by a computer program. On a digital computer, in some embodiments, each instruction is a sequence of 0s and 1s that describes a physical operation the computer is to perform. Such instructions can include data transfer instructions and data manipulation instructions. In some embodiments, each instruction is a type of instruction in an instruction set that is recognized by a particular processor type used to carry out the instructions. Examples of instruction sets include, but are not limited to, Reduced Instruction Set Computer (RISC), Complex Instruction Set Computer (CISC), Minimal instruction set computers (MISC), Very long instruction word (VLIW), Explicitly parallel instruction computing (EPIC), and One instruction set computer (OISC).

As used herein, the term “graph neural network” (GNN) refers to a model that is suitable for representation learning of graphs. A GNN follow a neighborhood aggregation scheme, where the representation vector of a node is computed by recursively aggregating and transforming representation vectors of its neighboring nodes. After k iterations of aggregation, a node is represented by its transformed feature vector, which captures the structural information within the node's k-hop neighborhood. The representation of an entire graph can then be obtained through pooling, for example, by summing the representation vectors of all nodes in the graph. Input to a GNN includes molecular graphs, labeled graphs where the vertices and edges represent the atoms and bonds of the molecule, respectively. Graph neural networks and molecular graphs are further described, for example, in Xu et al., “How powerful are graph neural networks?” ICLR 2019, arXiv:1810.00826v3, which is hereby incorporated herein by reference in its entirety.

GNN variants for both node and graph classification tasks are known in the art. For example, in some embodiments, the first model is a graph convolutional neural network. Nonlimiting examples of graph convolutional neural networks are disclosed in Behler Parrinello, 2007, “Generalized Neural-Network Representation of High Dimensional Potential-Energy Surfaces,” Physical Review Letters 98, 146401; Chmiela et al., 2017, “Machine learning of accurate energy-conserving molecular force fields,” Science Advances 3(5):e1603015; Schutt et al., 2017, “SchNet: A continuous-filter convolutional neural network for modeling quantum interactions,” Advances in Neural Information Processing Systems 30, pp. 992-1002; Feinberg et al., 2018, “PotentialNet for Molecular Property Prediction,” ACS Cent. Sci. 4, 11, 1520-1530; and Stafford et al., “AtomNet PoseRanker: Enriching Ligand Pose Quality for Dynamic Proteins in Virtual High Throughput Screens,” chemrxiv.org/engage/chemrxiv/article-details/614b905e39ef6a1c36268003, each of which is hereby incorporated by reference.

Example Systems for Identifying One or More Compounds that Exhibit a Threshold Activity with Respect to a Target Macromolecule.

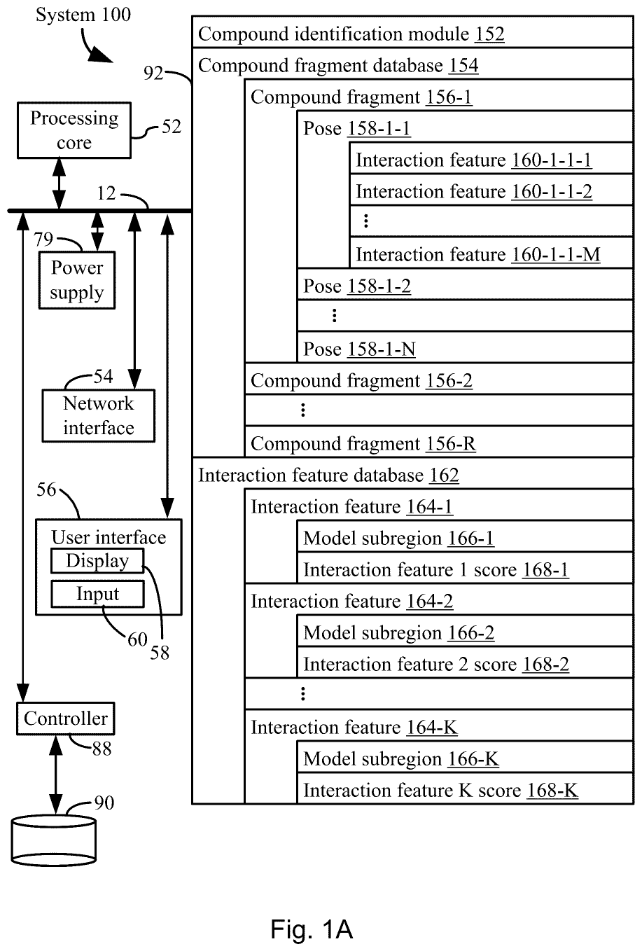

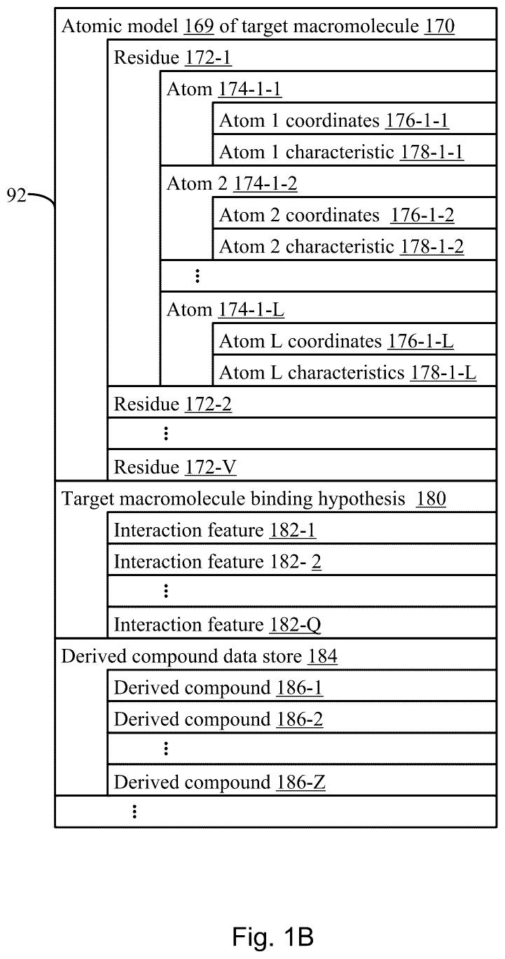

FIG. 1 illustrates a computer system 100 for identifying one or more compounds that exhibit a threshold activity with respect to a target macromolecule.

Referring to FIGS. 1A and 1B in typical embodiments, computer system 100 comprises one or more computers. For purposes of illustration in FIGS. 1A and 1, the computer system 100 is represented as a single computer that includes all of the functionality of the disclosed computer system 100. However, the present disclosure is not so limited. The functionality of the computer system 100 can be spread across any number of networked computers and/or reside on each of several networked computers and/or virtual machines. One of skill in the art will appreciate that a wide array of different computer topologies is possible for the computer system 100 and all such topologies are within the scope of the present disclosure.

Turning to FIGS. 1A and 1B with the foregoing in mind, the computer system 100 comprises one or more processing units (CPUs, processing cores) 52, a network or other communications interface 54, a user interface 56 (e.g., including an optional display 58 and optional keyboard 60 or other form of input device), a memory 92 (e.g., random access memory, persistent memory, or combination thereof), one or more magnetic disk storage and/or persistent devices 90 optionally accessed by one or more controllers 88, one or more communication busses 12 for interconnecting the aforementioned components, and a power supply 79 for powering the aforementioned components. To the extent that components of memory 92 are not persistent, data in memory 92 can be seamlessly shared with non-volatile memory 90 or portions of memory 92 that are non-volatile/persistent using known computing techniques such as caching. Memory 92 and/or memory 90 can include mass storage that is remotely located with respect to the central processing unit(s) 52. In other words, some data stored in memory 92 and/or memory 90 may in fact be hosted on computers that are external to computer system 100 but that can be electronically accessed by the computer system 100 over an Internet, intranet, or other form of network or electronic cable using network interface 54. In some embodiments, the computer system 100 makes use of models that are run from the memory associated with one or more graphical processing units in order to improve the speed and performance of the system. In some alternative embodiments, the computer system 100 makes use of models that are run from memory 92 rather than memory associated with a graphical processing unit.

In some embodiments, the memory 92 of the computer system 100 stores:
optional operating system (not shown in FIG. 1) that includes procedures for handling various basic system services;
compound identification module 152 for identifying one or more compounds that exhibit a threshold activity with respect to a target macromolecule;
a compound fragment database 154 storing compound fragments {156-1, 156-2, . . . , 156-R} where R is a positive integer, and for each such compound fragment 156 a corresponding plurality of poses (e.g., {158-1-1, 158-1-2, . . . , 158-1-N}, where N is a positive integer) of the respective compound fragment 156 against an atomic model of the target macromolecule, and for each such pose 158 a plurality of interaction features (e.g., {160-1-1-1, 160-1-1-2, . . . , 160-1-1-N}, where N is a positive integer) associated with the pose 158;
an interaction feature database 162 storing a plurality of interaction features {164-1, 164-2, . . . , 164-K} where K is a positive integer, and for each such interaction feature 164 a corresponding model subregion 166 associated with the interaction feature 164 and an interaction feature score 168;
an atomic model of a macromolecule 170 defined by a plurality of residues {172-1, 172-2, . . . , 172-V}, where V is a positive integer, and for each respective residue 172 in the plurality of residues, one or more atoms (e.g., {174-1-1, 174-1-2, . . . , 174-1-K}, where K is a positive integer) of the respective residue 172, and for each such atom 174, atom coordinates (e.g., coordinates {176-1-1, 176-1-2, . . . , 176-1-L}, where L is a positive integer) and characteristics (e.g., characteristics {178-1-1, 178-1-2, . . . , 178-1-L});
target macromolecule binding hypothesis 180 comprising a plurality of interaction features {182-1, 182-2, 182-Q} where Q is a positive integer; and
derived compound data store 184 comprising a plurality of derived compounds {186-1, 186-2, 186-A} where A is a positive integer.

In some implementations, any two or more of M, N, R, K, L, V, Q, or Z are the same or a different positive integer value. In some embodiments M, N, R, K, L, V, Q, or Z is a positive integer (e.g., 2, 3, 4, 5, 6, 7, 8, 9, 10, 11, 12, 13, 14, 15, 16, 17, 18, 19, or 20 or more). In some embodiments M, N, R, K, L, V, Q, or Z is a positive integer that is at least 1000, at least 5000, at least 10,000, at least 100,000, at least 1×10⁶, at least 1×10⁷, at least 1×10⁸, at least 1×10⁹, at least 1×10¹⁰, at least 1×10¹¹, or at least 5×10¹¹. In some embodiments, M, N, R, K, L, V, Q, or Z is a positive integer of no more than 1×10¹², no more than 1×10¹¹, no more than 1×10¹⁰, no more than 1×10⁹, no more than 1×10⁸, no more than 1×10⁷, no more than 1×10⁶, no more than 100,000, or no more than 10,000. In some embodiments, M, N, R, K, L, V, Q, or Z is a positive integer that is between 1000 and 100,000, 10,000 and 1×10⁷, 1×10⁶ and 1×10⁸, 1×10⁸ and 1×10¹¹, or 1×10⁹ and 1×10¹². In some embodiments, M, N, R, K, L, V, Q, or Z is a positive integer that falls within another range starting no lower than 10 and ending no higher than 1×10¹².

In some implementations, one or more of the above identified data elements or modules of the computer system 100 are stored in one or more of the previously mentioned memory devices, and correspond to a set of instructions for performing a function described above. The above identified data, modules, or programs (e.g., sets of instructions) need not be implemented as separate software programs, procedures or modules, and thus various subsets of these modules may be combined or otherwise re-arranged in various implementations. In some implementations, the memory 92 and/or 90 optionally stores a subset of the modules and data structures identified above. Furthermore, in some embodiments the memory 92 and/or 90 stores additional modules and data structures not described above.

Methods for Identifying One or More Compounds that Exhibit a Threshold Activity with Respect to a Target Macromolecule.

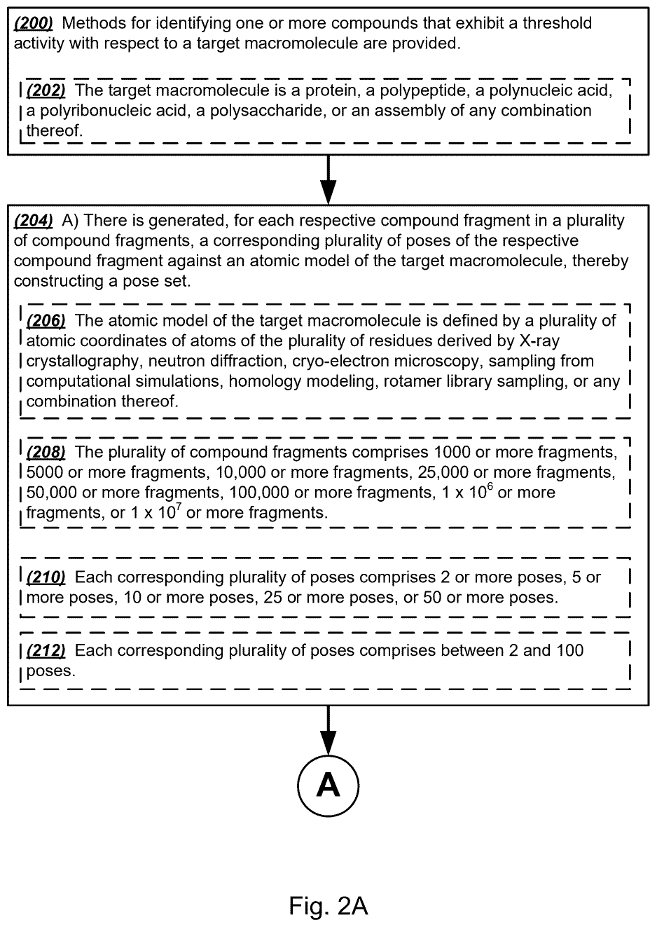

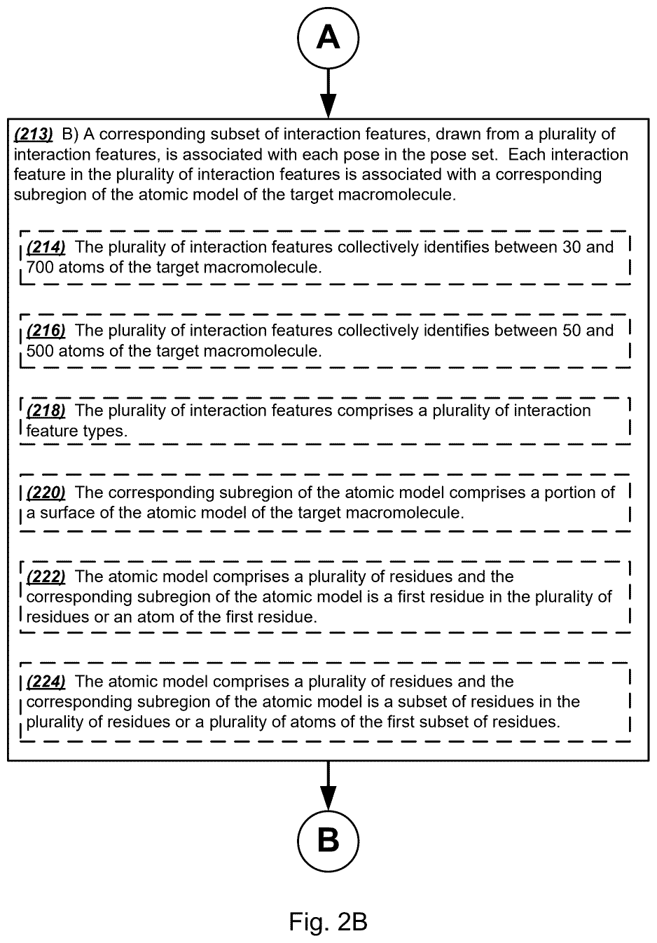

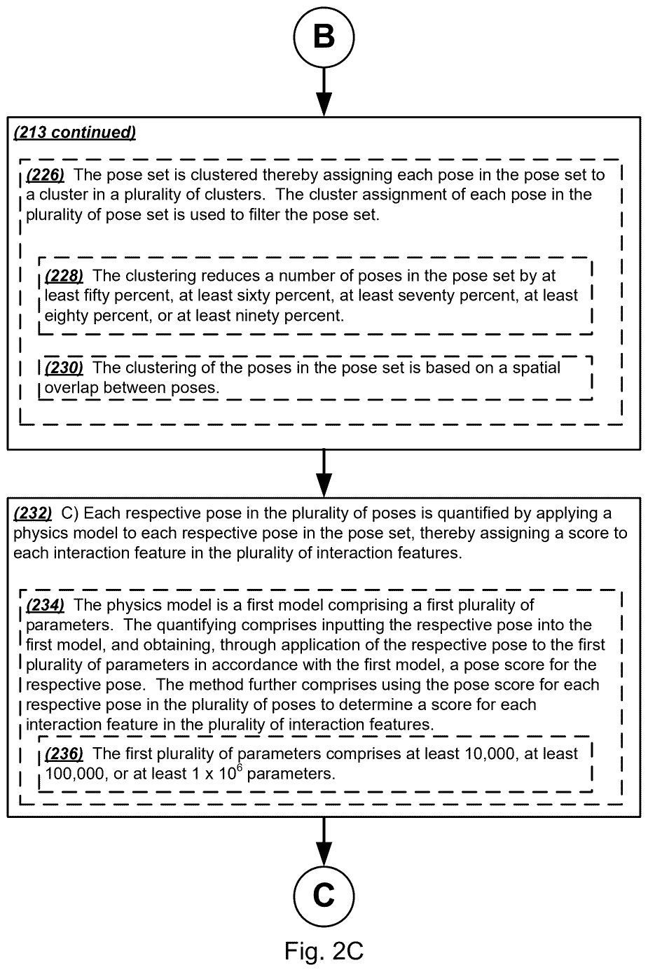

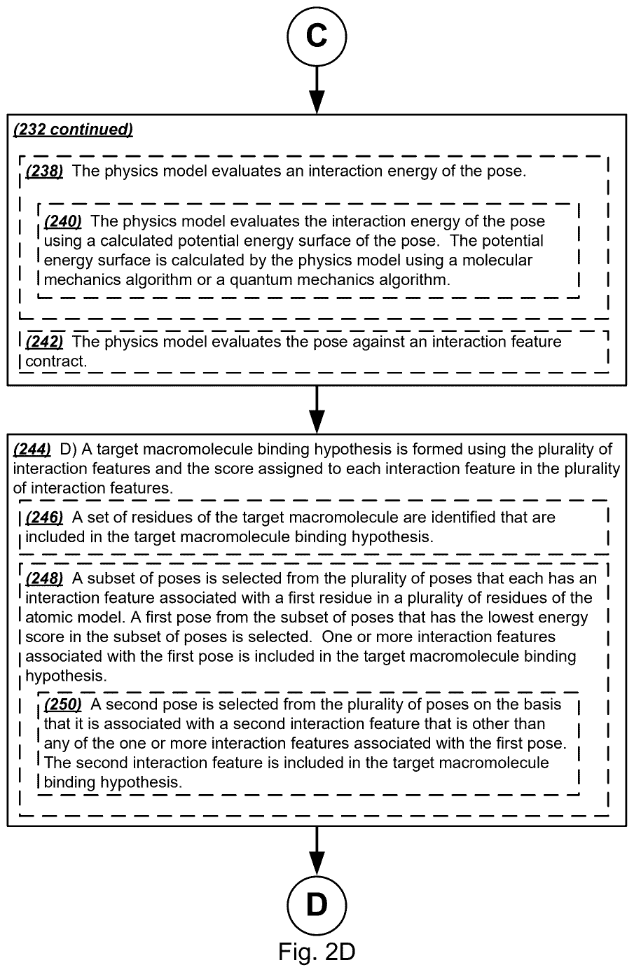

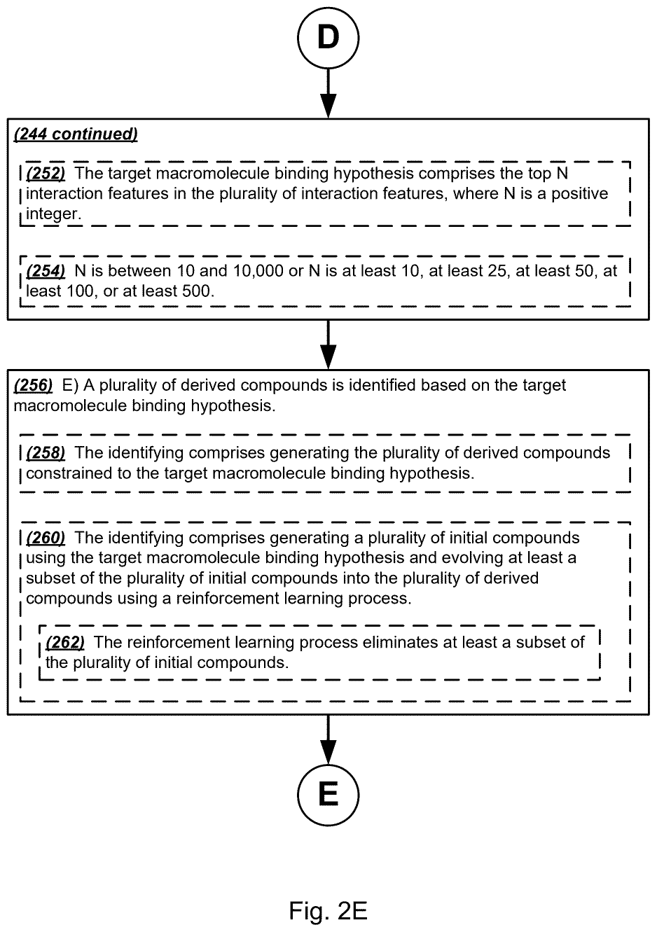

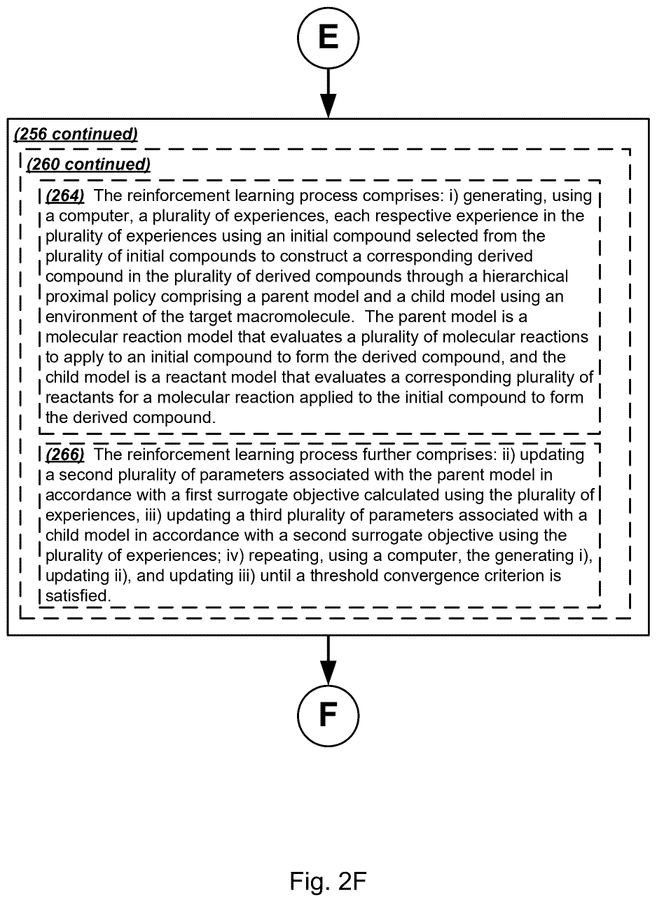

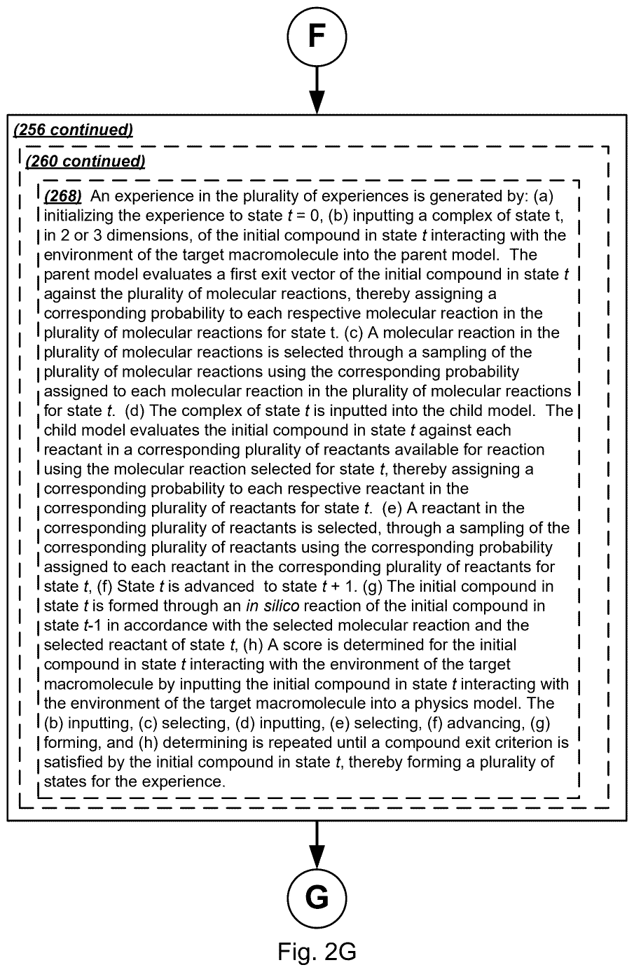

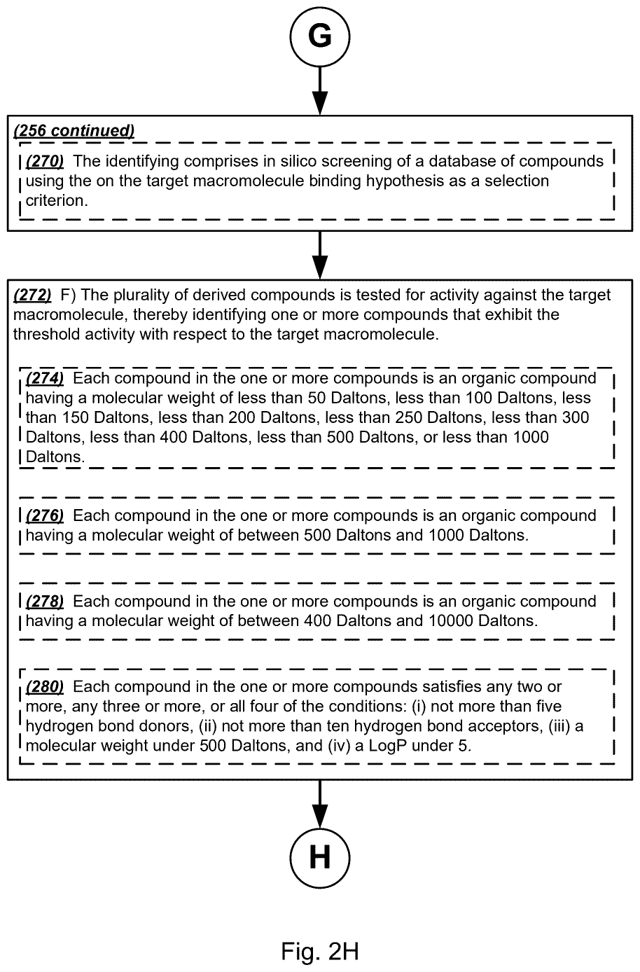

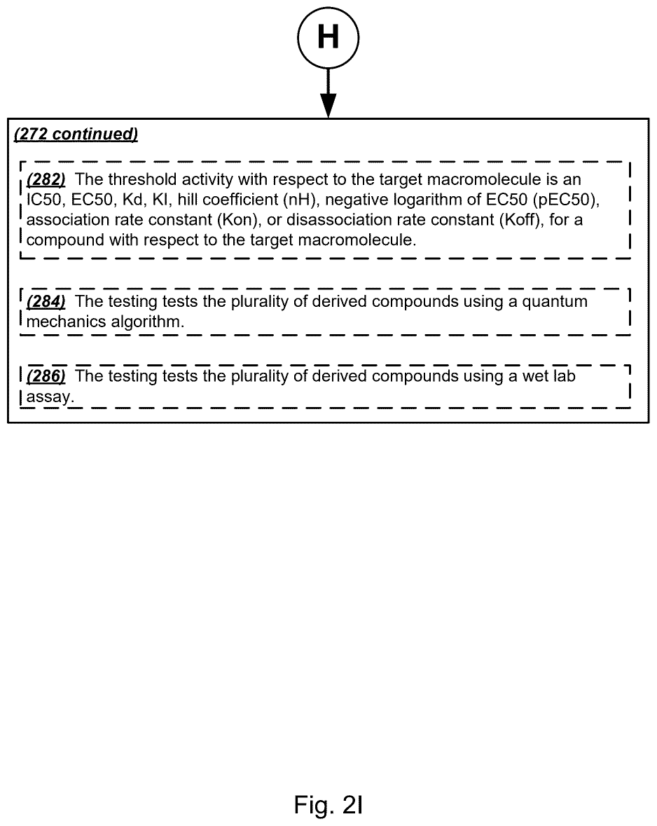

Now that a system for identifying one or more compounds that exhibit a threshold activity with respect to a target macromolecule has been described in conjunction with FIG. 1, an overview of a method for identifying one or more compounds that exhibit a threshold activity with respect to a target macromolecule is detailed with reference to FIG. 2.

Block 200. Referring to block 200 of FIG. 2A, methods for identifying one or more compounds that exhibit a threshold activity with respect to a target macromolecule 170 are provided. In some embodiments, as discussed above in conjunction with FIG. 1, the methods are performed at a computer system 100 comprising one or more processing cores 52 and a memory 92/90. In particular, in some embodiments of the present disclosure, the methods are performed by a compound identification module 152 resident on, or electronically accessible by, the computer system 100.

Block 202. Referring to block 202, in some embodiments, the target macromolecule 170 is a protein, a polypeptide, a polynucleic acid, a polyribonucleic acid, a polysaccharide, or an assembly of any combination thereof.

Block 602. Referring to block 602, in some embodiments, the target macromolecule 170 is a protein, a polypeptide, a polynucleic acid, a polyribonucleic acid, a polysaccharide, or an assembly of any combination thereof. In some embodiments, the target macromolecule 170 is a protein, a polypeptide, a polynucleic acid, a polyribonucleic acid, a polysaccharide, or an assembly of any combination thereof. In some embodiments, the target macromolecule 170 is a large molecule composed of repeating residues. In some embodiments, the target macromolecule 170 is a natural material. In some embodiments, the target macromolecule 170 is a synthetic material. In some embodiments, the target macromolecule 170 is an elastomer, shellac, amber, natural or synthetic rubber, cellulose, Bakelite, nylon, polystyrene, polyethylene, polypropylene, polyacrylonitrile, polyethylene glycol, or a polysaccharide.

In some embodiments, the target macromolecule 170 is a heteropolymer (copolymer). A copolymer is a polymer derived from two (or more) monomeric species, as opposed to a homopolymer where only one monomer is used. Copolymerization refers to methods used to chemically synthesize a copolymer. Examples of copolymers include, but are not limited to, ABS plastic, SBR, nitrile rubber, styrene-acrylonitrile, styrene-isoprene-styrene (SIS) and ethylene-vinyl acetate. Since a copolymer comprises at least two types of constituent units (also structural units, or particles), copolymers can be classified based on how these units are arranged along the chain. These include alternating copolymers with regular alternating A and B units. See, for example, Jenkins, 1996, “Glossary of Basic Terms in Polymer Science,” Pure Appl. Chem. 68 (12): 2287-2311, which is hereby incorporated herein by reference in its entirety. Additional examples of copolymers are periodic copolymers with A and B units arranged in a repeating sequence (e.g., (A-B-A-B-B-A-A-A-A-B-B-B)ₙ). Additional examples of copolymers are statistical copolymers in which the sequence of monomer residues in the copolymer follows a statistical rule. See, for example, Painter, 1997, Fundamentals of Polymer Science, CRC Press, 1997, p 14, which is hereby incorporated by reference herein in its entirety. Still other examples of copolymers that may be evaluated using the disclosed systems and methods are block copolymers comprising two or more homopolymer subunits linked by covalent bonds. The union of the homopolymer subunits may require an intermediate non-repeating subunit, known as a junction block. Block copolymers with two or three distinct blocks are called diblock copolymers and triblock copolymers, respectively.

In some embodiments, the target macromolecule 170 is a plurality of polymers (e.g., 2 or more, 3, or more, 10 or more, 100 or more, 1000 or more, or 5000 or more polymers), where the respective polymers in the plurality of polymers do not all have the same molecular weight. In some such embodiments, the polymers in the plurality of polymers share at least 50 percent, at least 60 percent, at least 70 percent, at least 80 percent, or at least 90 percent sequence identity and fall into a weight range with a corresponding distribution of chain lengths. In some embodiments, the target macromolecule 170 is a branched polymer molecule comprising a main chain with one or more substituent side chains or branches. Types of branched polymers include, but are not limited to, star polymers, comb polymers, brush polymers, dendronized polymers, ladders, and dendrimers. See, for example, Rubinstein et al., 2003, Polymer physics, Oxford; New York: Oxford University Press. p. 6, which is hereby incorporated by reference herein in its entirety.

In some embodiments, the target macromolecule 170 is a polypeptide. As used herein, the term “polypeptide” means two or more amino acids or residues linked by a peptide bond. The terms “polypeptide” and “protein” are used interchangeably herein and include oligopeptides and peptides. An “amino acid,” “residue” or “peptide” refers to any of the twenty standard structural units of proteins as known in the art, which include imino acids, such as proline and hydroxyproline. The designation of an amino acid isomer may include D, L, R and S. The definition of amino acid includes nonnatural amino acids. Thus, selenocysteine, pyrrolysine, lanthionine, 2-aminoisobutyric acid, gamma-aminobutyric acid, dehydroalanine, ornithine, citrulline and homocysteine, as nonlimiting examples, are all considered amino acids. Other variants or analogs of the amino acids are known in the art. Thus, a polypeptide may include synthetic peptidomimetic structures such as peptoids. See Simon et al., 1992, Proceedings of the National Academy of Sciences USA, 89, 9367, which is hereby incorporated by reference herein in its entirety. See also Chin et al., 2003, Science 301, 964; and Chin et al., 2003, Chemistry & Biology 10, 511, each of which is incorporated by reference herein in its entirety.

In some embodiments, the target macromolecule 170 includes any number of posttranslational modifications. Thus, in some embodiments, a target macromolecule 170 includes those polymers that are modified by acylation, alkylation, amidation, biotinylation, formylation, γ-carboxylation, glutamylation, glycosylation, glycylation, hydroxylation, iodination, isoprenylation, lipoylation, cofactor addition (for example, of a heme, flavin, metal, etc.), addition of nucleosides and their derivatives, oxidation, reduction, pegylation, phosphatidylinositol addition, phosphopantetheinylation, phosphorylation, pyroglutamate formation, racemization, addition of amino acids by tRNA (for example, arginylation), sulfation, selenoylation, ISGylation, SUMOylation, ubiquitination, chemical modifications (for example, citrullination and deamidation), and treatment with other enzymes (for example, proteases, phosphotases and kinases). Other types of posttranslational modifications are known in the art and are within the scope of the macromolecules or macromolecule complexes of the present disclosure.

In some embodiments, the target macromolecule 170 is a surfactant. Surfactants are compounds that lower the surface tension of a liquid, the interfacial tension between two liquids, or that between a liquid and a solid. Surfactants may act as detergents, wetting agents, emulsifiers, foaming agents, and dispersants. Surfactants are usually organic compounds that are amphiphilic, meaning they contain both hydrophobic groups (their tails) and hydrophilic groups (their heads). Therefore, a surfactant molecule contains both a water insoluble (or oil soluble) component and a water-soluble component. Surfactant molecules will diffuse in water and adsorb at interfaces between air and water or at the interface between oil and water, in the case where water is mixed with oil. The insoluble hydrophobic group may extend out of the bulk water phase, into the air or into the oil phase, while the water-soluble head group remains in the water phase. This alignment of surfactant molecules at the surface modifies the surface properties of water at the water/air or water/oil interface. Examples of ionic surfactants include ionic surfactants such as anionic, cationic, or zwitterionic (ampoteric) surfactants.

In some embodiments, the target macromolecule 170 is a reverse micelle or liposome. In some embodiments, the target macromolecule is a fullerene. A fullerene is any molecule composed entirely of carbon, in the form of a hollow sphere, ellipsoid or tube. Spherical fullerenes are also called buckyballs, and they resemble the balls used in association football. Cylindrical ones are called carbon nanotubes or buckytubes. Fullerenes are similar in structure to graphite, which is composed of stacked graphene sheets of linked hexagonal rings; but they may also contain pentagonal (or sometimes heptagonal) rings.

In some embodiments, the target macromolecule 170 includes two different types of polymers, such as a nucleic acid bound to a polypeptide. In some embodiments, the target macromolecule includes two polypeptides bound to each other. In some embodiments, the target macromolecule 170 includes one or more metal ions (e.g., a metalloproteinase with one or more zinc atoms).

In some embodiments, the target macromolecule 170 comprises 50 or more, 100 or more, 150 or more, 200 or more, 300 or more, 400 or more, 500 or more, 600 or more, 700 or more, 800 or more, 900 or more, 1000 or more, or 5000 or more atoms. In some embodiments, the target macromolecule 170 comprises no more than 10,000, no more than 5000, no more than 1000, no more than 500, or no more than 100 atoms. In some embodiments, the target macromolecule 170 consists of from 50 to 100, from 50 to 500, from 100 to 1000, or from 1000 to 10,000 atoms. In some embodiments, the target macromolecule 170 comprises another range of atoms starting no lower than 50 atoms and ending no higher than 10,000 atoms.

In some embodiments, the target macromolecule 170 is a polymer comprising 10 or more, 20 or more, 30 or more, 50 or more, 100 or more, or 500 or more residues. In some embodiments, the target macromolecule 170 is a polymer comprising no more than 1000, no more than 500, no more than 100, no more than 50, or no more than 20 residues. In some embodiments, the target macromolecule 170 is a polymer consisting of from 10 to 100, from 50 to 200, from 100 to 500, or from 500 to 1000 residues. In some embodiments, the target macromolecule 170 is a polymer that falls within another range starting no lower than 10 residues and ending no higher than 1000 residues.

In some embodiments, the target macromolecule 170 comprises one or more active sites to which a compound can bind.

### A) Generating a Corresponding Plurality of Poses of Each Respective Compound Fragment in a Plurality of Compounds Fragments Against an Atomic Model of the Target Macromolecule Thereby Constructing a Pose Set.

Block 204. Referring to block 204, for each respective compound fragment 156 in a plurality of compound fragments (e.g., compound fragment database 154), there is generated a corresponding plurality of poses 158 of the respective compound fragment against an atomic model 169 of the target macromolecule 170, thereby constructing a pose set.

In some embodiments, to perform block 204, the respective compound fragment 156 is first docked to the target macromolecule. A nonlimiting example of such docking programs is described above in conjunction with the definition of “pose” in the definitions section above. In some embodiments the respective compound fragment 156 is docked to a known active site of the target macromolecule. In some embodiments the active site of the compound fragment 156 is not known and the compound fragment 156 is docked to multiple sites on the atomic model of the target macromolecule. In some embodiments the target macromolecule has multiple active sites and the compound fragment 156 is docked to each such active site.

Block 206. Referring to block 206, in some embodiments, the atomic model of the target macromolecule is defined by a plurality of atomic coordinates of atoms of the plurality of residues derived by X-ray crystallography, neutron diffraction, cryo-electron microscopy, sampling from computational simulations, homology modeling, rotamer library sampling, or any combination thereof.

In some embodiments, the target macromolecule 170 is defined by a plurality of atomic coordinates {x₁, . . . , x_{N}} for a crystal structure of the target macromolecule 170 resolved at a resolution of 2.5 Å or better, where N is an integer of two or greater (e.g., 10 or greater, 20 or greater, etc.). In some embodiments, the target macromolecule 154 is a polymer and the spatial coordinates are a set of three-dimensional coordinates {x₁, . . . , x_{N}} for a crystal structure of the polymer resolved at a resolution of 3.3 Å or better (lower). In some embodiments, the target macromolecule 170 is defined by a plurality of atomic coordinates {x₁, . . . , x_{N}} for a crystal structure of the macromolecule resolved (e.g., by X-ray crystallographic techniques) at a resolution of 3.3 Å or lower, 3.2 Å or lower, 3.1 Å or lower, 3.0 Å or lower, 2.5 Å or lower, 2.2 Å or lower, 2.0 Å or lower, 1.9 Å or lower, 1.85 Å or lower, 1.80 Å or lower, 1.75 Å or lower, or 1.70 Å or lower.

In some embodiments, the spatial coordinates of the target macromolecule 170 are an ensemble of ten or more, twenty or more or thirty or more three-dimensional coordinates for the target macromolecule 170 determined by nuclear magnetic resonance where the ensemble has a backbone RMSD of 1.0 Å or lower, 0.9 Å or lower, 0.8 Å or lower, 0.7 Å or lower, 0.6 Å or lower, 0.5 Å or lower, 0.4 Å or lower, 0.3 Å or lower, or 0.2 Å or lower. In some embodiments the spatial coordinates of the target macromolecule 170 are determined by neutron diffraction or cryo-electron microscopy.

In some embodiments the spatial coordinates of the target macromolecule 170 are determined by a modeling program, such as AlphaFold2. AlphaFold2 is described in Jumper et al., 2021, “Highly accurate protein structure prediction with AlphaFold,” Nature 596, pp. 583-589; and Tunyasuvunakool et al., 2021, “Highly accurate protein structure prediction for the human proteome,” Nature 596, 590-596, each of which is hereby incorporated by reference.

Block 208. Referring to block 208, in some embodiments, the plurality of compound fragments comprises 1000 or more compound fragments, 5000 or more compound fragments, 10,000 or more compound fragments, 25,000 or more compound fragments, 50,000 or more compound fragments, 100,000 or more compound fragments, 1×10⁶ or more compound fragments, 1×10⁷ or more compound fragments, or 1×10⁸ or more compound fragments.

Advantageously, the systems and methods of the present disclosure are designed to evaluate a large number of compound fragments. In some embodiments, the plurality of compound fragments comprises at least 1000, at least 5000, at least 10,000, at least 100,000, at least 1×10⁶, at least 1×10⁷, at least 1×10⁸, at least 1×10⁹, at least 1×10¹⁰, at least 1×10¹¹, or at least 5×10¹¹ compound fragments. In some embodiments, the plurality of compound fragments comprises no more than 1×10¹², no more than 1×10¹¹, no more than 1×10¹⁰, no more than 1×10⁹, no more than 1×10⁸, no more than 1×10⁷, no more than 1×10⁶, no more than 100,000, or no more than 10,000 compound fragments. In some embodiments, the plurality of compound fragments consists of from 1000 to 100,000, from 10,000 to 1×10⁷, from 1×10⁶ to 1×10⁸, from 1×10⁸ to 1×10¹¹, or from 1×10⁹ to 1×10¹² compound fragments. In some embodiments, the plurality of compound fragments falls within another range starting no lower than 1000 compound fragments and ending no higher than 1×10¹² compound fragments.

Blocks 210-212. Referring to block 210, in some embodiments, each corresponding plurality of poses comprises 2 or more poses, 5 or more poses, 10 or more poses, 25 or more poses, or 50 or more poses. Block 212. Referring to block 212, in some embodiments, each corresponding plurality of poses comprises between 2 and 100 poses. In some embodiments, each corresponding plurality of poses comprises 2 or more poses, 10 or more poses, 100 or more poses, or 1000 or more poses of the respective compound fragment docked to the model 169 of the target macromolecule 170. Further discussion of such poses is described in the definitions section.

B) Associating a Corresponding Subset of Interaction Features, Drawn from a Plurality of Interaction Features, to Each Pose in the Pose Set.

Block 213. Referring to block 213, a corresponding subset of interaction features, drawn from a plurality of interaction features, is associated with each pose in the pose set, where each interaction feature in the plurality of interaction features is associated with a corresponding subregion of the atomic model of the target macromolecule. For example, the OEPerceiveInteractionHints function (OpenEye Scientific/Cadence Molecular Systems Interactions, Santa Fe, New Mexico) can be used to identify interaction hints in each of the poses with respect to the atomic model 169 of the target macromolecule, and each such interaction hint is an example of an interaction feature 160/164 in some embodiments. Additional example interaction features 160/182 that can be evaluated to determine whether they are present in a particular pose 158, in some embodiment of the present disclosure, are described in Bissantz et al., 2010, “A Medicinal Chemist's Guide to Molecular Interactions,” J. Med. Chem. 53, pp. 5061-5084, which is hereby incorporated by reference. These interaction features include, but are not limited to, hydrogen bonds, weak hydrogen bonds, halogen bonds, orthogonal multipolar interactions, hydrophobic interactions, aryl-aryl and alkyl-aryl interactions, cation −π interactions, and interactions formed by sulfur. Further examples of interaction features 160/164 are found in Tables 3-5 of Example 1, below. Further examples of interaction features are described in block 218 below.

Blocks 214-216. Referring to block 214, in some embodiments, the plurality of interaction features collectively identifies between 30 and 700 atoms of the target macromolecule. Referring to block 216, in some embodiments, the plurality of interaction features collectively identifies between 50 and 500 atoms of the target macromolecule.

This is because each interaction feature is associated with a corresponding subregion of the atomic model of the target macromolecule. For example, in Table 3 of Example 1, each of the interaction hints are associated with a particular residue of the atomic model of STAT6. In Table 4 of Example 1, each of the charge interactions are associated with a particular atom of a particular residue of the atomic model of STAT6. In Table 5 of Example 1, each of the hydrophobic interactions are associated with a particular atom of a particular residue of the atomic model of STAT6.

In some embodiments, an interaction feature is associated with a single atom of the atomic model of the target macromolecule. In some embodiments, an interaction feature is associated with a single residue of the atomic model of the target macromolecule. In some embodiments, an interaction feature is associated with all the atoms within a fixed distance of an atom of the atomic model of the target macromolecule. In some embodiments, this fixed distance is a number of angstroms in the range of between 0.5 Angstroms and 10 Angstroms, such as 0.5, 1.0, 1.5, 2, 2.5, 3, 4, 5, 6, 7, 8, 9, or 10 Angstroms.

Thus, in accordance with block 214, when all the regions associated with all the interaction features in the plurality of interaction features are considered, they collectively identify between 30 and 700 atoms of the atomic model of the target macromolecule. And, in accordance with block 216, when all the regions associated with all the interaction features in the plurality of interaction features are considered, they collectively identify between 50 and 500 atoms of the target macromolecule.

In alternative embodiments, when all the regions associated with all the interaction features in the plurality of interaction features are considered, they collectively identify between 10 and 2,200 atoms, between 30 and 1000 atoms, between 40 and 800 atoms, between 50 and 700 atoms, between 60 and 600 atoms, or between 70 and 500 atoms of the atomic model of the target macromolecule.

Block 218. Referring to block 218, in some embodiments, the plurality of interaction features comprises a plurality of interaction feature types. In some embodiments, interaction feature types include, but are not limited to, hydrophobic interactions, hydrophobic areas, aromatic ring members, hydrogen bond acceptors, hydrogen bond donors, hydrogen bond acceptor in an aromatic ring, negatively charged species, positively charged species, metal coordination, and/or halogen bonds. In some embodiments, an interaction feature type is a pharmacophore, such as a three-dimensional pharmacophore.

Three-dimensional pharmacophores have been used to capture the nature and three-dimensional arrangement of chemical functionalities in ligands that are relevant for molecular interactions with target macromolecules. Besides chemical nature and spatial arrangement, three-dimensional pharmacophores can capture feature directionality, such as in the case of hydrogen bonds and aromatic interactions. Additionally, spatial tolerance and weight can be fine-tuned for each pharmacophore feature to adjust its size and importance in the three-dimensional pharmacophore. In order to describe the preferable shape of molecules in an environment of the target macromolecule (e.g., binding site), pharmacophore features are often combined with exclusion volume constraints (also referred to as excluded volume constraints). For instance, an exclusion volume constraint can consist of a set of spheres that represent the protein residues imposing a barrier for binding of potential ligands.

Various tools are available in the art for modeling pharmacophores for ligand-target interactions (complex of the initial compound in state t interacting with the environment of the target macromolecule), including but not limited to FLAP, Pharmer, LigandScout, Catalyst, MOE, PHASE, Pharao, UNITY, and/or Forge. Three-dimensional pharmacophore elucidation methods can be classified as feature-based, substructure pattern-based, or molecular field-based, depending on how the pharmacophore features are derived. Feature-based methods derive pharmacophore features by filtering for geometric descriptors that match the characteristics of molecular interactions. Pattern-based methods, such as those implemented in PHASE, LigandScout, and Catalyst, detect substructures for chemical features in molecules. For example, all hydroxyl groups are defined as hydrogen bond donors and acceptors. In contrast, molecular field-based methods such as FLAP and Forge sample the molecular surface of either ligand or macromolecular target with different chemical probes and calculate interaction energy maps which can be translated into pharmacophore features. An additional distinction between three-dimensional pharmacophore generation methods is based on the type of employed data. This could be a set of active ligands, structural data on the ligand in complex with its macromolecular target, and/or structural data of the macromolecular target alone. Pharmacophores are further described, for example, in Schaller et al., “Next generation 3D pharmacophore modeling,” WIRES Comput Mol Sci. 2020; 10(4); Jiang and Rizzo, “Pharmacophore-based similarity scoring for dock,” J Phys Chem B. 2015; 119(3):1083-1102; and Arthur et al., “Hierarchical graph representation of pharmacophore models,” Front Mol Biosci. 2020; 7:599059, each of which is hereby incorporated herein by reference in its entirety.

In some embodiments, a respective interaction feature includes one or more corresponding geometric representations and/or one or more attribute values. In some embodiments, the dimensionality and nature of the geometric representations and/or attribute values of interaction features are dependent on the type of interaction feature; that is, a corresponding measurement appropriate for the respective interaction feature, as will be apparent to one skilled in the art. For instance, in some embodiments, a geometric representation of a respective interaction feature is a set of coordinates that indicates the position of the respective interaction feature in three-dimensional space for a respective conformation of the complex formed between an initial compound in state t and the environment of the target macromolecule. In some embodiments, a geometric representation of a respective interaction feature is a direction vector that indicates the direction or orientation of the respective interaction feature in three-dimensional space for the respective conformation of the complex formed between the of the initial compound in state t and the environment of the target macromolecule.

As another example, in some embodiments, an attribute value for a partial charge is a non-integer charge value when measured in elementary charge units; in yet another example, in some implementations, an attribute value for an aromatic ring pharmacophore includes a radius r of the aromatic ring.

Alternatively or additionally, in some embodiments, an attribute value for a respective interaction feature is a similarity score that measures a difference or a distance between the respective interaction feature in a complex formed between an initial compound in state t and the environment of the target macromolecule and a corresponding interaction feature in a reference conformation.

Alternatively or additionally, in some embodiments, an attribute value for a respective interaction feature is an indication of a presence or absence of the respective interaction feature at a corresponding position in a respective conformation of a complex formed between the initial compound in state t and the environment of the target macromolecule. In some embodiments, a corresponding geometric representation and/or a corresponding attribute value for a respective interaction feature is represented in a multi-dimensional space; for instance, in some embodiments, an attribute value for a hydrophobic interaction feature is represented as (1, 0, 0).

Interaction features are further described, for example, in Jiang and Rizzo, “Pharmacophore-based similarity scoring for dock,” J Phys Chem B. 2015; 119(3):1083-1102; and Arthur et al., “Hierarchical graph representation of pharmacophore models,” Front Mol Biosci. 2020; 7:599059, each of which is hereby incorporated herein by reference in its entirety.

Block 220. Referring to block 220, in some embodiments, the corresponding subregion of the atomic model comprises a portion of a surface of the atomic model of the target macromolecule.

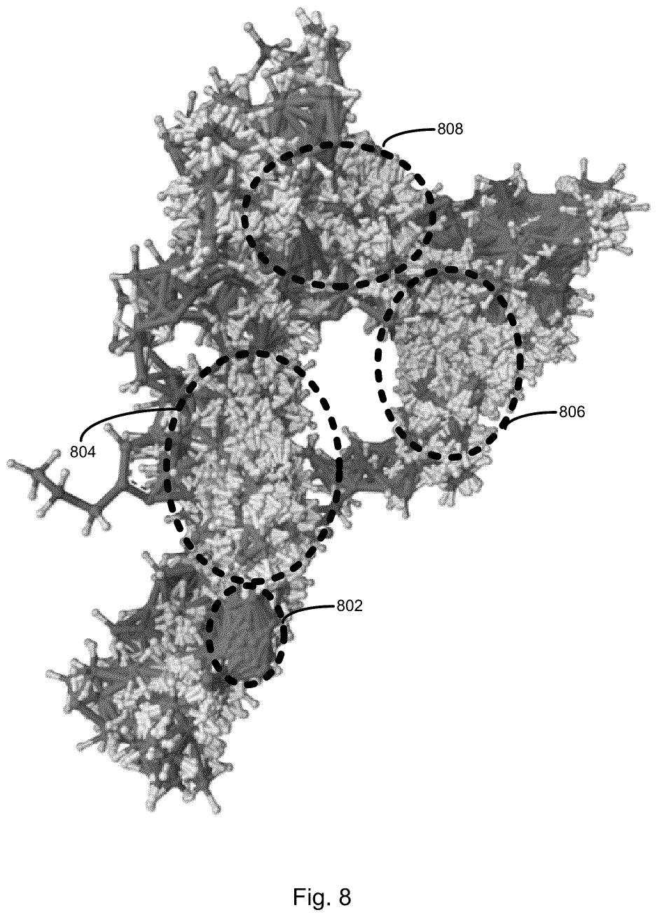

In some embodiments, the surface of the atomic model of the target macromolecule is defined as an accessible surface area (ASA), also known as the “accessible surface.” This is the surface area of an atomic model that is accessible to a solvent. Measurement of ASA is usually described in units of square Angstroms. ASA is described in Lee & Richards, 1971, J. Mol. Biol. 55(3), 379-400, which is hereby incorporated by reference herein in its entirety. ASA can be calculated, for example, using the “rolling ball” algorithm developed by Shrake & Rupley, 1973, J. Mol. Biol. 79(2): 351-371, which is hereby incorporated by reference herein in its entirety. This algorithm uses a sphere (of solvent) of a particular radius to “probe” the surface of the molecular system. The ASA associated with regions 802, 804, 806, and 808 of FIG. 8 are examples of portions of a surface of the atomic model.

In some embodiments, the surface of the atomic model of the target macromolecule is defined as the solvent-excluded surface, also known as the molecular surface or Connolly surface. The Connolly surface can be viewed as a cavity in bulk solvent (effectively the inverse of the solvent-accessible surface). It can be calculated in practice via a rolling-ball algorithm developed by Richards, 1977, Annu Rev Biophys Bioeng 6, 151-176 and implemented three-dimensionally by Connolly, 1992, J. Mol. Graphics 11(2), 139-141, each of which is hereby incorporated by reference herein in its entirety. The Connolly surface associated with regions 802, 804, 806, and 808 of FIG. 8 are examples of portions of a surface of the atomic model.

In some embodiments, the corresponding subregion of the atomic model associated with an interaction feature in the plurality of interaction features comprises a portion of a surface of the atomic model of the target macromolecule. In some embodiments, the corresponding subregion of the atomic model associated with an interaction feature in the plurality of interaction features comprises a portion of a surface of the atomic model of the target macromolecule that is between 10 Å² and 100 Å², between 5 Å² and 35 Å², between 3 Å² and 200 Å², between 1 Å² and 1000 Å², or between 0.2 Å² and 500 Å².

Block 222. Referring to block 222, in some embodiments, the atomic model comprises a plurality of residues and the corresponding subregion of the atomic model (associated with a particular interaction feature in the plurality of interaction features) is a first residue in the plurality of residues or an atom of the first residue. Non-limiting examples of interaction features that are each associated with a subregion of the atomic model that is a particular residue of the atomic model are found in Table 3 of Example 1.

Block 224. Referring to block 224, in some embodiments, the atomic model comprises a plurality of residues and the corresponding subregion of the atomic model is a subset of residues in the plurality of residues or a plurality of atoms of the first subset of residues. As an example, in some embodiments the corresponding subregion of the atomic model that is associated with a particular interaction feature in the plurality of interaction features is all the atoms of the atomic model that are within a threshold distance of a particular three-dimensional coordinate. This particular three-dimensional coordinate can, for example, be a coordinate of a particular atom in the atomic model. For instance, referring to the first row of Table 4, an example of a coordinate that is the location of a particular atom in the atomic model is the three-dimensional coordinates for STAT6 SER 565 (OG). Thus, in an exemplary embodiment, the subset of residues in the plurality of residues or a plurality of atoms of the first subset of residues of block 224 are all the atoms within a particular distance of STAT6 SER 565 (OG), such as within 3 Å, within 4 Å, within 5 Å, or within 6 Å. In some embodiments, the subset of residues in the plurality of residues or a plurality of atoms of the first subset of residues of block 224 are all the atoms within 3 Å, within 4 Å, within 5 Å, or within 10 Å of a particular coordinate. In some embodiments, the subset of residues in the plurality of residues or a plurality of atoms of the first subset of residues of block 224 are all the atoms within 3 Å, within 4 Å, within 5 Å, or within 10 Å of a particular atom in the atomic model of the target macromolecule. In some embodiments, the subset of residues in the plurality of residues or a plurality of atoms of the first subset of residues of block 224 are all the atoms within 3 Å, within 4 Å, within 5 Å, or within 10 Å of the coordinate of the center of mass of a particular residue in the atomic model of the target macromolecule.

Blocks 226-230. Referring to block 226, in some embodiments, the pose set is clustered thereby assigning each pose in the pose set to a cluster in a plurality of clusters. The cluster assignment of each pose in the pose set is used to filter the pose set. An example of such clustering is described in Example 1. There, clustering was based off of spatial overlap between poses in the pose set by defining a two Angstrom radius around each atom and counting poses that had fewer than a 50% overlap of these spheres as separate. This is and example of block 230, in which the clustering of the plurality of compound fragments is based on a spatial overlap between poses in the pose set. In some alternative embodiments, clustering is based off of spatial overlap between poses in the pose set by defining a 3, 4, 5, or 10 Angstrom radius around each atom and counting poses that had fewer than 60%, fewer than 50% or fewer than 40% overlap of these spheres as separate.

Referring to block 228, in some embodiments, the clustering reduces a number of poses in the pose set by at least fifty percent, at least sixty percent, at least seventy percent, at least eighty percent, or at least ninety percent. For instance, in generating FIG. 8 of Example 1, the lowest ranked pose for each of the clusters determined in FIG. 8 was used. Thus, in some embodiments, only the lowest ranked pose is retained in the pose set. Thus, in such embodiments, the clustering serves to filter the pose set to a unique set of poses that each represent the lowest overall interaction energy of their respective clusters. In some embodiments the metric used to rank poses within a cluster is the respective interaction energies between the compound fragments, in their particular poses, and the atomic model of the target macromolecule. In some embodiments the metric used to rank poses within a cluster is the respective physics model score of each of the particular poses, as determined by block 232 below.

### C) Quantifying Each Pose in the Plurality of Poses by Applying a Physics Model to Each the Pose, Thereby Score Each Interaction Feature in the Plurality of Interaction Features.

Block 232. Referring to block 232, each respective pose in the plurality of poses is quantified by applying a physics model to each respective pose in the pose set, thereby assigning a score to each interaction feature in the plurality of interaction features. In some embodiments the physical model considers, as input, the coordinates of the pose and at least the coordinates of the atomic model that are in the vicinity of the pose (e.g., within 3, 4, 5, or 10 Angstroms of an atom of the pose).

Block 234. Referring to block 234, in some embodiments, the physics model is a first model comprising a first plurality of parameters. The quantifying comprises inputting the respective pose into the first model, in addition to at least the coordinates of the atomic model that are in the vicinity of the pose (e.g., within 3, 4, 5, or 10 Angstroms of an atom of the pose), and obtaining, through application of the respective pose to the first plurality of parameters in accordance with the first model, a pose score for the respective pose. The method further comprises using the pose score for each respective pose in the plurality of poses to determine a score for each interaction feature in the plurality of interaction features. For instance, consider the case where interaction feature 1 is present in poses that have low (favorable) pose scores, as determined by the physics model, whereas interaction feature 2 is present in poses that have poor (unfavorable pose scores). In this instance, interaction feature 1 would be associated with favorable poses and would thus have a better score than interaction feature 2.

To score the interaction features based on their presence in scored poses, one of skill in the art will appreciate that several different approaches can be taken, each of which is within the scope of the present disclosure.

One such method is a weighted sum approach in which each pose 158 has a score. This pose score is distributed across the interaction features that are present in the pose. Thus, each interaction feature gets a score proportional to the scores of the poses it appears in. In particular, for each interaction feature 160, identify the set of poses where it appears. The sum of the scores of those poses is then used as the score of the interaction feature. Optionally, this interaction score is normalized by the number of poses in which the interaction feature appears, or by the total score of all poses. For example, if an interaction feature 160 is present in three poses 158, scored as 2, 4, and 6, the score of the interaction feature would be 2+4+6=12, or an average score of 12/3=4.

Another such method is a frequency-weighted approach in which each pose 158 has a score. Each interaction feature is scored based on how frequently it appears in poses and how highly those poses are scored. In particular, for each interaction feature 160, a count of how often it appears in poses (i.e., its frequency), is made. This frequency is multiplied by the score of each pose. This gives a cumulative score that weighs frequent interaction features more heavily. As an example, consider the case in which an interaction feature 160 appears in poses 158 scored as 2, 5, and 7, and appears twice in each. In this instance, the interaction feature score would be weighted by 2×(2+5+7)=28.

Another such method is a statistical correlation approach in which correlation methods are used to evaluate the strength of association between the interaction features and the scores of the poses they appear in. Each interaction feature's presence in poses is treated as a binary variable (1 if present, 0 if absent). The correlation coefficient (e.g., Pearson, Spearman) is then computed between the presence of the interaction feature 160 and the score of the corresponding pose. Features with high positive correlations are considered more strongly associated with high scores. As an example, if the correlation coefficient between an interaction feature 160 and the pose scores is 0.8, it means this feature is strongly associated with higher-scored poses.

Another method is a logistic regression/classification approach. That is, the task of determining interaction feature scores from the pose scores is treated as a classification task where the presence of high-scoring poses is modeled based on the interaction features. In such an approach, a model is created where the independent variables are the interaction features (binary indicators for presence/absence of interaction features) and the dependent variable is the score of the pose. Logistic regression or another classification method is then used to determine the probability that a pose with specific interaction features will have a high score. The coefficients from the model will give insight into how important each interaction feature is to the scoring. For instance, if the model (e.g., logistic regression) assigns a high coefficient to an interaction feature, it suggests this interaction feature contributes to a higher score.

Still another method is principal component analysis (PCA). In such an approach, PCA is used to reduce the dimensionality of the interaction features and poses to find the most significant interaction features that contribute to variation in the scores. In such an approach PCA is performed on the interaction features across poses, treating each pose as a data point. Interaction features that have the highest loadings on the principal components are identified as most associated with high-scoring poses. In other words, if a principal component that explains much of the variation in pose scores has high loadings for specific interaction features, these features are deemed important for high scores.

Still another method is mutual information gain, in which the mutual information between each interaction feature and the pose scores is calculated to determine how much information the presence of an interaction feature contributes to predicting the pose score. In such an approach, for each interaction feature, the mutual information is computed between the binary presence of the interaction feature in poses and their pose scores. The features are then ranked based on their mutual information scores. Features that have high mutual information scores contribute significantly to the variability in pose scores.

Still another method is to use a Bayesian Inference approach to estimate how likely it is that the presence of an interaction feature leads to a higher pose score. In such approaches, the probability of a high score given the presence of an interaction feature is modeled. Prior knowledge of feature distributions is used and updated beliefs based on the observed pose scores. If the posterior probability of high scores is significantly greater when a specific interaction feature is present, this feature is considered valuable.

These approaches offer different ways to weigh and rank interaction features based on their associations with scored poses. Additionally, visual inspection of interaction features present in poses can be done as illustrated in Example 1 to identify interaction features that should be included in a macromolecule binding hypothesis. Such visual inspection can be used to rank interaction features and such ranking can serve as scores for the interaction features.

Block 236. Referring to block 236, in some embodiments, the first plurality of parameters of the physics model comprises at least 10,000, at least 100,000, or at least 1×10⁶ parameters.

In some embodiments, the first plurality of parameters comprises at least 10, at least 100, at least 1000, at least 10,000, at least 100,000, at least 1×10⁶, at least 1×10⁷, or more parameters. In some embodiments, the first plurality of parameters consists of no more than 1×10⁸, no more than 1×10⁷, no more than 1×10⁶, no more than 100,000, no more than 10,000, no more than 1000, or no more than 100 parameters. In some embodiments, the first plurality of parameters consists of from 10 to 1000, from 100 to 100,000, from 10,000 to 1×10⁷, or from 1×10⁶ to 1×10⁸ parameters. In some embodiments, the first plurality of parameters falls within another range starting no lower than 10 parameters and ending no higher than 1×10⁸ parameters.

Block 238. Referring to block 238, in some embodiments, the physics model evaluates an interaction energy of the pose in order to provide a pose score. In some embodiments of block 238, the physics model evaluates an interaction energy of the pose using quantum mechanics, molecular mechanics with explicit solvent, molecular mechanics with a continuum solvent, or a heuristic model. Such quantum mechanics, molecular mechanics with explicit solvent, molecular mechanics with a continuum solvent, and heuristic models are summarized in Boas and Harbury, 2007, “Potential energy functions for protein design.” Current Opinion in Structural Biology. 17: 199-204, which is hereby incorporated by reference.

Block 240. Referring to block 240, in some embodiments, the physics model evaluates the interaction energy of the pose using a calculated potential energy surface of the pose. In some embodiments, the potential energy surface is calculated by the physics model using a molecular mechanics algorithm or a quantum mechanics algorithm.

In some such embodiments, the potential energy surface is calculated by the physics model using a molecular mechanics algorithm. Such molecular mechanics algorithms make use of molecular mechanics (MM) force fields, which are empirical models that describe the potential energy surfaces of molecular systems by treating them as collections of atomic point masses. These point masses interact via non-bonded and valence (bond, angle, and torsion) terms, which are typically parametrized to reproduce quantum chemical conformational energetics and physical properties. See, for example, Takaba et al., “Machine-learned molecular mechanics force fields from large-scale quantum chemical data,” arXiv:2307.07085v4 [physics.chem-ph]8 Dec. 2023; Davies et al., 2002, “Structure-based design of a potent purine-based cyclin-dependent kinase inhibitor, Nature structural biology 9(10), pp. 745-749; and Hagler, 2019, “Force field development phase ii: Relaxation of physics-based criteria . . . or inclusion of more rigorous physics into the representation of molecular energetics,” Journal of computer-aided molecular design, 33(2):205-264, each of which is hereby incorporated by reference. Example programs for implementing the physics model using a molecular mechanics algorithms include, but are not limited to GROMACS, AMBER, CHARMM, NAMD, Desmond, Large-scale Atomic/Molecular Massively Parallel Simulator (LAMMPS), and OpenMM. See, for example, Thompson et al., 2022, “LAMMPS—a flexible simulation tool for particle-based materials modeling at the atomic, meso, and continuum scales,” Comp Phys Comm 271 p. 10817, and Shirts, et al., 2017, “Lessons learned from comparing molecular dynamics engines on the SAMPL5 dataset,” J Comput Aided Mol Des. 31(1), pp. 147-161, each of which is hereby incorporated by reference.

In some such embodiments, the potential energy surface is calculated by the physics model using a quantum mechanics algorithm. Examples of quantum mechanics algorithm include, but are not limited to quantum mechanics-cluster (QM-Cluster), quantum mechanics/molecular mechanics (QM/MM), and continuum solvation methods. One review of such quantum mechanics algorithms is Ryde and Soderhjelm, 2016, “Ligand-Binding Affinity Estimates Supported by Quantum-Mechanical Methods,” Chem. Rev. 116, pp. 5520-5566, which is hereby incorporated by reference. Example programs for implementing the physics model using a quantum mechanics algorithm, include, but are not limited to Gaussian, ORCA, NWChem, GAMESS, Jaguar, and Psi4. See, for example, Peng et al., 2016, “Massively Parallel Implementation of Explicitly Correlated Coupled-Cluster Singles and Doubles Using TiledArray Framework,” The Journal of Physical Chemistry A 120(51), pp. 10231-10244, which is hereby incorporated by reference.

Block 242. Referring to block 242, in some embodiments, the physics model evaluates the pose against an interaction feature contract. As used herein, the term “interaction feature contract” comprise a listing of potential interaction features that can form between a compound fragment 156 docked to the atomic model 169 of the target macromolecule 170 in a particular pose. Nonlimiting examples of interaction features that can be found in the interaction feature contract include three-dimensional partial charges, three-dimensional pharmacophores, and/or molecular dynamics residue interaction time.

D) Forming a Target Macromolecule Binding Hypothesis Using the Plurality of Interaction Features and their Scores.

Blocks 244-246. Referring to block 244, a target macromolecule binding hypothesis is formed using the plurality of interaction features and the score assigned to each interaction feature in the plurality of interaction features. Block 246. Referring to block 246, in some embodiments, a set of residues of the target macromolecule are identified that are included in the target macromolecule binding hypothesis.

In some embodiments, a target macromolecule binding hypothesis is any subset of the plurality of interaction features. In some embodiments, a target macromolecule binding hypothesis is any subset of the plurality of interaction features, and the subregions of the atomic model of the target macromolecule associated with this subset of the plurality of interaction features. One example of a target macromolecule binding hypothesis is the first three rows of Table 3: (i) interaction feature “cationpi:ligandpi” associated with Lys544 of STAT6, (ii) interaction feature “salt-bridge:ligant-protein+” associated with Lys544 of STAT6, and (iii) interaction feature “hbond:protein2ligan” associated with SER566 of STAT6, where the interaction features are known OpenEye interaction hints. Another example of a target macromolecule binding hypothesis is the first three rows of Table 5: (i) a hydrophobe (interaction feature) 3.72 Å away from LYS 544 NZ, (ii) a hydrophobe 3.52 Å away from Pro 591 CD of STAT6 and (iii) a hydrophobe 3.89 Å away from Phe 592 CE1 of STAT6.

Block 248. Referring to block 248, in some embodiments, a subset of poses is selected from the plurality of poses that each has an interaction feature associated with a first residue in a plurality of residues of the atomic model. A first pose from the subset of poses that has the lowest energy score in the subset of poses is selected. For instance, using Example 1, all the poses that have an interaction features associated with Stat6 Lys 544 are considered. From these poses, the pose that has the lowest energy score (lowest first physics model score) is selected. One or more interaction features associated with this first pose is then included in the target macromolecule binding hypothesis.

Block 250. Referring to block 250, in some embodiments, a second pose is selected from the plurality of poses on the basis that it is associated with a second interaction feature that is other than any of the one or more interaction features associated with the first pose. The second interaction feature is included in the target macromolecule binding hypothesis. For instance, this interaction feature may be associated with a region that is distal to the first residue. By including this additional interaction feature, a basis for interacting with the model of the target macromolecule that is orthogonal (independent) of the interaction feature selected in block 248 is established, making the target macromolecule binding hypothesis more robust.

Blocks 252-254. Referring to block 252, in some embodiments, the target macromolecule binding hypothesis comprises the top N interaction features in the plurality of interaction features, where N is a positive integer. Block 254. Referring to block 254, in some embodiments, n is between 10 and 10,000 or N is at least 10, at least 25, at least 50, at least 100, or at least 500. Here “top” refers to those interaction features having the best interaction feature scores among the plurality of interaction features.

### E) Identifying a Plurality of Derived Compounds Based on the Target Macromolecule Binding Hypothesis.

Block 256. Referring to block 256, a plurality of derived compounds is identified based on the target macromolecule binding hypothesis.

Block 258. Referring to block 258, in some embodiments, the identifying comprises generating the plurality of derived compounds constrained to the target macromolecule binding hypothesis. For instance, in some embodiments this is done using reinforcement learning using the target macromolecule binding hypothesis as described in block 260. In alternative embodiments, this is done by in silico screening of a database of compounds using the target macromolecule binding hypothesis as described in block 270. In still other embodiments, this is done using a program such as Molgen, subject to the constraints imposed by the target macromolecule binding hypothesis.

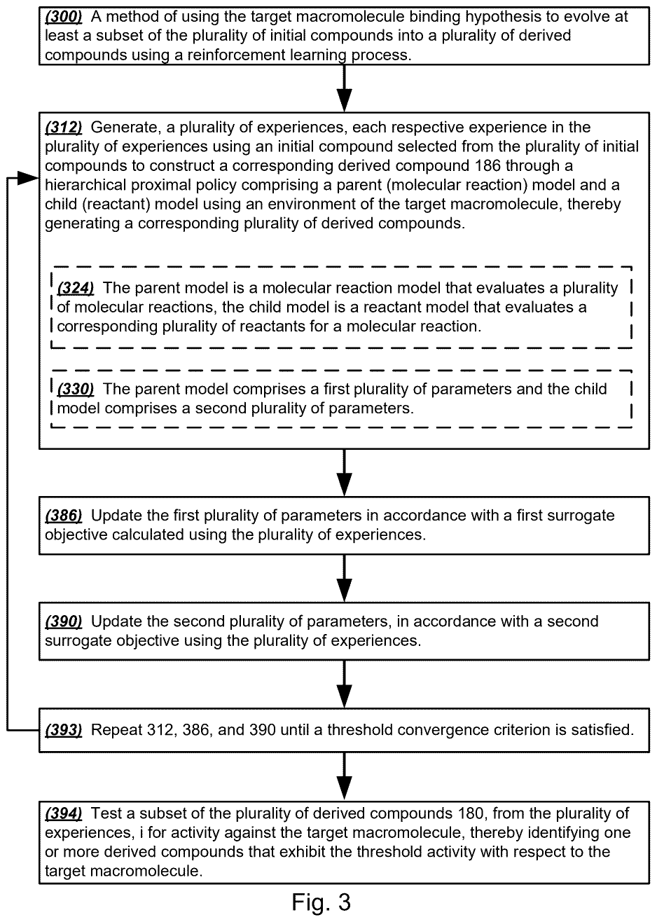

Blocks 260-262. Referring to block 260 of FIG. 2E and block 300 FIG. 3, in some embodiments, the identifying comprises generating a plurality of initial compounds using the target macromolecule binding hypothesis and evolving at least a subset of the plurality of initial compounds into the plurality of derived compounds using a reinforcement learning process. Reinforcement learning is further described, for example, in Sutton R S, Barto A G, “Reinforcement learning: an introduction,” IEEE Transactions on Neural Networks. 1998; 9(5):1054-1054, which is hereby incorporated herein by reference in its entirety. Example reinforcement learning for block 260 is described in U.S. Provisional Patent Application No. 63/696,258, entitled “Systems and Methods for Discovering Compounds Using Hierarchical Reinforcement Learning,” filed Sep. 18, 2024, which is hereby incorporated by reference. Referring to block 262 of FIG. 2E, in some embodiments, the reinforcement learning process eliminates at least a subset of the plurality of initial compounds.

Block 264 of FIG. 2F and in accordance with blocks 312 and 324 of FIG. 3, a plurality of experiences is generated. Each respective experience in the plurality of experiences uses an initial compound selected from the plurality of initial compounds to construct a corresponding derived compound through the hierarchical proximal policy comprising a parent (molecular reaction) model and a child (reactant) model using an environment of the target macromolecule, thereby generating a corresponding plurality of derived compounds.

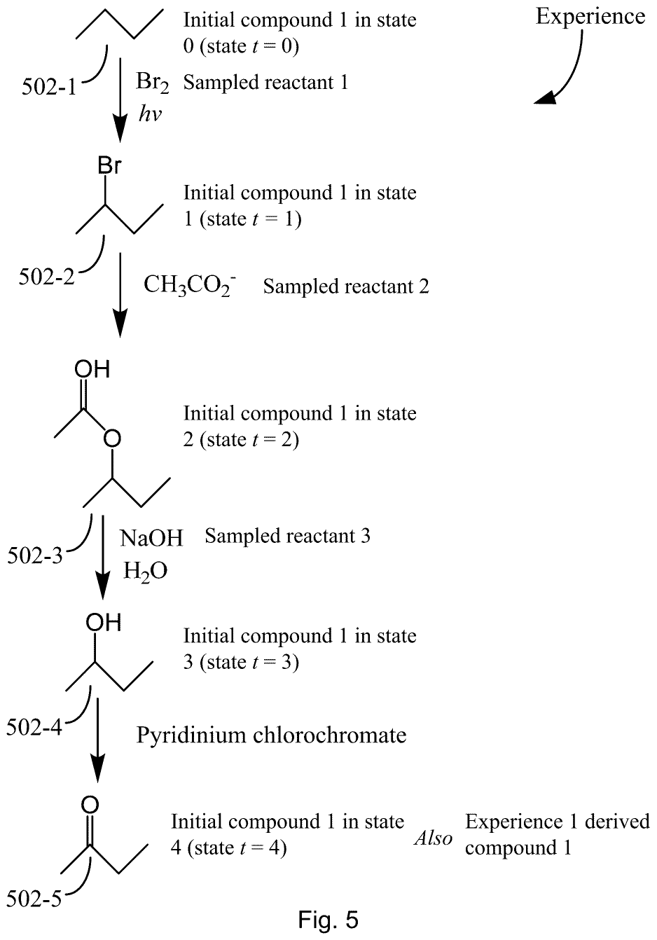

One such experience is illustrated in FIG. 5. Each respective experience in the plurality of experiences uses an initial compound in a plurality of initial compounds, constructed using the target macromolecule binding hypothesis, to derive a corresponding derived compound through the hierarchical proximal policy comprising the parent (molecular reaction) model and the child (reactant) model using the environment of the target macromolecule, thereby generating a corresponding plurality of derived compounds. In one nonlimiting example, a program such as Molgen is used to construct each initial compound in the plurality of initial compounds using the target macromolecule binding hypothesis. Molgen version 3.5, 4, or 5, Molgen-COMB, or MOLGEN-QSPR is used to perform this in silico reaction. See, for example, the Molgen Reference Guide, Version 5.0, Mar. 9, 2021, available on the Internet at https://molgen.de/documents/manual_molgen50.pdf, Gugisch et al., 2000, “MOLGENCOMB, a Software Package for Combinatorial Chemistry,” Commun. Math. Comput. Chem. 41 pp. 189-203; and Kerber et al., “MOLGEN-QSPR, a software package for the study of quantitative structure property relationships,” MATCH—Communications in Mathematical and in Computer Chemistry 51, each of which is hereby incorporated by reference. In some embodiments, alternatives to Molgen, such as RDKit, ChemAxon's Reactor, and Schrödinger's Maestro and Reaction-based Tools is used in block 654. See, for example Saldivar-Gonzilez et al., 2020, “Chemoinformatics-based enumeration of chemical libraries: a tutorial,” J Cheminform (2020) 12:64; and Landrum, 2020, “RDKit,” https://www.rdkit.org/, Accessed Aug. 29, 2024, each of which is hereby incorporated by reference.

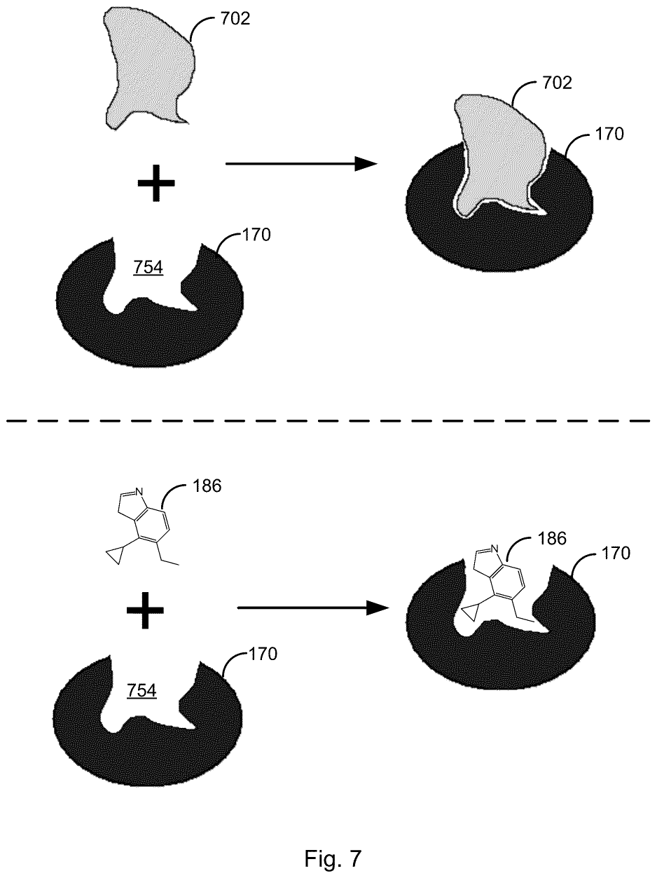

In some embodiments, the environment of the target macromolecule is a binding pocket of the target macromolecule 170. A stylized view of a target macromolecule 170 with an environment 754 that is a binding pocket is illustrated in FIG. 7, upper panel, in accordance with the prior art. Further illustrated in FIG. 7, upper panel is a natural ligand 702 for the target macromolecule 170, both before (FIG. 7, upper panel left), and after (FIG. 7, upper panel, right) forming a complex with the environment (binding pocket) of the target macromolecule 170. The goal of an experience is to derive a compound, such as compound 186 illustrated in to the lower panel of FIG. 7 that binds well to the environment of the target molecule.

In some embodiments, the environment of the target macromolecule 170 (e.g., a binding pocket) has a volume that ranges from 300 to 1,200 cubic angstroms (Å³). In some embodiments, the environment of the target macromolecule 170 has a volume that ranges from 250 to 5000 cubic Angstroms (Å³). In some embodiments, the environment of the target macromolecule 170 (e.g., a binding pocket) has a surface area that ranges between 400 and 1,200 square Angstroms (Å²).

In some embodiments, the environment of the target macromolecule 170 is defined by a plurality of atomic coordinates of atoms of residues of the binding pocket derived by X-ray crystallography, neutron diffraction, cryo-electron microscopy, sampling from computational simulations, homology modeling, rotamer library sampling, or any combination thereof.

In in some embodiments, the reinforcement learning process comprises: i) generating a plurality of experiences, each respective experience in the plurality of experiences using an initial compound selected from the plurality of initial compounds to construct a corresponding derived compound in the plurality of derived compounds through a hierarchical proximal policy comprising a parent model and a child model using the environment of the target macromolecule, where the parent model is a molecular reaction model that evaluates a plurality of molecular reactions to apply to an initial compound to form the derived compound, and the child model is a reactant model that evaluates a corresponding plurality of reactants for a molecular reaction applied to the initial compound to form the derived compound. With reference to FIG. 5, further details of such a reinforcement learning experience are described. The experience illustrated in FIG. 5 begins with an initial compound in state t=0 and culminates in a derived compound 186.

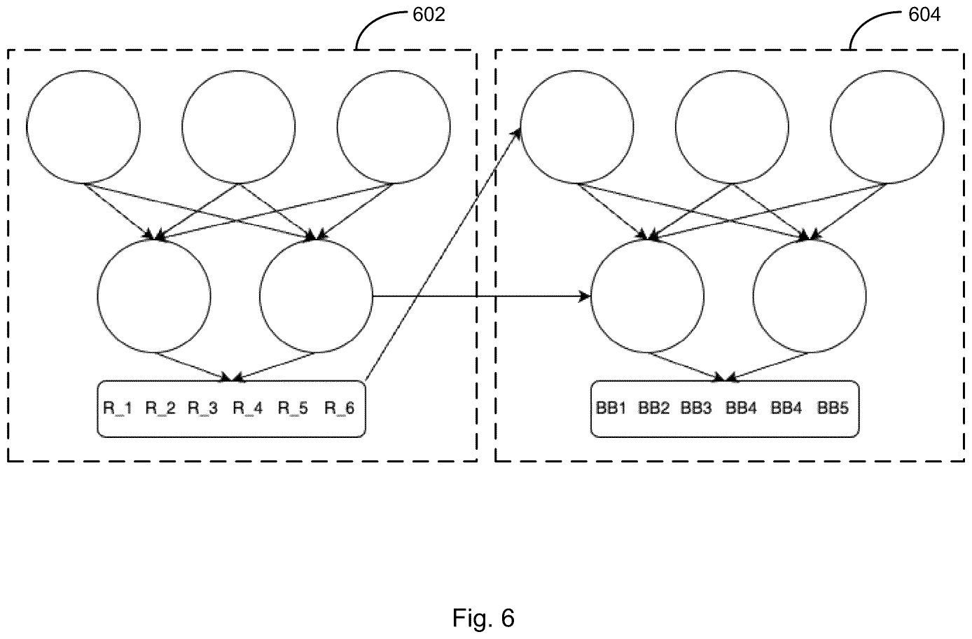

An example hierarchical relationship between an example parent model 602 and child model 604 is illustrated in FIG. 6. As illustrated in FIG. 6, the output of parent model 602 is a probability for each of six molecular reactions, R_1, . . . , R_6. The probabilities for R_1, . . . , R_6 sum to one. One of the molecular reactions R_1, . . . , R_6 is selected (sampled) on a probabilistic basis. For example, if the parent model assigned reaction R_1 a probability of 24%, there is a 24% chance that R_1 is selected. Next, the child model 604 takes the selected reaction and determines a probability for each reactant that could react with an initial compound in state t given the sampled molecular reaction. As illustrated in FIG. 6, the output of child model 604 is a probability for each of five reactants, BB_1, . . . , BB_5. The probabilities for BB_1, . . . , BB_5 sum to one. One of the reactants BB_1, . . . , BB_5 is selected (sampled) on a probabilistic basis. For example, if the child model assigned reactant BB_3 a probability of 14%, there is a 14% chance that BB_3 is selected.

In some embodiments, the parent model 602 is a first graph neural network (e.g., a first graph isomorphism neural network). Graph isomorphism networks are disclosed in Hu et al., 2018, “How Powerful are Graph Neural Networks,” cs>arXiv:1810.00826, which is hereby incorporated by reference.

In some embodiments, the parent model 602 is deep graph convolutional neural network (e.g., Zhang et al, “An End-to-End Deep Learning Architecture for Graph Classification,” The Thirty-Second AAAI Conference on Artificial Intelligence), GraphSage (e.g., Hamilton et al., 2017, “Inductive Representation Learning on Large Graphs,” arXiv:1706.02216 [cs.SI]), a graph isomorphism network (e.g., Hu et al., 2018, “How Powerful are Graph Neural Networks,” cs>arXiv:1810.00826, an edge-conditioned convolutional neural network (ECC) (e.g., Simonovsky and Komodakis, 2017, “Dynamic Edge-Conditioned Filters in Convolutional Neural Networks on Graphs,” arXiv:1704.02901 [cs.CV]), a differentiable graph encoder such as DiffPool (e.g., Ying et al., 2018, “Hierarchical Graph Representation Learning with Differentiable Pooling” arXiv:1806.08804 [cs.LG]), a message-passing graph neural network such as MPNN (Gilmer et al., 2017, “Neural Message Passing for Quantum Chemistry,” arXiv:1704.01212 [cs.LG]) or D-MPNN (Yang et al., 2019, “Analyzing Learned Molecular Representations for Property Prediction” J. Chem. Inf. Model. 59(8), pp. 3370-3388), or a graph neural network such as CMPNN (Song et al., “Communicative Representation Learning on Attributed Molecular Graphs,” Proceedings of the Twenty-Ninth International Joint Conference on Artificial Intelligence (IJCAI-20)). See also Rao et al., 2021, “MolRep: A Deep Representation Learning Library for Molecular Property Prediction,” doi.org/10.1101/2021.01.13.426489; posted Jan. 16, 2021. T; Rao et al., “Quantitative Evaluation of Explainable Graph Neural Networks for Molecular Property Prediction,” arXiv preprint arXiv:2107.04119; and github.com/biomed-AI/MolRep, for additional models that can be used as the parent model. In some embodiments, the parent model 602 has any of the architectures disclosed herein.

In some embodiments, the child model 604 is a second graph neural network (e.g., a second graph isomorphism neural network) that is passed an output of the parent model. In some embodiments, the architecture of the child model is the same or different than the architecture of the parent model and can have any of the architectures described for the parent model herein.

In accordance with block 330 of FIG. 3, the parent model 602 comprises a second plurality of parameters, and the child model 604 comprises a third plurality of parameters.

In some embodiments, the second plurality of parameters comprises at least 10, at least 100, at least 1000, at least 10,000, at least 100,000, at least 1×10⁶, at least 1×10⁷, or more parameters. In some embodiments, the second plurality of parameters consists of no more than 1×10⁸, no more than 1×10⁷, no more than 1×10⁶, no more than 100,000, no more than 10,000, no more than 1000, or no more than 100 parameters. In some embodiments, the second plurality of parameters consists of from 10 to 1000, from 100 to 100,000, from 10,000 to 1×10⁷, or from 1×10⁶ to 1×10⁸ parameters. In some embodiments, the second plurality of parameters falls within another range starting no lower than 10 parameters and ending no higher than 1×10⁸ parameters.

In some embodiments, the third plurality of parameters comprises at least 10, at least 100, at least 1000, at least 10,000, at least 100,000, at least 1×10⁶, at least 1×10⁷, or more parameters. In some embodiments, the third plurality of parameters consists of no more than 1×10⁸, no more than 1×10⁷, no more than 1×10⁶, no more than 100,000, no more than 10,000, no more than 1000, or no more than 100 parameters. In some embodiments, the third plurality of parameters consists of from 10 to 1000, from 100 to 100,000, from 10,000 to 1×10′, or from 1×10⁶ to 1×10⁸ parameters. In some embodiments, the third plurality of parameters falls within another range starting no lower than 10 parameters and ending no higher than 1×10⁸ parameters.

In some embodiments, the plurality of molecular reactions comprises named reactions, organic synthesis reactions or protecting group reactions.

In some embodiments, the plurality of molecular reactions comprises at least 10, at least 50, at least 100, at least 500, or at least 1000 molecular reactions. In some embodiments, the plurality of molecular reactions comprises no more than 5000, no more than 1000, no more than 100, no more than 50, or no more than 20 molecular reactions. In some embodiments, the plurality of molecular reactions consists of from 10 to 100, from 50 to 200, from 100 to 500, or from 500 to 5000 molecular reactions. In some embodiments, the plurality of molecular reactions falls within another range starting no lower than 10 molecular reactions and ending no higher than 5000 molecular reactions.

In some embodiments, the plurality of molecular reactions comprises one or more reaction SMILES (Simplified Molecular Input Line Entry Specification). SMILES representations comprise at least two fundamental types of symbols for atoms and bonds, respectively. These symbols are used to specify a molecular graph for a respective molecule (e.g., using “nodes” and “edges”) and assign labels to the components of the graph that indicate, for example, the type of atom each node represents and/or the type of bond each edge represents.

In some embodiments, a molecular reaction in the plurality of molecular reactions is represented by a Simplified Molecular Input Line Entry Specification (SMILES) arbitrary target specification ((SMARTS). SMARTS refers to a language that allows for the specification of molecular substructures using an extended set of rules. In particular, SMARTS uses atomic and bond symbols to specify a molecular graph, where the labels for the graph's nodes and edges (e.g., “atoms” and “bonds”) are extended to include “logical operators” and special atomic and bond symbols, thus allowing SMARTS atoms and bonds to be more general. Moreover, the SMARTS language can be used for the expression of molecular reactions (e.g., “reaction queries”). In some implementations, reaction queries are composed of optional reactant, agent, and product parts, which are separated by a “>” character. In such cases, the components of a reaction query match the corresponding roles within the reaction target. SMILES and SMARTS reactions are further disclosed, for example, in “SMARTS Theory Manual,” Daylight Chemical Information Systems, Santa Fe, New Mexico, available on the Internet at daylight.com/dayhtml/doc/theory/theory.smarts.html, which is hereby incorporated herein by reference in its entirety.

In some embodiments, the plurality of molecular reactions includes, but is not limited to, named reactions, organic synthesis reactions, protecting groups, total synthesis, Flow Chemistry, Green Chemistry, Microwave Synthesis, Multicomponent Reactions, Organocatalysis, and/or Sonochemistry. Alternatively or additionally, in some embodiments, the plurality of molecular reactions includes, but is not limited to, methyl esterification, hydrolysis of esters, amide synthesis, transamidation, oxidative amidation, Schmidt Reaction, Schotten-Baumann Reaction, Ugi Reaction, arylamine synthesis, Buchwald-Hartwig Reaction, Chan-Lam Coupling, Petasis Reaction, Ullmann Reaction, Hiyama Coupling, Kumada Coupling, Miyaura Borylation Reaction, Negishi Coupling, Stille Coupling, Suzuki Coupling, Sonogashira Coupling, Click Chemistry, Azide-Alkyne Cycloaddition, Copper-Catalyzed Azide-Alkyne Cycloaddition (CuAAC), Ruthenium-Catalyzed Azide-Alkyne Cycloaddition (RuAAC), Huisgen 1,3-Dipolar Cycloaddition, Synthesis of 1,2,3-Triazoles, epoxide synthesis, Jacobsen-Katsuki Epoxidation, Prilezhaev Reaction, Sharpless Epoxidation, Shi Epoxidation, and/or ring opening reactions of epoxides. Various molecular reactions are known in the art and are contemplated for use in the present disclosure. For instance, non-limiting examples of molecular reactions are further described in the Organic Chemistry Portal, available on the Internet at organic-chemistry.org.

In some embodiments, the corresponding plurality of reactants is a corresponding plurality of synthons. In some embodiments, the corresponding plurality of reactants comprises twenty or more reactants. Thus, in such embodiments, the child model evaluates and assigns a probability to each of twenty or more reactants, where the probabilities sum to one. For example, referring to state t=1 of FIG. 5 where a substitution reaction is selected, in instances where the corresponding plurality of reactants consists of twenty reactants, twenty different substitution groups (reactants) are evaluated for substituting out the bromide atom from the initial compound in state 1, and the child model assigns each of these substitution groups a probability, where the collective probabilities assigned to the twenty different substitution groups by the child model sum to one. The twenty different substitution groups are then sampled based on the assigned probabilities to select the actual substation that will be used in the chemical reaction selected in state 1 in order to build the initiation compound in state 2.

In some embodiments, the corresponding plurality of reactants comprises 20 or more synthons, 50 or more synthons, 100 or more synthons, 1000 or more synthons, 10,000 or more synthons, 100,000 or more synthons, or 1×10⁶ or more synthons. As used herein, a “synthon” refers to a representation of a chemical structure having an open valence (attachment bond) at, at least, one position. In some embodiments, synthons are derived from a reagent, from a synthetic reaction sequence, or from the fragmentation of a molecule (e.g., chemical structures derived from the disconnection of a bond). The potential universe of synthons can be vast. Synthons are building blocks or molecular fragments that can be combined in different ways to produce a wide range of compounds. In some embodiments the pool of possible synthons (e.g., in initial compound data store 158) considered represents more than 100, 500, 1000, 2000, 5000, 10,000, 11,000, 12,000, 13,000, 14,000, 15,000, or 20,000 synthons. In some embodiments these synthons might include various functional groups, heterocycles, and other structural motifs. In some embodiments, however, only those synthons, from this universe of synthons, that can work in the molecular reaction identified by the parent model, against a vector (reactive group) of the subject initial compound are considered by the child model during any given state of a particular experience.

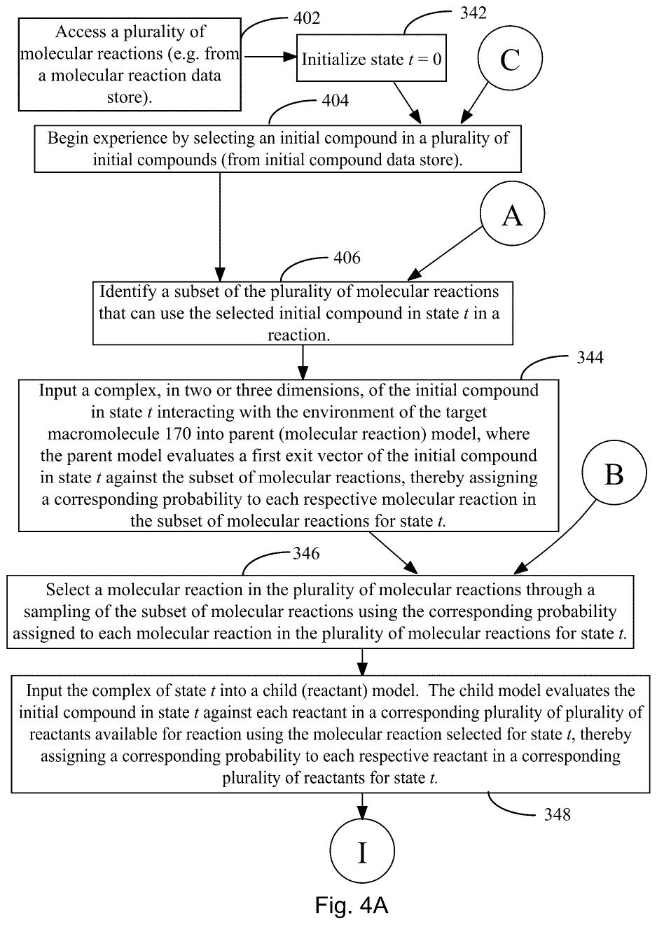

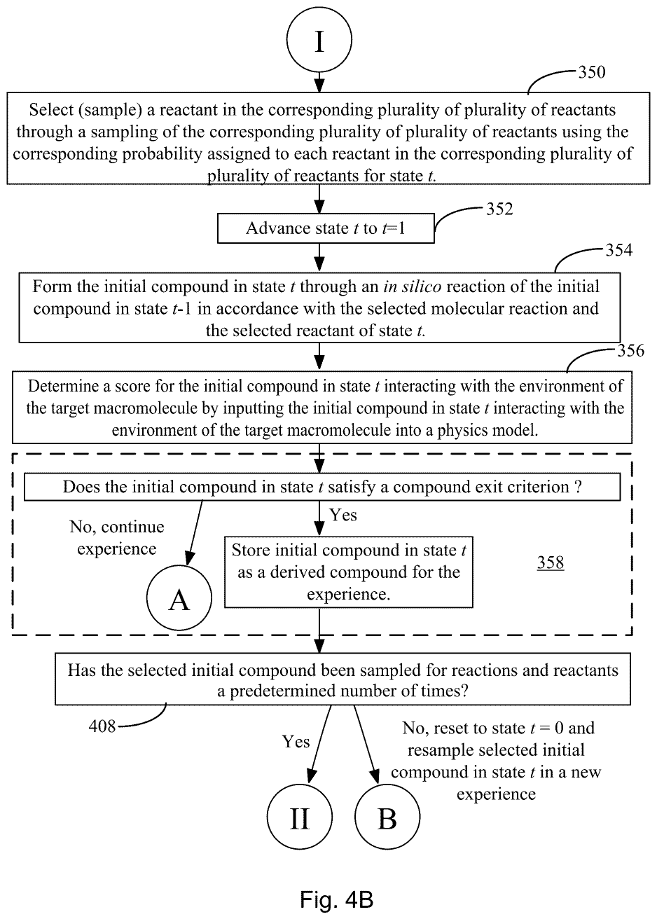

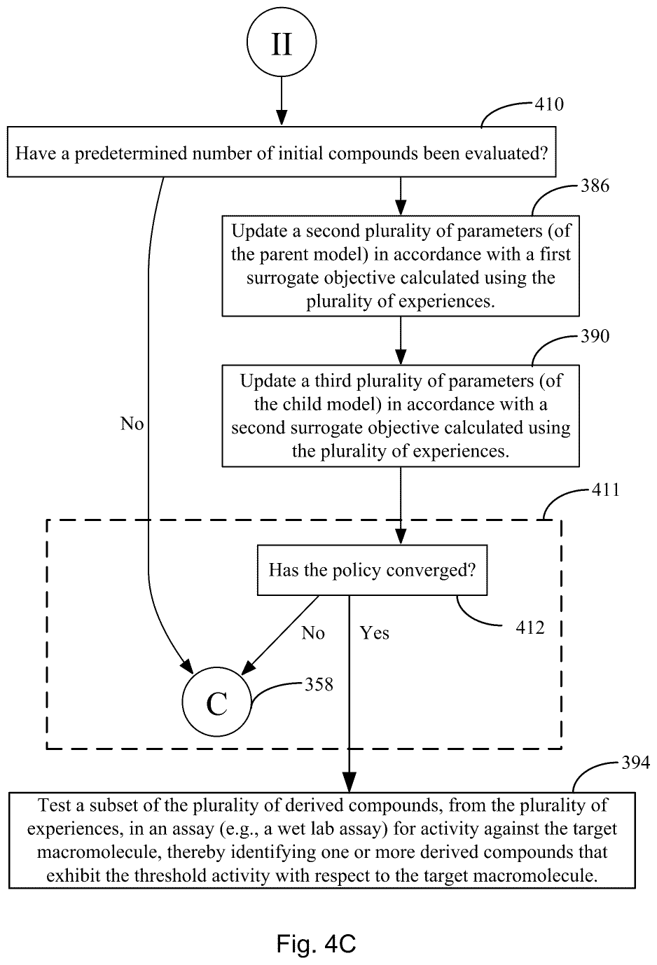

In some embodiments, the plurality of experiences is generated by the procedure outlined in FIGS. 4A, 4B, and 4C. At the outset, as illustrated in element 402 of FIG. 4A, a plurality of molecular reactions is accessed.

Block 342. At element 342 of FIG. 4A (i) the experience is initialized to state t=0, as illustrated in FIG. 4A. Referring to element 404 of FIG. 4A, state t=0 represents the selection of an initial compound before any in silico molecular reaction has been performed on the initial compound.

Referring to element 406 of FIG. 4A, in some embodiments, once an initial compound has been selected, the plurality of molecular reactions is filtered to identify a subset of molecular reactions that can make use of the selected molecular reaction. For example, referring to state t=0 in FIG. 5, one molecular reaction that can make use of the initial compound in state 0 is a halogenation reaction. Accordingly, a halogenation reaction is one of the molecular reactions that is included in the subset of molecular reactions in accordance with block 406 in some embodiments.

Referring to element 344 of FIG. 4A (ii) a complex, in two or three dimensions, of the initial compound in state t interacting with the environment of the target macromolecule 170 is inputted into the parent model.

In some embodiments, to perform element 344, the initial compound in state t is first docked into the environment (e.g., binding pocket) of the target macromolecule. A nonlimiting example of such docking programs is described above in conjunction with the definition of “pose” in the definitions section. The three dimensional coordinates of the complex of the compound in state t with the environment (e.g., binding pocket) of the target macromolecule is then inputted into a parent model in some embodiments. In alternative embodiments, the three dimensional coordinates of the complex of the compound in state t with the environment (e.g., binding pocket) of the target macromolecule is first converted into a two-dimensional graph and then inputted into the parent model. Example programs and techniques for generating a two-dimensional graph of a three dimensional complex are disclosed in Xu et al., “How powerful are graph neural networks?” ICLR 2019, arXiv:1810.00826v3, which is hereby incorporated herein by reference in its entirety. In such embodiments, the nodes of the graph typically represent atoms and the edges between the nodes represent bonds or interactions (e.g., covalent bonds, hydrogen bonds, or van der Waals interactions) between the atoms of the complex. In some such embodiments, the three-dimensional coordinates of the atoms of the initial compound complexed with the environment of the target macromolecule, and the information about their chemical environment (such as atom types, bond types, etc.) is fed into a model such as a graph neural network. The model encodes the spatial relationships and interactions from the three dimensional complex into a lower-dimensional representation. After processing the three-dimensional complex, the model can output a two-dimensional graph where the spatial information is implicitly captured in the node and edge features. This two-dimensional graph can, in turn, be evaluated by the parent model. The parent model evaluates a first exit vector of the initial compound in state t against the plurality of molecular reactions, thereby assigning a corresponding probability to each respective molecular reaction in the molecular reactions considered for state t. For instance, in FIG. 5, the bromine of the initial compound in state 1 is the exit vector considered in state 1 of the experience illustrated in FIG. 5. In some embodiments, the parent model evaluates and provides a probability for 2, 3, 4, 5, 6, 7, 8, 9, or 10 different molecular reactions. In some embodiments, the parent model evaluates and provides a probability for 10, 20, 30, 40, 50, 60, 70, 80, 90, or 100 or more different molecular reactions. In such embodiments these probabilities sum to one.

Referring to element 346 of FIG. 4A, (iii) a molecular reaction in the plurality of molecular reactions is selected, through a sampling of the plurality of molecular reactions using the corresponding probability assigned to each molecular reaction in the plurality of molecular reactions for state t. For instance, in the example illustrated in FIG. 6, the output of parent model 602 is a probability for each of six molecular reactions, R_1, . . . , R_6. The probabilities assigned by the parent model for R_1, . . . , R_6 sum to one. One of the molecular reactions R_1, . . . , R_6 is selected (sampled) on a probabilistic basis. For example, if the parent model 602 assigned reaction R_1 a probability of 24%, there is a 24% chance that R_1 is selected by element 346 of FIG. 4A.

Referring to element 348 of FIG. 3A (iv), the complex of state t is inputted into the child model 186.

In some embodiments, the complex of state t (the initial compound in state t docked into the environment of the target macromolecule) is in two or three dimensions in the same manner described for the input of the parent model in element 344 above.

The child model evaluates the initial compound in state t against each reactant in a corresponding plurality of reactants available for reaction using the molecular reaction selected for state t, thereby assigning a corresponding probability to each respective reactant in the corresponding plurality of reactants for state t. For example, as illustrated in FIG. 6, the child model 604 takes the selected molecular reaction of the parent model and the initial compound in state t (optionally complexed with the environment of the target macromolecule) and determines a probability for each reactant that could react with the initial compound in state t given this sampled molecular reaction. Referring to element 350 of FIG. 4B, (v) a reactant in the corresponding of plurality of reactants is selected through a sampling of the corresponding plurality of reactants using the corresponding probability assigned to each reactant in the corresponding plurality of reactants for state t. For instance, in the example illustrated in FIG. 6, the output of child model 604 is a probability for each of five reactants, BB_1, . . . , BB_5. The probabilities for BB_1, . . . , BB_5 sum to one. In accordance with block 350 of FIG. 4B, one of the reactants BB_1, . . . , BB_5 is selected (sampled) on a probabilistic basis. For example, if the child model 604 assigned reactant BB_3 a probability of 14%, there is a 14% chance that BB_3 is selected in element 350 of FIG. 4B. As discussed above, the actual number of reactants considered by the child model can be a number other than five.

In element 652 of FIG. 4B, (vi) the state is advanced from state t to state t+1 since a new molecule is about to be generated based on the initial compound at prior state t, the selected molecular reaction from element 346, and the selected reactant from element 350. In embodiments where the initial compound at prior state t has more than one vector (reactive atom or group), all other vectors are either removed from the initial compound at prior state t or are otherwise disregarded by the in silico synthesis.

In element 354 of FIG. 4B, (vii) the initial compound in state t is formed through an in silico reaction of the initial compound in state t−1 in accordance with the selected molecular reaction and the selected reactant of state t. In some embodiments, a program such as Molgen version 3.5, 4, or 5, Molgen-COMB, or MOLGEN-QSPR is used to perform this in silico reaction. See, for example, the Molgen Reference Guide, Version 5.0, Mar. 9, 2021, available on the Internet at https://molgen.de/documents/manual_molgen50.pdf, Gugisch et al., 2000, “MOLGENCOMB, a Software Package for Combinatorial Chemistry,” Commun. Math. Comput. Chem. 41 pp. 189-203; and Kerber et al., “MOLGEN-QSPR, a software package for the study of quantitative structure property relationships,” MATCH—Communications in Mathematical and in Computer Chemistry 51, each of which is hereby incorporated by reference. In some embodiments, alternatives to Molgen, such as RDKit, ChemAxon's Reactor, and Schrödinger's Maestro and Reaction-based Tools is used in block 654. See, for example Saldivar-Gonzilez et al., 2020, “Chemoinformatics-based enumeration of chemical libraries: a tutorial,” J Cheminform (2020) 12:64; and Landrum, 2020, “RDKit,” https://www.rdkit.org/, Accessed Aug. 29, 2024, each of which is hereby incorporated by reference.

In element 356 of FIG. 4B, (viii) a score for the initial compound in state t interacting with the environment of the target macromolecule 170 is determined by inputting the initial compound in state t interacting with the environment of the target macromolecule into a physics model.

In some embodiments, the score for the initial compound in state t interacting with the environment of the target macromolecule 170 characterizes or otherwise indicates an interaction between the initial compound and the environment of the target macromolecule 170.

In some implementations, the score is a causal interaction feature score that is obtained using one or more interaction features associated with a conformation of the initial compound 210 in state t when complexed to the target macromolecule 170.

In some implementations, the score is a causal interaction feature score that is obtained using the interaction features 182 within the target macromolecular binding hypothesis 180 that are associated with a conformation of the initial compound in state t when complexed to the target macromolecule 170.

In other embodiments, the score for the initial compound in state t interacting with the environment 154 of the target macromolecule 170 is an interaction score obtained by other methods, as will be apparent to one skilled in the art.

In some embodiments, the score for the initial compound in state t interacting with the environment of the target macromolecule 170 is based at least on a count of interaction features for a conformation of the initial compound in state t when complexed to the target macromolecule 170. A count of interaction features can refer to a tally of a plurality of interaction features associated with the initial compound in state t, but can also refer to any weighted count or computation of causality over the plurality of interaction features considered by the physics model. In some embodiments only those interaction features in the target macromolecule binding hypothesis are considered for such scoring. In some embodiments interaction features other than those in the target macromolecule binding hypothesis are considered for such scoring.

Accordingly, in some embodiments, the score for the initial compound in state t interacting with the environment of the target macromolecule is an absolute count, a weighted count, an individual treatment score (e.g., a dot product between an interaction feature vector and corresponding average treatment effects for each respective interaction feature in an interaction feature vector), a weighted individual treatment score, an efficiency score (e.g., a ratio of the number of interaction features for the respective molecule and the number of heavy atoms in the respective molecule), a weighted efficiency score, a diversity score (e.g., a measure of a diversity of interaction feature classes in a plurality of interaction features associated with the initial compound in state t interacting with the environment of the target macromolecule 170), and/or a weighted diversity score.

In some implementations, a weighted score gives greater import to one or more interaction features in a corresponding plurality of interaction features for the initial compound in state t, compared to other interaction features in the corresponding plurality of interaction features. In an example implementation, a weighted score gives greater weight to a first interaction feature that is selected as or known to be highly causal or associated with a particular property relevant to interaction (e.g., binding potency, selectivity, ADME properties, toxicity, etc.). In such an example implementation, the weighted score gives less weight to a second interaction feature that is selected as or known to be a covariate, confounder, or otherwise have lower causality for the particular property.

In some embodiments, the score is based, at least in part, on a calculated absorption, distribution, metabolism, and excretion (ADME) score. In some embodiments, an ADME model accepts, as input, a molecular fingerprint and/or a two-dimensional molecular graph of the initial compound in state t. Typically, drug development involves assessment of absorption, distribution, metabolism, and excretion (ADME) and/or toxicity (ADMET) to determine the effectiveness of an initial compound in state t as a drug. Such effectiveness is measured, in some implementations, as the ability of an initial compound in state t to reach its target in the subject in sufficient concentration, maintain bioactivity for long enough to achieve a target effect, and cause minimal toxicity. In some implementations, ADME or ADMET properties are determined using any one or more of a variety of techniques, including but not limited to substructure searches, molecular fingerprint methods, support vector machine (SVM) or Bayesian techniques, and/or deep neural networks. Various tools for predicting ADME or ADMET properties from the chemical structure of compounds are known in the art and provide indications of an initial compound in state t's physicochemical properties, pharmacokinetics, drug-likeness and/or medicinal chemistry friendliness, among others. Examples of such models include, but are not limited to, SwissADME, pk-CSN, admetSAR, iLOGP, BOILED-Egg, and/or Bioavailability Radar, each of which can be, or can contribute to the score of block 656.

Any number of ADME or ADMET models are contemplated for use in the present disclosure. For instance, available tools for predicting ADME or ADMET properties include those that focus on all or less than all ADME or ADMET properties. Accordingly, in some implementations, a plurality of ADME or ADMET models are used to determine a broad range of target properties, where each respective ADME or ADMET model outputs a corresponding measure of activity for the initial compound in state t that corresponds to one or more respective ADME or ADMET properties in a plurality of ADME or ADMET properties. ADME and ADMET models are further described, for example, in Daina et al., “SwissADME: a free web tool to evaluate pharmacokinetics, drug-likeness and medicinal chemistry friendliness of small molecules,” Sci Rep. 2017; 7(1):42717, which is hereby incorporated by reference in its entirety.

In some embodiments, the measure of activity determined to compute the score of element 356 includes a corresponding at least 1, at least 2, at least 3, at least 5, at least 10, or at least 20 measures of activity. In some embodiments, the corresponding measure of activity includes no more than 20, no more than 15, no more than 10, or no more than 5 measures of activity. In some embodiments, the corresponding measure of activity consists of from 1 to 5, from 2 to 10, from 5 to 18, or from 10 to 20 measures of activity. In some embodiments, the corresponding measure of activity falls within another range starting no lower than 1 and ending no higher than 20 measures of activity.

In some embodiments, a weighted score is differentially weighted based on the presence or absence of one or more interaction features in a corresponding plurality of interaction features for the initial compound in state t. In some embodiments each interaction feature in the corresponding plurality of interaction features is in the target macromolecular binding hypothesis 180. In some embodiments some of the interaction features in the corresponding plurality of interaction features is in the target macromolecular binding hypothesis 180. In some embodiments none of the interaction features in the corresponding plurality of interaction features is in the target macromolecular binding hypothesis 180.

In some such embodiments, a respective score for the initial compound in state t is predictive of binding when one or more interaction features, or classes thereof, in a first subset of interaction features is present in the corresponding plurality of interaction features for the initial compound in state t, and is not predictive of binding when none of the interaction features, or classes thereof, in the first subset of interaction features is present in the corresponding plurality of interaction features for the initial compound in state t. In other words, in some such embodiments, a weighted score accounts for interaction features or feature classes that are selected as or known to be essential for a particular interaction property. Alternatively or additionally, in some embodiments, a weighted score accounts for interaction features or feature classes that are selected as or known to be adverse or inhibitive to the particular interaction property. In some embodiments, a weighted score is determined by adjusting a corresponding attribute for each respective interaction feature by a weighting factor (e.g., 0.8, 0.2).

In some embodiments, a score for the initial compound in state t interacting with the environment of the target macromolecule 170 is obtained using a respective plurality of interaction features obtained for a complex formed between the initial compound in state t interacting with the environment 154 of the target macromolecule 170. In some embodiments each interaction feature in the respective plurality of interaction features is in the target macromolecular binding hypothesis 180. In some embodiments some of the interaction features in the respective plurality of interaction features is in the target macromolecular binding hypothesis 180. In some embodiments none of the interaction features in the respective plurality of interaction features is in the target macromolecular binding hypothesis 180.

One skilled in the art will appreciate that the interaction features used for calculating the score for the initial compound 210 in state t interacting with the environment 154 of the target macromolecule 170 can be obtained using any suitable method, including but not limited to a causal binding hypothesis generation method, a causal selectivity hypothesis generation method, a graph neural network for binding, and/or a graph neural network for selectivity. In some embodiments the interaction features used for calculating the score for the initial compound 210 in state t interacting with the environment 154 of the target macromolecule 170 are those in the target macromolecular binding hypothesis 180.

In some embodiments, the score for the initial compound in state t interacting with the environment of the target macromolecule is in fact a composite score formed from individual component scores. In some embodiments the score for the initial compound in state t interacting with the environment of the target macromolecule is determined by inputting the initial compound in state t interacting with the environment of the target macromolecule into each of a plurality of physics model, with each such physics model producing a component score that is aggregated to form the score for the initial compound in state t interacting with the environment of the target macromolecule. In some embodiments, there are 3, 4, 5, 6, 7, 8, 9, 10, 11, 12, 13, 14, or 15 or more physis models that each contribute a component score that is aggregated to form the score for the initial compound in state t interacting with the environment of the target macromolecule upon input of the initial compound in state t interacting with the environment of the target macromolecule.

In some embodiments, the score for the initial compound in state t interacting with the environment of the target macromolecule takes input (e.g., component score) from both the one or more physics models as well as other kinds of models.

For instance, in a first example, in some embodiments the two-dimensional structure of the initial compound in state t is used to ensure that the compound is within the ideal cheminformatics ranges such as a user specified log p range, a user specified molecular weight range, is user specified range of hydrogen acceptors, a user specified quantitative estimate of drug-likeness (QED) score, a scaffold diversity measure, etc. In some embodiments, one or more component scores from such cheminformatic checks contributes to the score of element 356.

In some embodiments reactive handles (vectors) on the initial compound in state t are replaced with carbons to ensure that that reactive handles are being classified as making interactions with the environment of the target macromolecule. The initial compound in state t is then docked to the environment of the target macromolecule. In some such embodiments a docking score for this docking contributes to the score of element 356.

In some embodiments, the docking identifies multiple poses of the initial compound in state t docked to the environment of the target macromolecule, each of which is scored, and each of which contributes to the score of element 356. In some embodiments, the best 5, 6, 7, 8, 9, 10, 15, 20, 25, 30, or 50 poses are taken and each contributes to the score of element 356.

In some embodiments, the single best pose or the top N poses, where N is a positive integer between 2 and 100, of the initial compound in state t docked to the environment of the target macromolecule are evaluated for interaction hits. In some embodiments, the interactions that are evaluated are specified in a causal interaction feature contract for the environment of the target macromolecule. Methods for identifying causal interaction features that can populate a causal interaction feature contract are disclosed in International Patent Application No. PCT/US24/24456, entitled “Systems and Methods for Discovering Compounds Using Causal Inference,” filed Apr. 12, 2024, which is hereby incorporated by reference. In some embodiments, one or more score for such interactions (e.g., one for each pose, or a composite of the poses) contributes to the score of element 356.

In some embodiments, the interaction energies between the single best pose or the top N poses and the environment of the target macromolecule are evaluated using quantum mechanical calculations. One example suitable program for this is disclosed in Gao et al., “TorchANI: A Free and Open Source PyTorch-Based Deep Learning Implementation of the ANI Neural Network Potentials,” ChemRxiv. 2020; doi:10.26434/chemrxiv.12218294.v1, which is hereby incorporated by reference. In some embodiments, one or more score for such interactions (e.g., one for each pose, or a composite of the poses) contributes to the score of element 356.

In some embodiments, non-covalent interactions between the single best pose or the top N poses of the initial compound in state t docked to the environment of the target macromolecule are evaluated using a symmetry-adapted perturbation theory (SAPT) zeroth-order approximation framework, which considers, for example, electrostatic interactions, exchange-repulsion interactions, induction, and dispersion of such complexes. One example suitable program for this is disclosed in Patkowski, 2019 “Recent developments in symmetry-adapted perturbation theory,” WIREs Computational Molecular Science 10(3), which is hereby incorporated by reference. In some embodiments, one or more score from such calculations (e.g., one for each pose, or a composite of the poses) contributes to the score of element 356.

In some embodiments, any combination of such scores is accumulated (aggregated) and used as the overall score computed in element 356. In some embodiments, the overall score is a measure of central tendency (e.g., mean, median, mode, weighted mean, weighted median, and/or weighted mode) of the component scores produced by any combination of the score techniques of the present disclosure.

In some embodiments a two-dimensional molecular graph of the initial compound in state t docked to the environment of the target macromolecule is inputted into a model, and responsive to this input, the model provides, as output, a corresponding plurality of interaction features for the complex the initial compound in state t docked to the environment of the target macromolecule as disclosed in International Patent Application No. PCT/US24/24456, entitled “Systems and Methods for Discovering Compounds Using Causal Inference,” filed Apr. 12, 2024, which is hereby incorporated by reference. The interaction features identified by the model can be used, at least in part, to determine a score for the initial compound in state t that is evaluated against the compound exit criterion of element 358 of FIG. 4B. In some embodiments, such a model is a graph neural network model, a neural network (e.g., a multi-layer perceptron, a fully connected neural network, a partially connected neural network, etc.), a support vector machine, a Naive Bayes algorithm, a nearest neighbor algorithm, a boosted trees algorithm (e.g., XGBoost, LightGBM), a random forest algorithm, a decision tree algorithm, a logistic regression algorithm, a linear model, a linear regression algorithm, and/or any combination thereof. Various other model architectures are possible for use in obtaining, for an initial compound in state t docked to the environment of the target macromolecule, a corresponding plurality of interaction features for the complex formed between the initial compound in state t docked to the environment of the target macromolecule, as will be apparent to one skilled in the art. In some such embodiments, the model is trained as disclosed in International Patent Application No. PCT/US24/24456, entitled “Systems and Methods for Discovering Compounds Using Causal Inference,” filed Apr. 12, 2024, which is hereby incorporated by reference.

Alternatively or additionally, when the score comprises an individual treatment score calculated as a dot product of an interaction feature vector and corresponding average treatment effects (ATEs) of the respective interaction features as disclosed in International Patent Application No. PCT/US24/24456, entitled “Systems and Methods for Discovering Compounds Using Causal Inference,” filed Apr. 12, 2024, which is hereby incorporated by reference, the initial compound in state t fails to satisfy the criterion when the individual treatment score is greater than a threshold value (e.g., greater than −1, greater than −0.5, greater than −0.1, greater than 0, etc.). In general, because the individual treatment score is calculated using the ATEs of individual interaction features, and because ATEs are representative of the Gibbs free energy of a particular conformation of the initial compound in state t interacting with the environment 154 of the target macromolecule 170, higher individual treatment scores are predictive of poor overall binding affinity or specificity.

In accordance with element 358 of FIG. 4B, (ix) elements (ii), (iii), (iv), (v), (vi), (vii), and (viii) are repeated until a compound exit criterion (e.g., the compound exit criterion comprises a molecular weight, a molecular weight range, a log p, or a log p range) is satisfied by the initial compound in state t, thereby forming a plurality of states for the experience.

In some implementations, satisfaction of the compound exit criterion is dependent on the type of score calculated. For instance, when the score is an absolute count of interaction features causal for binding, as disclosed in International Patent Application No. PCT/US24/24456, entitled “Systems and Methods for Discovering Compounds Using Causal Inference,” filed Apr. 12, 2024, which is hereby incorporated by reference, the initial compound in state t fails to satisfy the compound exit criterion when the absolute count is less than a threshold number of interaction features deemed to be sufficient for potent binding (e.g., less than 100, less than 50, less than 20, less than 10, etc.).

In some embodiments, the compound exit criterion is determined based on a predetermined hypothesis or prior.

In some embodiments, the compound exit criterion is determined based on one or more predetermined parameters known to be associated, highly causal, or necessary with a particular property relevant to interaction (e.g., binding potency, selectivity, ADME properties, toxicity, etc.). Predetermined parameters can be obtained from literature, published data, and/or experimental results. For instance, in some implementations, cutoff thresholds for ADME properties are determined based on outcomes of historical data on other molecules.

In some embodiments, the compound exit criterion is determined based on one or more parameters for a control molecule known to exhibit target properties. For instance, in some implementations, the compound exit criterion is determined by identifying one or more lead candidates or tool compounds that have been observed to exhibit target levels of binding, such as ADME properties, and/or drug-likeness. A lead candidate or tool compound is scored, using any one or more of the scoring methods disclosed above. The values obtained from the scoring methods are then used as a baseline threshold to establish the compound exit criterion for further assessment of other compounds. In some embodiments, a value obtained for a lead compound or tool compound is used to establish the compound exit criterion without alteration. Alternatively, in some embodiments, a value obtained for a lead compound or tool compound is used to adjust the compound exit criterion in order to establish the criterion value (e.g., to encourage identification of compounds having improved performance over the control compounds).

In some embodiments, the initial compound in state t is assigned a terminal positive reward when the compound exit criterion is satisfied.

In some embodiments, the initial compound in state t is assigned a terminal negative reward when the compound exit criterion is satisfied. In some embodiments, (ii), (iii), (iv), (v), (vi), (vii), and (viii) is repeated at least 2, at least 3, at least 4, at least 5, at least 6, at least 7, at least 8, or at least 9 times.

In some embodiments, a compound satisfies the compound exit criterion when the compound satisfies the requirements of Lipinski's Rule of Five, Veber's rules, the Ghose filter, the Egan filter, or Muegge's rule described in blocks 618-622 above.

In some embodiments, the compound exit criterion is satisfied by either a negative condition of the initial compound in state t (e.g., the initial compound in state t exceeds a threshold molecular weight, exceeds a threshold total number of hydrogen bond donors, exceeds a threshold total number of hydrogen bond acceptors, exceeds a threshold number of aromatic rings, exceeds a threshold total polar surface area, etc.) or a positive condition of the initial compound in state t (e.g., achieves a score in 356 that satisfies a threshold condition, satisfies the requirements of Lipinski's Rule of Five, Veber's rules, the Ghose filter, the Egan filter, or Muegge's rule described herein, etc.). When the initial compound in state t has the positive condition, a terminal positive reward is assigned to the initial compound in state t and the (ix) repeating is optionally terminated. When the initial compound in state t has the negative condition, a terminal negative reward is assigned to the initial compound in state t and the (ix) repeating is optionally terminated.

Referring to element 408 of FIG. 4B, even in instances where a terminal condition has been reached for a given experience, the initial compound at state t=0 may be used in another experience. Since the molecular reaction and reactant at each state of the experience is separately sampled from probability distributions, the use of the same initial compound at state t=0 in several different instances will lead to different derived compounds 186. Accordingly, in some embodiments in accordance with element 408 of FIG. 4B, the same selected initial compound (from state t=0) is used in 2, 3, 4, 5, 6, 7, 8, 9, 10, 11, 12, 13, 14, 15 or more, 20 or more, 25 or more, 50 or more, or 100 or more different experiences resulting in 2, 3, 4, 5, 6, 7, 8, 9, 10, 11, 12, 13, 14, 15 or more, 20 or more, 25 or more, 50 or more, or 100 or more different derived compounds 180. Thus, according to element 408 of FIG. 4B, if the selected initial compound (of state t=0) has been used in less a threshold number of different experiences, a new experience at a new state t=0 begins and process control returns to element 346 of FIG. 4A to reselect a molecular reaction for the initial compound at state t=0. Process control jumps to element 346 because the probability distribution of the molecular reactions for the initial compound in state t=0 is already available from the prior experience using the same initial compound in state t=0.

On the other hand, if the selected initial compound (of state t=0) has been used in less a threshold number of different experiences, process control goes to element 410 of FIG. 4C. In accordance with element 410, a determination is made as to whether a sufficient number of experiences have been generated to update the parameters of the parent model and the child model. If not, process control returns to block 342 to begin a new experience with a new initial compound. If a sufficient number of experiences have been evaluated then the parameters of the parent and child model can be updated. To update the parent and child models what is needed is the initial compound in each of the states of the experience, the final derived compound, and some metric for the activity of each such compound against the target macromolecule. In some embodiments, the metric for the activity of each such compound against the target macromolecule is determined by one or more physics model or other scores (e.g., described in element 356 above).

In some embodiments, one or more dimension reduction techniques are applied to one or more geometric representations and/or one or more attribute values for a respective interaction feature.

In some embodiments, a dimension reduction reduces the dimensionality of a respective interaction feature from a first number of dimensions to a second number of dimensions. In some implementations, the starting number of dimensions varies between interaction features (e.g., a first interaction feature in a plurality of interaction features has the same or different number of starting dimensions as a second interaction feature in the plurality of interaction features). In some embodiments, the second number of dimensions after dimension reduction is the same or different for each interaction feature in a plurality of interaction features. For example, in some implementations, each respective interaction feature in a plurality of interaction features has a dimensionality of 1 after transformation.

In some embodiments, the dimension reduction is a principal components algorithm, a random projection algorithm, an independent component analysis algorithm, a feature selection method, a factor analysis algorithm, Sammon mapping, curvilinear components analysis, a stochastic neighbor embedding (SNE) algorithm, an Isomap algorithm, a maximum variance unfolding algorithm, a locally linear embedding algorithm, a t-SNE algorithm, a non-negative matrix factorization algorithm, a kernel principal component analysis algorithm, a graph-based kernel principal component analysis algorithm, a linear discriminant analysis algorithm, a generalized discriminant analysis algorithm, a uniform manifold approximation and projection (UMAP) algorithm, a LargeVis algorithm, a Laplacian Eigenmap algorithm, or a Fisher's linear discriminant analysis algorithm. See, for example, Fodor, 2002, “A survey of dimension reduction techniques,” Center for Applied Scientific Computing, Lawrence Livermore National, Technical Report UCRL-ID-148494; Cunningham, 2007, “Dimension Reduction,” University College Dublin, Technical Report UCD-CSI-2007-7, Zahorian et al., 2011, “Nonlinear Dimensionality Reduction Methods for Use with Automatic Speech Recognition,” Speech Technologies. doi:10.5772/16863. ISBN 978-953-307-996-7; and Lakshmi et al., 2016, “2016 IEEE 6th International Conference on Advanced Computing (IACC),” pp. 31-34. doi:10.1109/IACC.2016.16, ISBN 978-1-4673-8286-1, each of which is hereby incorporated by reference.

In some implementations, a geometric representation and/or an attribute value for a respective interaction feature is represented in scalar or binary values. In some implementations, upon application of a transformation to a respective interaction feature, the geometric representation and/or attribute value is further transformed from scalar values to binary values (e.g., 0 or 1). An example of an interaction feature vector for a corresponding candidate molecule, where the geometric representations and/or attribute values for each interaction feature in the interaction feature vector is binarized to zeros and ones, is illustrated in FIG. 7.

In some embodiments, a derived compound 180 in the corresponding plurality of derived compounds requires at least two, at least three, or at least four different molecular reactions in the plurality of molecular reactions to be synthesized from an initial compound in state t=0 used by the method to construct the derived compound.

In some embodiments, a derived compound 186 in the corresponding plurality of derived compounds requires at least 1, at least 2, at least 3, at least 4, at least 5, or at least 10 molecular reactions in the plurality of molecular reactions to be synthesized from an initial compound in state t=0 used by the method to construct the derived compound. In some embodiments, a derived compound 186 in the corresponding plurality of derived compounds requires no more than 20, no more than 10, no more than 5, or no more than 2 molecular reactions in the plurality of molecular reactions to be synthesized from an initial compound in state t=0 used by the method to construct the derived compound. In some embodiments, a derived compound 186 in the corresponding plurality of derived compounds requires from 1 to 5, from 2 to 10, or from 5 to 20 molecular reactions in the plurality of molecular reactions to be synthesized from an initial compound in state t=0 used by the method to construct the derived compound. In some embodiments, a derived compound in the corresponding plurality of derived compounds requires another range of molecular reactions, starting no lower than 1 molecular reaction and ending no higher than 20 molecular reactions, to be synthesized from an initial compound in state t=0 used by the method to construct the derived compound.

In some embodiments, the plurality of molecular reactions that are evaluated by the parent model (e.g., in element 344 at a given state t) comprises 3, 4, 5, 6, 7, 8, 9, 10, 20, 30, 40, 50, 60, 70, 80, 90, or 100 or more molecular reactions.

In some embodiments, the method further comprises masking those molecular reactions in the plurality of molecular reactions that are incompatible with an exit vector in an initial compound (e.g., before execution of element 344 for a given state t of a given experience). Such a filtering step improves computational efficiency of the parent model since fewer molecular reactions need to be evaluated by the parent model. This filtering step is illustrated as element 406 of FIG. 4A as described above.

In some embodiments, the plurality of experiences that are determined is twenty or more experiences representing 20 or more initial compounds in the plurality of initial compounds. In such an embodiment, when 20 experiences representing 20 initial compounds, process control in block 410 of FIG. 4C passes to element 386 and 390, discussed in further detail below, where the parent and child models are updated. Of course, the number 20 is given as just an example. Moreover, as further explained in block 408 above, any given compound selected from among the initial compounds to initiate one experience, may in fact be used in any number of other experiences as well. Thus, in some embodiments, while 20 experiences will likely represent 20 different derived compounds 186, it may represent fewer than 20 different compounds from the plurality of initial compounds. In some embodiments, the plurality of experiences that are collected before turning process control to elements 686 and 390 is more than 20, 30, 40, 50, 60, 70, 80, 90, or 100 experiences. In some embodiments, the plurality of experiences that are collected before turning process control to elements 386 and 390 is more than 200, 300, 400, 500, 600, 700, 800, 900, or 1000 experiences. In some embodiments, the plurality of experiences that are collected before turning process control to elements 386 and 390 is more than 2000, 3000, 4000, 5000, 6000, 7000, 8000, 9000, or 10,000 experiences. In some embodiments, the plurality of experiences that are collected before turning process control to elements 386 and 390 is more than 20,000, 30,000, 40,000, 50,000, 60,000, 70,000, 80,000, 90,000, or 100,000 experiences. In some embodiments, the plurality of experiences that are collected before turning process control to elements 386 and 390 is more than 1×10⁶, 1×10⁷, or 1×10⁸ experiences.

In some embodiments, the plurality of experiences that are collected before turning process control to elements 386 and 390 represents more than 20, 30, 40, 50, 60, 70, 80, 90, or 100 different derived compounds. In some embodiments, the plurality of experiences that are collected before turning process control to elements 386 and 390 represents more than 200, 300, 400, 500, 600, 700, 800, 900, or 1000 different derived compounds. In some embodiments, the plurality of experiences that are collected before turning process control to elements 386 and 390 represents more than 2000, 3000, 4000, 5000, 6000, 7000, 8000, 9000, or 10,000 different derived compounds. In some embodiments, the plurality of experiences that are collected before turning process control to elements 386 and 390 represents more than 20,000, 30,000, 40,000, 50,000, 60,000, 70,000, 80,000, 90,000, or 100,000 different derived compounds. In some embodiments, the plurality of experiences that are collected before turning process control to elements 386 and 390 represents more than 1×10⁶, 1×10⁷, or 1×10⁸ different derived compounds.

Block 266. Referring to block 266, in some embodiments, the reinforcement learning process further comprises: ii) updating a second plurality of parameters associated with the parent model in accordance with a first surrogate objective calculated using the plurality of experiences (element 386 of FIG. 3C), iii) updating a third plurality of parameters associated with a child model in accordance with a second surrogate objective using the plurality of experiences (element 390 of FIG. 3C); iv) repeating the generating of block 264 of FIG. 2F i), updating of element 386 of FIG. 4C ii), and updating of element 390 of FIG. 4C iii) of until a threshold convergence criterion is satisfied (element 411 of FIG. 3C).

In a first nonlimiting example of element 386 of FIG. 4C, the parent model is updated in accordance with a first surrogate objective calculated using the plurality of experiences. In some such embodiments, the first surrogate objective is a first trust region method. In some such embodiments, the first trust region method comprises:

$${maximize}_{θ} {\hat{𝔼}}_{t} [ \frac{{π}_{θ} ( {a}_{t} \vert {s}_{t} )}{{π}_{{θ}_{old}} ( {a}_{t} \vert {s}_{t} )} {\hat{A}}_{t} ]$$

$$subject to {\hat{𝔼}}_{t} [ KL [ {π}_{{θ}_{old}} ( \cdot \vert {s}_{t} ) , {π}_{θ} ( \cdot \vert {s}_{t} ) ] ] \leq δ$$

where,
[symbol] ₜ is an empirical average taken over the plurality of states for an experience in the plurality of experiences by averaging

$$[ \frac{{π}_{θ} ( {a}_{t} \vert {s}_{t} )}{{π}_{{θ}_{old}} ( {a}_{t} \vert {s}_{t} )} {\hat{A}}_{t} ]$$

for each state t in the plurality of states for the experience,
θ_{old} is the first plurality of parameters prior to the updating of element 386,
θ is the first plurality of parameters upon performing the updating of element 386,
π_{θ}(aₜ|sₜ) is the probability assigned to each respective molecular reaction in the plurality of molecular reactions by the parent model for the complex of state t using 0,
θ_{θ}_{_{old}} (aₜ|sₜ) is the probability assigned to each respective molecular reaction in the plurality of molecular reactions by the parent model at state t using θ_{old},
aₜ is the molecular reaction in the plurality of molecular reactions selected for state t,
sₜ is the initial compound in state t,

$${\hat{A}}_{t} = {δ}_{t} + ( γλ ) {δ}_{t + 1} + … + … + {( γλ )}^{T - t + 1} {δ}_{T - 1} ,$$

γ is a scalar between 0 and 1,
λ is a smoothing parameter,
δₜ is a temporal difference error at state t that represents a difference between (i) a predicted score for the initial compound in state t (ii) and the actual score for the initial compound in state t, plus an estimated score for the initial compound in state t+1,
T is the number of states in the experience,
KL[π_{θ}_{_{old}}(⋅|sₜ), π_{θ}(⋅|sₜ)] is a Kullback-Leibler (KL) divergence between the parent model with θ and the parent model with θ_{old}, and
δ is a maximum allowable KL divergence.

In some embodiments, δₜ has the form:

δₜ=rₜ+γV(sₜ₊₁)−V(sₜ)

where,
rₜ is the score for state t,

$$V ( {s}_{t + 1} ) = {𝔼}_{π} [ {\sum }_{k = 0}^{∞} {γ}^{k} {r}_{t + 1 + k} \vert {s}_{t + 1} ] ,$$

$$V ( {s}_{t} ) = {𝔼}_{π} [ {\sum }_{k = 0}^{∞} {γ}^{k} {r}_{t + k} \vert {s}_{t} ] ,$$

rₜ₊₁₊ₖ is the score for state t+1+k, and
rₜ₊ₖ is the score for state t+k.

In some embodiments, the first trust region method updates θ_{old} to θ using an aggregate of [symbol] ₜ across each experience in the plurality of experiences. More details of such a trust region method are disclosed in Schulman et al., “Proximal Policy Optimization Algorithms,” arXiv:1707.06347v2 [cs.LG]28 Aug. 2017, which is hereby incorporated by reference.

In a second nonlimiting example of element 386 of FIG. 4C, the parent model is updated in accordance with a clipped surrogate objective. In some such embodiments, the clipped surrogate objective comprises:

$${L}^{CLIP} ( θ ) = {\hat{𝔼}}_{t} [ min ( {r}_{t} ( θ ) {\hat{A}}_{t} , clip ( {r}_{t} ( θ ) , 1 - ϵ , 1 + ϵ ) {\hat{A}}_{t} ) ]$$

where,
[symbol] ₜ is an expectation taken over the plurality of states for an experience in the plurality of experiences,
θ is the first plurality of parameters upon performing the updating of element 386,

$${r}_{t} ( θ ) = \frac{{π}_{θ} ( {a}_{t} \vert {s}_{t} )}{{π}_{{θ}_{old}} ( {a}_{t} \vert {s}_{t} )} ,$$

π_{θ}(aₜ|sₜ) is the probability assigned to each respective molecular reaction in the plurality of molecular reactions by the parent model for the complex of state t using θ,
π_{θ}_{_{old}}(aₜ|sₜ) is the probability assigned to each respective molecular reaction in the plurality of molecular reactions by the parent model at state t using θ_{old},

$${\hat{A}}_{t} = {δ}_{t} + ( γλ ) {δ}_{t + 1} + … + … + {( γλ )}^{T - t + 1} {δ}_{T - 1} ,$$

γ is a scalar between 0 and 1,
λ is a smoothing parameter,
δₜ is a temporal difference error at state t that represents a difference between (i) a predicted score for the initial compound in state t (ii) and the actual score for the initial compound in state t, plus an estimated score for the initial compound in state t+1,
T is the number of states in the experience, and
clip(rₜ(θ),1−ϵ, 1+ϵ) is a clipped version of rₜ(θ) bounded within the range 1−ϵ, 1+ϵ.

In some embodiments, the clipped surrogate objective updates θ_{old} to θ using an aggregate of [symbol] ₜ across each experience in the plurality of experiences. More details of such a clipped surrogate objective are disclosed in Schulman et al., “Proximal Policy Optimization Algorithms,” arXiv:1707.06347v2 [cs.LG]28 Aug. 2017, which is hereby incorporated by reference.

Referring to element 390 of FIG. 4C, in some embodiments the third plurality of parameters of the child model is updated in accordance with a second surrogate objective using the plurality of experiences. In some embodiments, the second surrogate objective is a trust region method or a clipped surrogate objective described in conjunction with element 386 of FIG. 4C above and/or further described in Schulman et al., “Proximal Policy Optimization Algorithms,” arXiv:1707.06347v2 [cs.LG]28 Aug. 2017, which is hereby incorporated by reference.

In some embodiments, the generating 264, updating 386, and updating 390 is repeated at least 2, at least 3, at least 4, at least 5, at least 10, at least 20, at least 50, or at least 100 times using at least 2, at least 3, at least 4, at least 5, at least 10, at least 20, at least 50, or at least 100 different initial compounds thereby deriving at least 2, at least 3, at least 4, at least 5, at least 10, at least 20, at least 50, or at least 100 derived compounds. In some embodiments, the generating 264, updating 386, and updating 390 is repeated no more than 200, no more than 100, no more than 50, no more than 10, or no more than 5 times until a threshold convergence criterion is satisfied. In some embodiments, the generating 264, updating 386, and updating 390 is repeated from 2 to 10, from 5 to 50, from 30 to 100, or from 100 to 200 times until a threshold convergence criterion is satisfied. In some embodiments, the generating 265, updating 386, and updating 390 is repeated is repeated a number of times that falls within another range starting no lower than 2 times and ending no higher than 1×10¹⁰ times prior to satisfying a threshold convergence criterion.

In some embodiments, the threshold convergence criterion is a gradient norm threshold. In such embodiments the threshold convergence criterion is satisfied when the norm of a gradient of the objective function (e.g., expected reward) of the parent model with respect to parent model parameters (second plurality of parameters) and/or the child model with respect to the child model parameters (third plurality of parameters) falls below a predefined threshold (e.g., 10⁻³ or 10⁻⁴) indicating that changes to the second plurality of parameters of the parent model are becoming negligible, suggesting that the policy is approaching a local optimum.

In some embodiments, the threshold convergence criterion is an improvement in expected reward in which the threshold convergence criterion is satisfied when the improvement in the expected reward for the parent model and/or child model over a certain number of iterations (412—No of FIG. 4C) is below a specified threshold. This can be measured by average the expected reward of the parent model and/or child model over recent episodes (e.g., each instance of 412—No of FIG. 4C is an example of beginning a new episode). In some such embodiments, a difference of ϵ=10⁻² or lower, over a set number of episodes (e.g., 2, 3, 4, 5, 6, 7, 8, 9, 10) is a suitable threshold.

In some embodiments, the threshold convergence criterion is a maximum number of iterations (412—No of FIG. 4C). For instance in some embodiments, the threshold convergence criterion is satisfied when the generating 264, updating 386, and updating 390 has been repeated 2, 3, 4, 5, 10, 20, 50, or 100 times. In some embodiments, the threshold convergence criterion is satisfied when the generating 264, updating 386, and updating 390 has been repeated 200, 100, 50, 10, or 5 times. In some embodiments, the threshold convergence criterion is satisfied when the generating 264, updating 386, and updating 390 has been repeated between 2 to 10, between 5 to 50, between 30 to 100, or between 100 to 200 times. In some embodiments, the threshold convergence criterion is satisfied when the generating 264, updating 386, and updating 390 has been repeated a number of times that falls within another range starting no lower than 2 times and ending no higher than 1×10¹⁰ times.

In some embodiments, the threshold convergence criterion is a metric for policy stability (e.g., the stability of the first and/or second plurality of parameters) under which the threshold convergence criterion is satisfied when a divergence between successive policies (e.g., divergence between the first and/or second plurality of parameters in successive repetitions of the generating 264, updating 386, and updating 390 (e.g., measured using a distance metric like KL-divergence) becomes small (e.g., a KL-divergence of less than 0.01).

Block 268. Block 268 provides a summary FIG. 4, which has been described above. In some embodiments, an experience in the plurality of experiences is generated (block 264 of FIG. 2F, described above) by (a) initializing the experience to state t=0 (element 342 of FIG. 4A, described above). Next, (b) inputting a complex of state t, in two or three dimensions, of the initial compound in state t interacting with the environment of the target macromolecule into the parent model. The parent model evaluates a first exit vector of the initial compound in state t against the plurality of molecular reactions, thereby assigning a corresponding probability to each respective molecular reaction in the plurality of molecular reactions for state t (element 344 of FIG. 4A, described above). Next, (c) selecting a molecular reaction in the plurality of molecular reactions through a sampling of the plurality of molecular reactions using the corresponding probability assigned to each molecular reaction in the plurality of molecular reactions for state t (element 346 of FIG. 4A, described above). Next, (d) inputting the complex of state t into the child model, where the child model evaluates the initial compound in state t against each reactant in a corresponding plurality of reactants available for reaction using the molecular reaction selected for state t, thereby assigning a corresponding probability to each respective reactant in the corresponding plurality of reactants for state t. (element 348 of FIG. 4A, described above). Next, (e) selecting a reactant in the corresponding plurality of reactants, through a sampling of the corresponding plurality of reactants using the corresponding probability assigned to each reactant in the corresponding plurality of reactants for state t (element 350 of FIG. 4B, described above). Next, (f) advancing state t to state t+1 (element 352 of FIG. 4A, described above). Next, (g) forming the initial compound in state t through an in silico reaction of the initial compound in state t−1 in accordance with the selected molecular reaction and the selected reactant of state t (element 354 of FIG. 4B, described above). Next, (h) determining a score for the initial compound in state t interacting with the environment of the target macromolecule by inputting the initial compound in state t interacting with the environment of the target macromolecule into a physics model (element 356 of FIG. 4B, described above). The (b) inputting, (c) selecting, (d) inputting, (e) selecting, (f) advancing, (g) forming, and (h) determining is repeated until a compound exit criterion is satisfied by the initial compound in state t (element 358 of FIG. 4B, described above) thereby forming a plurality of states for the experience.

Block 270. Referring to block 270, in some embodiments, the identifying comprises in silico screening of a database of compounds using the on the target macromolecule binding hypothesis as a selection criterion. Block 270 is known as design space reduction. Here, the target macromolecule binding hypothesis is used to filter large libraries of compounds. Examples of large libraries of compounds that can be screened using the target macromolecule binding hypothesis in accordance with block 270 include, but are not limited to, MCULE (Kiss et al., 2012, “Http://Mcule.Com: A Public Web Service for Drug Discovery,” J. Cheminformatics 4 (1), p. 17.) and ENAMINE (Irwin et al., 2016, “Docking Screens for Novel Ligands Conferring New Biology,” J. Med. Chem. 59 (9), pp. 4103-4120). In some embodiments, the database of compounds that is screened in accordance with block 270 comprises 10,000 or more compounds, 100,000 or more compounds, 1×10⁶ or more compounds, 1×10⁷ or more compounds, 1×10⁸ or more compounds, 1×10⁹ or more compounds, 1×10¹⁰ or more compounds, 1×10¹¹ or more compounds, 1×10¹² or more compounds, 1×10¹³ or more compounds, 1×10¹⁴ or more compounds, 1×10¹⁵ or more compounds, 1×10¹⁶ or more compounds, or 1×10¹⁷ or more compounds,

F) Testing the Plurality of Derived Compounds for Activity Against the Target Macromolecule, Thereby Identifying One or More Compounds that Exhibit the Threshold Activity with Respect to the Target Macromolecule.

Block 272. Referring to block 272, the plurality of derived compounds is tested for activity against the target macromolecule, thereby identifying one or more compounds that exhibit the threshold activity with respect to the target macromolecule.

In accordance with block 394 of FIG. 3, described in further detail below in conjunction with block 272, the plurality of derived compounds 180, from the plurality of experiences, is tested in an assay (e.g., a wet lab assay) for activity against the target macromolecule, thereby identifying one or more derived compounds that exhibit the threshold activity with respect to the target macromolecule.

In some embodiments, the plurality of derived compounds is 2, 3, 4, 5, 6, 7, 8, 9, or 10 or more derived compounds. In some embodiments, the plurality of derived compounds is at least 20, 30, 40, 50, 60, 70, 80, 90, or 100 derived compounds. In some embodiments, the plurality of derived compounds is at least 200, 300, 400, 500, 600, 700, 800, 900, or 1000 derived compounds. In some embodiments, the plurality of derived compounds is between 5 and 1000, 10 and 2000, or 20 and 3000 derived compounds. In some embodiments, the plurality of derived compounds is more than two derived compounds and less than 100, 500, or 1000 derived compounds.

Blocks 274-278. Referring to block 274, in some embodiments, each compound in the one or more compounds is an organic compound having a molecular weight of less than 50 Daltons, less than 100 Daltons, less than 150 Daltons, less than 200 Daltons, less than 250 Daltons, less than 300 Daltons, less than 400 Daltons, less than 500 Daltons, or less than 1000 Daltons. Referring to block 276, in some embodiments, each compound in the one or more compounds is an organic compound having a molecular weight of between 500 Daltons and 1000 Daltons. Block 278. Referring to block 278, in some embodiments, each compound in the one or more compounds is an organic compound having a molecular weight of between 400 Daltons and 10000 Daltons.

In some embodiments, each compound in the one or more compounds is an organic compound having a molecular weight of less than 50 Daltons, less than 100 Daltons, less than 150 Daltons, less than 200 Daltons, less than 250 Daltons, less than 300 Daltons, less than 400 Daltons, less than 500 Dalton, or less than 1000 Daltons.

In some embodiments, a compound in the one or more compounds has a molecular weight of at least 100, at least 500, at least 1000, at least 2000, at least 5000, or at least 10,000 Daltons. In some embodiments, a compound in the one or more compounds has a molecular weight of no more than 20,000, no more than 10,000, no more than 8000, no more than 6000, no more than 4000, no more than 2000, no more than 1000, or no more than 500 Daltons. In some embodiments, a compound in the one or more compounds has a molecular weight of from 100 to 500, from 500 to 2000, from 1000 to 8000, or from 5000 to 20,000 Daltons. In some embodiments, a compound in the one or more compounds has a molecular weight that falls within another range starting no lower than 100 Daltons and ending no higher than 20,000 Daltons. However, some embodiments of the disclosed systems and methods have no limitation on the size of the one or more compounds.

Block 280. Referring to block 280, in some embodiments, a compound in the one or more compounds satisfies any two or more, any three or more, or all four of the conditions: (i) not more than five hydrogen bond donors, (ii) not more than ten hydrogen bond acceptors, (iii) a molecular weight under 500 Daltons, and (iv) a Log P under 5. See, Lipinski, 1997, Adv. Drug Del. Rev. 23, 3, which is hereby incorporated herein by reference in its entirety. In some embodiments, a compound in the one or more compounds satisfies one or more criteria in addition to Lipinski's Rule of Five. For example, in some embodiments, a compound in the one or more compounds has five or fewer aromatic rings, four or fewer aromatic rings, three or fewer aromatic rings, or two or fewer aromatic rings.

In some embodiments, a compound in the one or more compounds satisfies Veber's rules: (i) the number of rotatable bonds (≤10) and the total polar surface area (TPSA) (≤140 Å²). In some embodiments, each derived compound 180 satisfies Veber's rules. See, Kralj et al., “Molecular Filters in Medicinal Chemistry,” Encyclopedia 2023, 3, 501-511, and Veber et al., 2002, “Molecular Properties That Influence the Oral Bioavailability of Drug Candidates,” J. Med. Chem. 45, 2615-2623, each of which is hereby incorporated by reference.

In some alternative embodiments, a compound in the one or more compounds satisfies a Ghose filter: log P (octanol-water partition coefficient), molecular weight (160-480 Da), molar refractivity (40-130), and the number of atoms (20-70). In some embodiments, each derived compound 180 satisfies a Ghose filter. See, Kralj et al., “Molecular Filters in Medicinal Chemistry,” Encyclopedia 2023, 3, 501-511, and Ghose et al., 1999, “A Knowledge-Based Approach in Designing Combinatorial or Medicinal Chemistry Libraries for Drug Discovery, 1. A Qualitative and Quantitative Characterization of Known Drug Databases,” J. Comb. Chem. 1, pp. 55-68, each of which is hereby incorporated by reference.

In some embodiments, a compound in the one or more compounds satisfies Egan's filter: compound has a log P≤5.88 and a total polar surface area of ≤131.6 Å². In some embodiments, each derived compound 180 satisfies Egan's filter. See, Egan et al., 2000 “Prediction of Drug Absorption Using Multivariate Statistics,” J. Med. Chem. 43, pp. 3867-3877 each of which is hereby incorporated by reference.

In some embodiments, a compound in the one or more compounds satisfies Muegge's rule: molecular weight (200-600 Daltons), log P (−2 to 5), PSA≤150, number of rings (≤7), and number of rotatable bonds (≤15), number of carbons >4, number of heteroatoms >1, number of hydrogen bond donors ≤5. In some alternative embodiments, each derived compound satisfies Muegge's rule. See, Velez et al, 2022, “Theoretical calculations and analysis method of the physicochemical properties of phytochemicals to predict gastrointestinal absorption,” Int. J. Plant Biol. 13(2), pp. 163-179, which is hereby incorporated by reference.

Block 282-286. Referring to block 282, in some embodiments, the threshold activity with respect to the target macromolecule is an IC50, EC50, Kd, KI, hill coefficient (nH), negative logarithm of EC₅₀(pEC50), association rate constant (Kon), or disassociation rate constant (Koff), for a compound with respect to the target macromolecule. Accordingly, in some embodiments, one or more compounds identified using the systems and methods of the present disclosure are tested in a wet lab assay to determine whether they have potency against a therapeutic target. In some embodiments, the goal of such an assay is to determine a binding coefficient of the compound to the target macromolecule 170. In some such embodiments, the binding coefficient is an IC₅₀, EC₅₀, Kd, KI, or pKI for the compound with respect to the target macromolecule 170. IC₅₀, EC₅₀, Kd, KI, and pKI, as well as suitable wet lab assays are generally described in Huser ed., 2006, High-Throughput-Screening in Drug Discovery, Methods and Principles in Medicinal Chemistry 35; and Chen ed., 2019, A Practical Guide to Assay Development and High-Throughput Screening in Drug Discovery, each of which is hereby incorporated by reference.

In some embodiments a compound has a threshold activity with respect to the target macromolecule when the compound has an IC₅₀, EC₅₀, Kd, or KI of less than 1 molar, less than 1 millimolar, less than 100 micromolar, less than 10 micromolar, less than 1 micromolar, less than 100 nanomolar, less than 10 nanomolar, or less than 1 nanomolar.

Referring to block 284, in some embodiments, the testing tests the plurality of derived compounds using a quantum mechanics algorithm.

In some embodiments, the testing tests the plurality of derived compounds using a molecular dynamics simulation. Molecular dynamics simulations capture the behavior of proteins and other biomolecules in full atomic detail and at very fine temporal resolution. Such simulations can be used to decipher the functional mechanisms of proteins and other biomolecules, uncover the structural basis for disease, and aid in the design and optimization of small molecules, peptides, and proteins. See, for example, Durrant and McCammon, “Molecular dynamics simulations and drug discovery,” BMC Biology. 2011; 9(1):71; and Hollingsworth and Dror, “Molecular dynamics simulation for all,” Neuron. 2018; 99(6):1129-1143, each of which is hereby incorporated herein by reference in its entirety.

Referring to block 286, in some embodiments, the testing tests the plurality of derived compounds using a wet lab assay. Suitable wet lab assays are generally described in Huser ed., 2006, High-Throughput-Screening in Drug Discovery, Methods and Principles in Medicinal Chemistry 35; and Chen ed., 2019, A Practical Guide to Assay Development and High-Throughput Screening in Drug Discovery, each of which is hereby incorporated by reference.

In some embodiments, the target macromolecule 170 is associated with a condition. In some embodiments, the condition is a disease. In some embodiments, the condition is a cancer, hematologic disorder, autoimmune disease, inflammatory disease, immunological disorder, metabolic disorder, neurological disorder, genetic disorder, psychiatric disorder, gastroenterological disorder, renal disorder, cardiovascular disorder, dermatological disorder, respiratory disorder, viral infection, or other disease or disorder.

In some embodiments the wet lab assay or quantum mechanics algorithm validates a compound identified by the systems and methods of the present disclosure as being a suitable compound for alleviation of the condition. In some such embodiments the compound is used in in vivo assays such as animal models.

In some embodiments, a compound identified by the systems and methods of the present disclosure is combined with one or more excipient and/or one or more pharmaceutically acceptable carrier and/or one or more diluent when administering to an animal model or a human.

Such excipients and/or carriers include all conventional solvents, dispersion media, fillers, solid carriers, coatings, antifungal and antibacterial agents, dermal penetration agents, surfactants, isotonic and absorption agents and the like.

An exemplary carrier is pharmaceutically “acceptable” in the sense of being compatible with the other ingredients of the composition (e.g., the composition comprising the selected compound in the plurality of compounds) and not injurious to a subject. The compound may conveniently be presented in unit dosage form and may be prepared by any of the methods well known in the art of pharmacy. Such methods include bringing into association the compound with the carrier that constitutes one or more accessory ingredients. In general, the compound is prepared by uniformly and intimately bringing into association the compound with liquid carriers or finely divided solid carriers or both.

Exemplary compounds formulated for intravenous, intramuscular or intraperitoneal administration, or a pharmaceutically acceptable salt, solvate or prodrug thereof may be administered by injection or infusion.

In some embodiments, injectables for such use are prepared in conventional forms, either as a liquid solution or suspension or in a solid form suitable for preparation as a solution or suspension in a liquid prior to injection, or as an emulsion. In some embodiments, carriers include, for example, water, saline (e.g., normal saline (NS), phosphate-buffered saline (PBS), balanced saline solution (BSS)), sodium lactate Ringer's solution, dextrose, glycerol, ethanol, and the like; and if desired, minor amounts of auxiliary substances, such as wetting or emulsifying agents, buffers, and the like can be added. Proper fluidity can be maintained, for example, by using a coating such as lecithin, by maintaining the required particle size in the case of dispersion and by using surfactants.

In some embodiments, the compound identified in accordance with block 272 is also suitable for oral administration and presented as discrete units such as capsules, sachets or tablets each containing a predetermined amount of the test chemical compound; as a powder or granules; as a solution or a suspension in an aqueous or non-aqueous liquid; or as an oil-in-water liquid emulsion or a water-in-oil liquid emulsion. In some embodiments, the compound is presented as a bolus, electuary or paste.

In some embodiments, a tablet of the compound is made by compression or molding, optionally with one or more accessory ingredients. In some embodiments, compressed tablets are prepared by compressing in a suitable machine the test chemical compound in a free-flowing form such as a powder or granules, optionally mixed with a binder (e.g., inert diluent, preservative disintegrant, e.g. sodium starch glycolate, cross-linked polyvinyl pyrrolidone, cross-linked sodium carboxymethyl cellulose, surface-active or dispersing agent). In some embodiments, molded tablets are made by molding in a suitable machine a mixture of the powdered compound moistened with an inert liquid diluent. In some embodiments, the tablets are optionally coated or scored and may be formulated so as to provide slow or controlled release of the compound therein using, for example, hydroxypropylmethyl cellulose in varying proportions to provide the desired release profile. In some embodiments, tablets are optionally provided with an enteric coating, to provide release in parts of the gut other than the stomach.

In some embodiments, the compound identified in accordance with block 272 is suitable for topical administration in the mouth including lozenges comprising the active ingredient in a flavored base, usually sucrose and acacia or tragacanth gum; pastilles comprising the active ingredient in an inert basis such as gelatine and glycerin, or sucrose and acacia gum; and mouthwashes comprising the active ingredient in a suitable liquid carrier.

In some embodiments, the compound identified in accordance with block 272 is suitable for topical administration to the skin. In some such instances, the compound is dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. In some embodiments, transdermal patches are used to administer the compound.

In some embodiments, the compound identified in accordance with block 272 is suitable for parenteral administration. In such embodiments, the compound includes aqueous and non-aqueous isotonic sterile injection solutions that contain anti-oxidants, buffers, bactericides and solutes that render the compound isotonic with the blood of the intended recipient; and aqueous and non-aqueous sterile suspensions that include suspending agents and thickening agents. In some embodiments, the compound is presented in unit-dose or multi-dose sealed containers, for example, ampoules and vials, and stored in a freeze-dried (lyophilized) condition requiring only the addition of the sterile liquid carrier, for example water for injections, immediately prior to use. In some embodiments, extemporaneous injection solutions and suspensions are prepared from sterile powders, granules and tablets of the kind previously described.

It should be understood that in addition to the compound particularly mentioned above (e.g., a compound identified in accordance with block 272), the composition or combination of this present disclosure (e.g., a selected compound identified in accordance with block 272) may include other agents conventional in the art having regard to the type of composition or combination in question, for example, those suitable for oral administration may include such further agents as binders, sweeteners, thickeners, flavoring agents disintegrating agents, coating agents, preservatives, lubricants and/or time delay agents. Suitable sweeteners include sucrose, lactose, glucose, aspartame or saccharine. Suitable disintegrating agents include cornstarch, methylcellulose, polyvinylpyrrolidone, xanthan gum, bentonite, alginic acid or agar. Suitable flavoring agents include peppermint oil, oil of wintergreen, cherry, orange or raspberry flavoring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

In some embodiments, the present disclosure informs the selection of one or more human subjects for treatment with the compound identified in accordance with block 272 and/or selection of one or more human subjects for continuation or discontinuation of treatment with the compound.

In some embodiments, the present disclosure informs the dosing amount, duration, and/or frequency of the compound in one or more human subjects for treatment.

In some embodiments, the present disclosure informs the design of a clinical trial, the clinical trial comprising the use of the compound identified in accordance with block 272. In some embodiments, the present disclosure informs the design of an adaptive clinical trial, the adaptive clinical trial comprising the use of the compound.

In some embodiments, the present disclosure further comprises formulating the compound identified in accordance with block 272 for use in a therapy. In some embodiments, this includes formulating the compound with any of the excipients, pharmaceutically acceptable carrier, diluents, or other pharmacological formulations described in the present disclosure or known in the art. In some embodiments, the therapy is to alleviate a condition such as inflammation. In some embodiments the therapy is to alleviate or treat a disease or disorder. In some embodiments the disease or disorder is cancer, a hematologic disorder, an autoimmune disease, an inflammatory disease, an immunological disorder, a metabolic disorder, a neurological disorder, a genetic disorder, a psychiatric disorder, a gastroenterological disorder, a renal disorder, a cardiovascular disorder, a dermatological disorder, a respiratory disorder, a viral infection, or other disease or disorder.

Use Cases. In some embodiments, the systems and methods disclosed herein are advantageously used in any number of applications, including but not limited to hit discovery, hit-to-lead discovery, lead optimization, molecular dynamics simulations, toxicity prediction, potency optimization, selectivity optimization, fitness modeling, drug resistance prediction, personalized medicine, and drug trial design. The following are more details of sample use cases provided for illustrative purposes only that describe some applications of some embodiments of the present disclosure. Other uses may be considered, and the examples provided below are non-limiting and may be subject to variations, omissions, or may contain additional elements.

Hit discovery. Pharmaceutical companies spend millions of dollars on screening compounds to discover new prospective drug leads. Large compound collections are tested to find the small number of compounds that have any interaction with the disease target of interest. Unfortunately, wet lab screening suffers experimental errors and, in addition to the cost and time to perform the assay experiments, the gathering of large screening collections imposes significant challenges through storage constraints, shelf stability, or chemical cost. Even the largest pharmaceutical companies have only between hundreds of thousands to a few millions of compounds, versus the tens of millions of commercially available molecules, and the hundreds of millions, billions, and even trillions of simulate-able molecules. Example of databases of commercially available molecules include MCULE (Kiss et al., 2012, “Http://Mcule.Com: A Public Web Service for Drug Discovery,” J. Cheminformatics 4 (1), p. 17.) and ENAMINE (Irwin et al., 2016, “Docking Screens for Novel Ligands Conferring New Biology,” J. Med. Chem. 59 (9), pp. 4103-4120).

A potentially more efficient alternative to physical experimentation is virtual high throughput screening. In the same manner that physics simulations can help an aerospace engineer to evaluate possible wing designs before a model is physically tested, computational screening of molecules can focus the experimental testing on a small subset of high-likelihood molecules. This may reduce screening cost and time, reduces false negatives, improves success rates, and/or covers a broader swath of chemical space.

In this application, a protein target may be provided as input to the system. A large set of compounds may also be provided in silico. For each compound, the target macromolecule binding hypothesis target macromolecule is used to determine a compound score. The resulting compound scores may be used to rank the compounds, with the best-scoring compounds being most likely to bind the target protein. Optionally, the ranked compounds list is analyzed for clusters of similar compounds; a large cluster may be used as a stronger prediction of compound binding, or compounds may be selected across clusters to ensure diversity in the confirmatory experiments.

Drug resistance prediction. Drug resistance is an inevitable outcome of pharmaceutical use, which puts selection pressure on rapidly dividing and mutating pathogen populations. Drug resistance is seen in such diverse disease agents as viruses (HIV), exogenous microorganisms (MRSA), and disregulated host cells (cancers). Over time, a given medicine will become ineffective, irrespective of whether the medicine is an antibiotic or a chemotherapy. At that point, the intervention can shift to a different medicine that is, hopefully, still potent. In HIV, there are well-known disease progression pathways that are defined by which mutations the virus will accumulate while the patient is being treated.

There is considerable interest in predicting how disease agents adapt to medical intervention. One approach is to characterize which mutations will occur in the disease agent while under treatment. Specifically, the protein target of a medicine needs to mutate so as to avoid binding the drug while simultaneously continuing to bind its natural substrate.

In this application, a set of possible mutations in the target protein may be proposed. For each mutation, the resulting protein shape may be predicted. For each of these mutant protein forms, a target macromolecule binding hypothesis may be formed and the system may be configured to predict a binding affinity for both the natural substrate and the drug. The mutations that cause the protein to no longer bind to the drug but also to continue binding to the natural substrate are candidates for conferring drug resistance. These mutated proteins may be used as targets against which to design drugs, e.g. by using these proteins as inputs to one of these other prediction use cases.

Personalized medicine. Ineffective medicines should not be administered. In addition to the cost and hassle, all medicines have side-effects. Moral and economic considerations make it imperative to give medicines only when the benefits outweigh these harms. It may be important to be able to predict when a medicine will be useful. People differ from one another by a handful of mutations. However, small mutations may have profound effects. When these mutations occur in the disease target's active (orthosteric) or regulatory (allosteric) sites, they can prevent the drug from binding and, therefore, block the activity of the medicine. When a particular person's protein structure is known (or predicted), the system can be configured to predict whether a drug will be effective or the system may be configured to predict when the drug will not work.

For this application, the system may be configured to receive as input the drug's chemical structure and the specific patient's particular expressed protein. The system may be configured to predict binding between the drug and the protein using a target macromolecule binding hypothesis for the particular expressed protein and, if the drug's predicted binding affinity that particular patient's protein structure is too weak to be clinically effective, clinicians or practitioners may prevent that drug from being fruitlessly prescribed for the patient.

Drug trial design. This application generalizes the above personalized medicine use case to the case of patient populations. When the system can predict whether a drug will be effective for a particular patient phenotype, this information can be used to help design clinical trials. By excluding patients whose particular disease targets will not be sufficiently affected by a drug, a clinical trial can achieve statistical power using fewer patients. Fewer patients directly reduces the cost and complexity of clinical trials.

For this application, a user may segment the possible patient population into subpopulations that are characterized by the expression of different proteins (due to, for example, mutations or isoforms). The system may be configured to predict the binding strength of the drug candidate against the different protein types using a target macromolecule binding hypotheses associated with the different protein types. If the predicted binding strength against a particular protein type indicates a necessary drug concentration that falls below the clinically-achievable in-patient concentration (as based on, for example, physical characterization in test tubes, animal models, or healthy volunteers), then the drug candidate is predicted to fail for that protein subpopulation. Patients with that protein may then be excluded from a drug trial.

Simulation. Simulators often measure the binding affinity of a compound to a protein, because the propensity of a compound to stay in a region of the target protein correlates to its binding affinity there. An accurate description of the features governing binding, as exemplified by the disclosed a target macromolecule binding hypotheses, could be used to identify regions and poses that have particularly high or low binding energy. The energetic description can be folded into molecular dynamic simulations to describe the motion of a molecule and the occupancy of the protein binding region. Similarly, stochastic simulators for studying and modeling systems biology could benefit from an accurate prediction of how small changes in compound concentrations impact biological networks.

### Example 1

This example details components of a method for identifying one or more compounds that exhibit a threshold activity with respect to a target macromolecule in accordance with one embodiment of the present disclosure.

The target macromolecule for this example is the transcription factor “signal transducer and activator of transcription 6” (STAT6). STAT6 is the signal mediator of interleukin (IL)-4 and IL-13, promoting an anti-inflammatory process by inducing the development of T helper (Th) 2 lymphocytes and M2 type macrophages. Activation of STAT6 is initiated by binding of IL-4 and IL-13 to their receptors, which leads to the activation of Janus tyrosine kinases (JAKs), which are associated with the cytoplasmic tails of the receptors. STAT6 phosphorylation leads to dimerization followed by translocation to the nucleus where STAT6 regulates gene expression. Stat6 has a challenging active site with few known interactions.

In accordance with block 204, for each respective compound fragment in a plurality of compound fragments, a corresponding plurality of poses of the respective compound fragment against an atomic model of STAT6 was performed thereby constructing a pose set. The atomic model was PDB entry 4Y5U (rcsb.org/structure/4y5u) published in Li et al., 2016, Proc Natl Acad Sci USA 113: 13015-13020, which is hereby incorporated by reference. In this example, the pose set consisted of the top 40 poses for each fragment in the pose set.

The fragments used in this example are disclosed in Table 1 below in SMILES format.

TABLE 1
Fragments used in example 1.

| Compound Fragment (SMILES format) | Compound Fragment (SMILES format) |
|---|---|
| Cc1cccc(c1)C(═O)NN | Cn1cnc2N(C)C(═O)NC(═O)c12 |
| NC1═NCc2ccccc12 | Nc1nc(═O)c2nc[nH]c2N1 |
| [NH2]CC(═O)c1ccc(Br)cc1 | OC(═O)c1ccc(cc1)[N+]([O—])═O |
| Cn1nc(C)cc1CN | Clc1ccc2OC(═O)Nc2c1 |
| NNC(═O)Cc1ccc(F)cc1 | NCCCCC(O)═O |
| [NH2]CCc1cccnc1 | O═C1NC2NC(═O)NC2N1 |
| CN(C)c1ccc(cn1)C(O)═O | Nc1c(C(═O)O)[nH]c(═O)[nH]c1═O |
| [O—]C(═O)[C@@H]1CCCN1C(═O)OCc2ccccc2 | Cn1cnc(C[C@H](N)C(O)═O)c1 |
| NC(═O)c1cccnc1 | Cn1cnc2N(C)C(═O)N(C)C(═O)c12 |
| COC(═O)C(N)Cc1ccccc1 | NC(CCON═C(N)N)C(O)═O |
| N[C@H](CCONC(═N)N)C(O)═O | Nc1cccc(c1)c2ocnc2 |
| NC[C@H](O)c1ccc(O)c(O)c1 | Oc1ccc2[nH]ccc2c1 |
| CC(C)C(═O)Nc1ccc(c(c1)C(F)(F)F)[N+]([O—])═O | NC(═O)c1cccc(N)c1 |
| O═C1CC[C@@H](C(═O)O)N1 | NCC1CCC(CC1)C(O)═O |
| N[C@H](CCCNC(═N)N)C(O)═O | CSCC[C@H](NC(N)═O)C([O—])═O |
| NCC(═O)NCC(═O)NCC(O)═O | CN(C)NC(═O)CCC(O)═O |
| O═C1NC(═O)C2═NNNC2═N1 | NC(═O)c1ccc(O)cc1 |
| Cc1onc(N[S](═O)(═O)c2ccc(N)cc2)c1 | Oc1ccc(c2cccnc12)[N+]([O—])═O |
| C[N+](C)(C)C[C@H](O)CC([O—])═O | CC(═O)N1CCCC1C(O)═O |
| Oc1ccc(cc1O)[N+]([O—])═O | [O—]c1cc(cc(c1[O—])[N+]([O—])═O)[N+]([O—])═O |
| [O—][N+](═O)c1cc2NC(═O)C(═O)Nc2cc1[N+]([O—])═O | O═C(O)C(O)c1cccc(C1)c1 |
| N[S](═O)(═O)c1sc(Cl)cc1 | CNCc1oc(Oc2cccnc2)cc1 |
| CNC(═S)Nc1ccc(Br)cc1Cl | C(N(CC)C(C)═[N])C |
| Cc1cc(N2CCCCCC2)ncn1 | Cc1cccc(NC(═O)CN(C)CC#N)n1 |
| CCC(C)(CN)N1CCOCC1 | CN(C)c1cccc(c1)C(═O)NN |
| C1CCC(C1)NCc2ccc3OCOc3c2 | C1CNC(C1)c2ccc3OCCOc3c2 |
| O═C(CN1CCCCC1)Nc1ccc2c(c1)OCO2 | Nc1[nH]nc(N2CCCC2)c1C#N |
| CC(C)N═c1cccccc1NC(C)C | NC(═N)N1CCCCC1 |
| O═C1[N]c2ccc(NCc3ccccn3)cc2[N]1 | Cc1cc(C(═O)NCC(F)(F)F)no1 |
| Fc1ccc(C2═NNC3═NCCN3C2)cc1F | CC1CCN(CC(═O)Nc2cc(C(C)C)no2)CC1 |
| CC(NC(═O)CCC(═O)c1cccs1)c1cccnc1 | Cn1cccc1CNCCC1═C[N]c2ccccc21 |
| Cc1cc(C)c(C#N)c(NCCCN2CCOCC2)n1 | Cn1c(S)nnc1COc2ccccc2Cl |
| Cn1ccnc1c2sc(N)nc2C | C[N]Cc1nccn1C |
| CC(c1nc(N)nc(N(C)C)n1)N1CCCCCC1 | O═C(Cc1cn2ccccc2n1)Nc3ccccc3 |
| NC(═N)c1cscc1 | C1CCCC(C1)C(═O)[N]CC2CCCO2 |
| NCc1oc(cc1)C(F)(F)F | CC(C1CC1)N(C)Cc1nc2ccccc2c(═O)[nH]1 |
| O═C(C1CCCNC1)N2CCCCC2 | Cc1oc(C)c(c1)C(═O)Nc2ccncc2 |
| COC(═O)c1ccc(CN)cc1 | Cc1nn(C)c(C)c1CC(═O)Nc1ccccn1 |
| NC(═N)c1ccc(cc1)C(F)(F)F | CC(═O)N1C═Cc2ccccc2C1CC(═O)N(C)C |
| CC(C(═O)C1═C[N]c2ccccc21)N1CCCCCC1 | NCC(O)c1ccc(F)cc1 |
| CC(═O)Nc1cccc(CN)c1 | CC(C)c1noc(n1)C2CCCN2 |
| COc1ccc(CN)cc1O | O═C1OCC2CNCCN12 |
| C1COc2cc(NCc3ccncc3)ccc2O1 | NC(═O)C1CCOC1 |
| NC(═N)SCc1ccccc1Cl | ONC(═O)C12CCC(CC1)C2 |
| O═c1[nH]cnc2c1N═C(N1CCCCC1)[N]2 | |
| Cc1oc2ncn(CCCN(C)C)c(═N)c2c1C | |
| CC1CCC(NC(═O)Cn2ccnc2)CC1 | |
| CC(C)(C)c1cc(CC2([N])COC2)no1 | |
| Cc1cccc(C2C[C@@H](O)[C@@H](O)[C@@H]2[N])c1 | |
| O[C@@H]1CNCCOC1 | |

Before screening, any isomers of the compounds of Table 1 were generated, thereby expanding the plurality of compounds fragments used in the screening in accordance with block 204 to those listed in Table 2.

TABLE 2
Fragments used in example 1 (expanded to inc1ude all isomers of Table 1).

| Compound Fragment (SMILES format) | Compound Fragment (SMILES format) |
|---|---|
| C[C@@H](c1cccnc1)NC(═O)CCC(═O)c2cccs2 | CC(C)c1nc(on1)[C@H]2CCC[NH2+]2 |
| c1cc(ccc1C(═[NH2+])N)C(F)(F)F | Cc1cccc(c1)[C@@H]2C[C@H]([C@H]([C@@H]2[NH+])O)O |
| C[C@H](C1CC1)[N@@H+](C)Cc2[nH]c(═O)c3ccccc3n2 | c1cc(c(cc1[C@H]2CN3CC[NH+]═C3N═N2)F)F |
| c1c(cc(c(c1N(═O)═O)[O—])O)N(═O)═O | c1ccc(cc1)COC(═O)N2CCC[C@H]2C(═O)[O—] |
| Cc1cccc(n1)NC(═O)C[N@@](C)CC#N | C1CCN(CC1)C(═[NH2+])N |
| CC(═O)N1C═Cc2ccccc2[C@H]1CC(═O)N(C)C | Cc1cccc(c1)[C@H]2C[C@H]([C@H]([C@@H]2[NH+])O)O |
| CC(C)Nc\1ccccc/c1═N/C(C)C | CC1CC[NH+](CC1)CC(═O)Nc2cc(no2)C(C)C |
| CC(═O)N1CCC[C@H]1C(═O)[O—] | C(CC[NH3+])CC(═O)[O—] |
| c1cc2c(cc[nH]2)cc1O | Cc1c(sc(n1)N)c2nccn2C |
| COC(═O)[C@H](Cc1ccccc1)[NH3+] | C1COC[C@@H]1C(═O)N |
| c1cc2c(cc1NCc3ccncc3)OCCO2 | Cc1cc(nc(c1C#N)NCCC[NH+]2CCOCC2)C |
| c1cc2c(cc1[C@@H]3CCC[NH2+]3)OCCO2 | c1cc(cc(c1)N)c2cnco2 |
| C(CO[NH+]═C(N)N)[C@H](C(═O)[O—])[NH3+] | c1cc2c(cc1C[NH2+]C3CCCC3)OCO2 |
| CSCC[C@@H](C(═O)[O—])NC(═O)N | CC(═O)N1CCC[C@@H]1C(═O)[O—] |
| c1c2c(cc(cIN(═O)═O)N(═O)═O)[nH]c(═O)c(═O)[nH]2 | c1(c([nH]c(═O)[nH]c1═O)C(═O)[O—])N |
| CC(C)(C)c1cc(no1)CC2(COC2)[NH+] | Cc1cc(c(o1)C)C(═O)Nc2ccncc2 |
| C1CCN(CC1)C(═O)[C@@H]2CCC[NH2+]C2 | c1cc2c(ccc(c2nc1)[O—])N(═O)═O |
| C(C[C@H](C(═O)[O—])[NH3+])C[NH+]═C(N)N | c1ccc(c(c1)CSC(═[NH2+])N)Cl |
| COC(═O)[C@@H](Cc1ccccc1)[NH3+] | CNC(═S)Nc1ccc(cc1Cl)Br |
| C[C@@H](C1CC1)[N@@H+](C)Cc2[nH]c(═O)c3ccccc3n2 | C(CO[NH+]═C(N)N)[C@@H](C(═O)[O—])[NH3+] |
| C[C@H](c1nc(nc(n1)N(C)C)N)[NH+]2CCCCCC2 | c1[nH]c2c(n1)C(═O)[N][C@H](N2)[NH3+] |
| Cc1cc(n(n1)C)C[NH3+] | Cn1c(n[nH]c1═S)COc2ccccc2Cl |
| Cc1c(oc2c1c(═[NH2+])n(cn2)CCC[NH+](C)C)C | c1ccc(cc1)NC(═O)Cc2cn3ccccc3[nH+]2 |
| C(CO[NH+]═C(N)N)[C@H](C(═O)[O—])[NH3+] | C1CN2[C@@H](C[NH2+]1)COC2═O |
| c1cc(ccc1C(═O)C[NH3+])Br | [H]/[N+]═C(\C)/N(CC)CC |
| C[N+](C)(C)C[C@@H](CC(═O)[O—])O | [H]/[N+]═C(/C)\N(CC)CC |
| c1ccc2c(c1)C[NH+]═C2N | C1CCN(CC1)C(═O)[C@H]2CCC[NH2+]C2 |
| Cn1cccc1C[NH2+]CCC2═C[N]c3c2cccc3 | CC[C@@](C)(C[NH3+])N1CCOCC1 |
| c1ccnc(c1)CNc2ccc3c(c2)[N]C(═O)[N]3 | c12c([nH]c(═O)[nH]c1═O)n[nH]n2 |
| Cn1cnc2c1c(═O)[nH]c(═O)n2C | c1cc(ccc1[C@H](C[NH3+])O)F |
| C1CC(CCC1C[NH3+])C(═O)[O—] | CC(C)c1nc(on1)[C@@H]2CCC[NH2+]2 |
| c1cc(c(cc1[C@H](C[NH3+])O)O)O | C[C@@H](C(═O)C1═C[N]c2c1cccc2)[NH+]3CCCCCC3 |
| C[C@H](C1CC1)[N@H+](C)Cc2[nH]c(═O)c3ccccc3n2 | C[C@@H](c1nc(nc(n1)N(C)C)N)[NH+]2CCCCCC2 |
| c1cc(cnc1)CC[NH3+] | c1cc(ccc1[C@@H](C[NH3+])O)F |
| c1cc(cc(c1)Cl)[C@H](C(═O)[O—])O | c1cc(cnc1)C(═O)N |
| C1CCN(C1)c2c(c([nH]n2)N)C#N | Cc1cccc(n1)NC(═O)C[N@](C)CC#N |
| C1COC[C@H]1C(═O)N | Cc1cc(no1)C(═O)NCC(F)(F)F |
| CC(C)C(═O)Nc1ccc(c(c1)C(F)(F)F)N(═O)═O | c1cscc1C(═[NH2+])N |
| CC(C)Nc\1ccccc/c1═N\C(C)C | CC(═O)N1C═Cc2ccccc2[C@@H]1CC(═O)N(C)C |
| C1COC[C@@H](C[NH2+]1)O | c1cc(oc1C[NH3+])C(F)(F)F |
| CC(═O)Nc1cccc(c1)C[NH3+] | C1CCC(CC1)C(═O)[N]C[C@@H]2CCCO2 |
| Cc1cc(ncn1)N2CCCCCC2 | c1cc(c(cc1N(═O)═O)O)[O—] |
| c1cc2c(cc1[C@H]3CCC[NH2+]3)OCCO2 | c1cc(ccc1C(═O)N)O |
| COC(═O)c1ccc(cc1)C[NH3+] | CN(C)c1cccc(c1)C(═O)NN |
| c1cc(cc(c1)Cl)[C@@H](C(═O)[O—])O | c1cc(c(cc1[C@@H]2CN3CC[NH+]═C3N═N2)F)F |
| Cn1cnc2c1c(═O)n(c(═O)n2C)C | Cc1cccc(c1)C(═O)NN |
| C[NH2+]Cc1ccc(o1)Oc2cccnc2 | CC1CCC(CC1)NC(═O)Cn2ccnc2 |
| C12C(NC(═O)N1)NC(═O)N2 | C1CC(═O)N[C@@H]1C(═O)[O—] |
| c1cc(cc(c1)N)C(═O)N | Cc1cc(no1)[N—]S(═O)(═O)c2ccc(cc2)N |
| C[C@@H](C1CC1)[N@H+](C)Cc2[nH]c(═O)c3ccccc3n2 | c1[nH]c2c(n1)C(═O)[N][C@@H](N2)[NH3+] |
| CC[C@](C)(C[NH3+])N1CCOCC1 | CN(C)NC(═O)CCC(═O)[O—] |
| C(C(═O)NCC(═O)NCC(═O)[O—])[NH3+] | C1CCC(CC1)C(═O)[N]C[C@H]2CCCO2 |
| c1[nH]c(═O)c2c(n1)[N]C(═[NH+]2)N3CCCCC3 | C1CN2[C@H](C[NH2+]1)COC2═O |
| CN(C)c1ccc(cn1)C(═O)[O—] | c1cc2c(cc1Cl)[nH]c(═O)o2 |
| c1cc(sc1S(═O)(═O)N)Cl | Cn1cc(nc1)C[C@@H](C(═O)[O—])[NH3+] |
| C1CC2(CCC1C2)C(═O)NO | C[NH+]Cc1nccn1C |
| Cc1c(c(n(n1)C)C)CC(═O)Nc2ccccn2 | c1cc(ccc1CC(═O)NN)F |
| C[C@H](C(═O)C1═C[N]c2c1cccc2)[NH+]3CCCCCC3 | c1cc2c(cc1NC(═O)C[NH+]3CCCCC3)OCO2 |
| COc1ccc(cc1O)C[NH3+] | |
| c1cc(ccc1C(═O)[O—])N(═O)═O | |

The 119 compound fragments were each docked into the active site of STAT6 and the top 40 poses for each were retained, resulting in a pose set consisting of 4,760 poses.

For purposes of visualization, in accordance with block 213, the pose set was clustered thereby assigning each pose in the pose set to a cluster in a plurality of clusters. This clustering was based off of spatial overlap between poses in the pose set by defining a two Angstrom radius around each atom and counting poses that had fewer than a 50% overlap of these spheres as separate. The plot of the lowest ranked pose (in terms of interaction energy between the compound fragment and the atomic model of STAT6) for each of these clusters is shown in FIG. 8. Note that the STAT6 model would be in front of the poses but the STAT6 model visibility has been toggled off for easier viewing in FIG. 8. The result, as illustrated in FIG. 8, is clusters of sulfurs (red), fluorines (green) and nitrogens (blue). However, the clearest feature is the large red group of oxygens towards the bottom of the structure (group 802). This is where interactions between poses in the pose set and LYS 544 of STAT6 take place.

In accordance with block 213, a corresponding subset of interaction features, drawn from a plurality of interaction features (Openeye interaction hints, three-dimensional partial charges, three-dimensional pharmacophores, etc.), was associated with each pose in the pose set. Each such interaction feature in the plurality of interaction features is associated with a corresponding subregion of the atomic model of the target macromolecule. Openeye interactions hints are interactions perceived by the OEPerceiveInteractionHints function (OpenEye Scientific/Cadence Molecular Systems Interactions, Santa Fe, New Mexico).

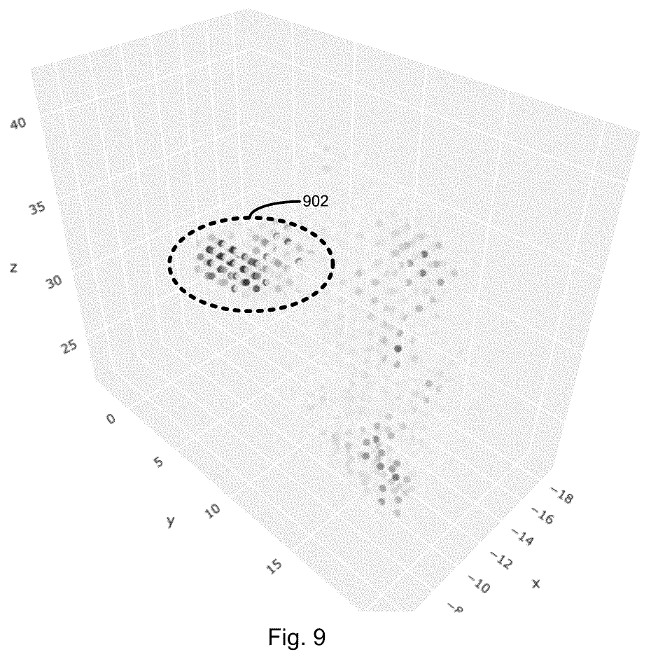

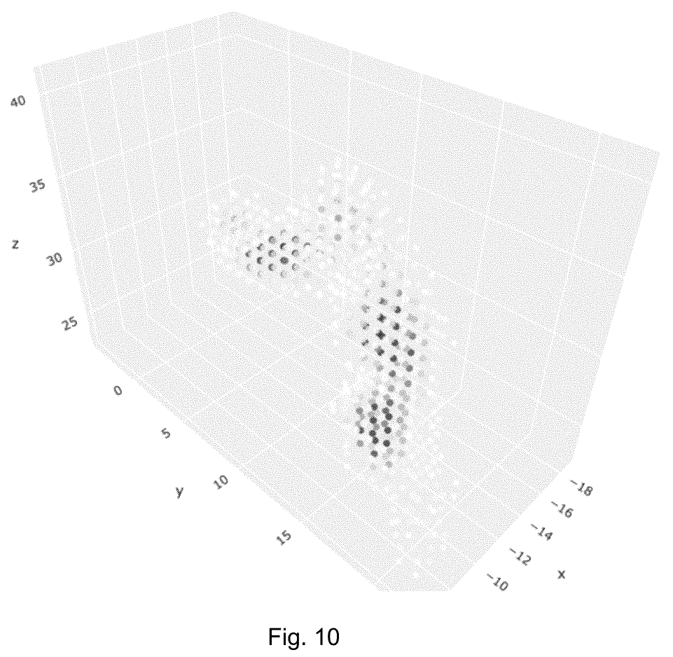

Further in this example, the aggregation of partial charges (FIG. 9) and pharmacophores (FIG. 10) was viewed. To generate FIGS. 9 and 10, the mean across all poses of all fragments in the pose set was determined. In FIGS. 9 and 10, the model is rotated approximately 90 degrees clockwise and out of the page as compared to FIG. 8. The red charges (element 902) in FIG. 9 align with the red atoms (element 802) in FIG. 8. The scales have been adjusted in FIGS. 9 and 10 to show variability and any grid points with a mean absolute value less than 0.001 have been removed.

The largest hydrophobic regions are the region just above the LYS 544 interactions (as viewed in FIG. 8), element 804, and the hydrophobic pocket (top right of FIG. 8), element 806 with a bit at the top middle of FIG. 8 (element 808).

The most common OpenEye interaction hints as well as their frequency across all poses and fragments is listed in Table 3.

TABLE 3
most common interaction hints.

| Residue | Interaction type | Frequency |
|---|---|---|
| LYS 544 | cationpi:ligandpi | 0.211298 |
| LYS 544 | salt-bridge:ligand−protein+ | 0.180288 |
| SER 566 | hbond:protein2ligand | 0.133173 |
| ARG 562 | salt-bridge:ligand−protein+ | 0.126683 |
| GLU 651 | salt-bridge:ligand+protein− | 0.119231 |

The top partial charge interactions are listed in Table 4.

TABLE 4
top partial charge interactions, where “Mean PC3” stands
for “Mean partial charge 3-Dimensional”

| Coordinate | | Residue | Residue |
|---|---|---|---|
| name | Mean PC3 | name/atom | Distance |
| 3006 (−13.0, 2.0, 29.0) | 0.042212 | SER 564 OG | 3.260039 |
| 2918 (−14.0, 1.0, 29.0) | 0.033454 | LYS 544 NZ | 3.500049 |
| 3001 (−13.0, 1.0, 29.0) | 0.019848 | ARG 562 NH1 | 3.298422 |
| 2914 (−14.0, 0.0, 29.0) | 0.017729 | LYS 544 NZ | 3.75797 |
| 1616 (−11.0, 17.0, 38.0) | 0.01536 | GLU 651 OE2 | 1.263087 |
| 2924 (−14.0, 2.0, 29.0) | 0.01483 | SER 566 OG | 3.368016 |
| 1615 (−11.0, 17.0, 37.0) | 0.01451 | GLU 651 OE2 | 1.890871 |
| 3014 (−13.0, 3.0, 30.0) | 0.012607 | THR 572 CG2 | 3.737348 |
| 3005 (−13.0, 2.0, 28.0) | 0.011088 | SER 564 OG | 2.313839 |
| 771 (−10.0, 17.0, 25.0) | 0.010991 | ARG 605 NH1 | 3.711912 |
| 3013 (−13.0, 3.0, 29.0) | 0.010821 | SER 564 OG | 3.156557 |
| 1625 (−11.0, 18.0, 37.0) | 0.01047 | GLU 651 OE2 | 1.500463 |
| 1717 (−10.0, 17.0, 38.0) | 0.010412 | GLU 651 OE2 | 1.654505 |
| 3076 (−12.0, 1.0, 29.9) | −0.046156 | ARG 562 NH1 | 2.538028 |
| 3077 (−12.0, 1.0, 30.0) | −0.045205 | ARG 562 NH1 | 2.020294 |
| 3611 (−12.0, 0.0, 29.0) | −0.045008 | ARG 562 NH2 | 2.279337 |
| 2916 (−14.0, 1.0, 27.0) | −0.040345 | SER 566 N | 2.427199 |
| 2917 (−14.0, 1.0, 28.0) | −0.031641 | SER 566 OG | 2.888517 |
| 2923 (−14.0, 2.0, 28.0) | −0.030031 | SER 564 OG | 2.640048 |
| 3082 (−12.0, 2.0, 30.0) | −0.029933 | ARG 562 NH1 | 2.590673 |
| 3612 (−12.0, 0.0, 30.0) | −0.026545 | ARG 562 NH1 | 1.857844 |
| 3592 (−13.0, 0.0, 29.0) | −0.024652 | ARG 562 NH2 | 3.092147 |
| 3002 (−13.0, 1.0, 30.0) | −0.023533 | ARG 562 NH1 | 2.918833 |
| 3593 (−13.0, 0.0, 30.0) | −0.023136 | ARG 562 NH1 | 2.808841 |
| 2930 (−14.0, 3.0, 28.0) | −0.020328 | SER 564 OG | 2.511146 |
| 2922 (−14.0, 2.0, 27.0) | −0.016739 | SER 564 OG | 1.92246 |
| 2817 (−15.0, 1.0, 28.0) | −0.016657 | SER 566 OG | 2.213036 |
| 3597 (−13.0, 1.0, 27.0) | −0.016363 | SER 564 OG | 2.178039 |
| 2840 (−15.0, 4.0, 31.0) | −0.016181 | LYS 544 NZ | 2.865369 |
| 3765 (−14.0, 0.0, 27.0) | −0.016054 | SER 566 N | 2.151115 |
| 2847 (−15.0, 5.0, 30.0) | −0.015837 | PRO 591 CD | 4.099616 |
| 3097 (−12.0, 4.0, 31.0) | −0.015665 | THR 572 OG1 | 2.535941 |
| 2824 (−15.0, 2.0, 29.0) | −0.01563 | SER 566 OG | 2.810255 |

The top hydrophobic interactions are listed in Table 5.

TABLE 5
top hydrophobic interactions, where “Mean Ph3” stands
for “Mean pharmacophore 3-Dimensional”

| Coordinate | Mean Ph3 | Residue | Residue |
|---|---|---|---|
| name | hydrophobe | name/atom | Distance |
| 1244 (−14.0, 4.0, 30.0) | 0.127748 | LYS 544 NZ | 3.718648 |
| 1251 (−14.0, 5.0, 30.0) | 0.104375 | PRO 591 CD | 3.525175 |
| 280 (−11.0, 15.0, 26.0) | 0.095139 | PHE 592 CE1 | 3.890506 |
| 1186 (−15.0, 5.0, 30.0) | 0.088292 | PRO 591 CD | 4.099616 |
| 654 (−11.0, 16.0, 32.0) | 0.0798 | MET 648 SD | 4.095525 |
| 655 (−11.0, 16.0, 33.0) | 0.078304 | MET 648 SD | 4.597752 |
| 288 (−11.0, 16.0, 26.0) | 0.076077 | ARG 605 NH1 | 4.346986 |
| 1302 (−13.0, 4.0, 30.0) | 0.066928 | THR 572 OG1 | 3.621187 |
| 711 (−10.0, 16.0, 33.0) | 0.066028 | MET 648 SD | 3.831883 |
| 1237 (−14.0, 3.0, 30.0) | 0.065741 | LYS 544 NZ | 3.114537 |
| 1252 (−14.0, 5.0, 31.0) | 0.06536 | ASN 588 CB | 4.018299 |
| 1194 (−15.0, 6.0, 30.0) | 0.064559 | PRO 591 CD | 3.618681 |
| 1178 (−15.0, 4.0, 30.0) | 0.063371 | LYS 544 NZ | 3.319087 |
| 279 (−11.0, 15.0, 25.0) | 0.063132 | ASP 596 OD1 | 3.846709 |
| 1245 (−14.0, 4.0, 31.0) | 0.062233 | LYS 544 NZ | 3.319991 |
| 645 (−11.0, 15.0, 33.0) | 0.06152 | MET 648 SD | 4.749034 |
| 289 (−11.0, 16.0, 27.0) | 0.060136 | PHE 592 CE1 | 4.161735 |
| 701 (−10.0, 15.0, 33.0) | 0.058628 | MET 648 SD | 4.012148 |
| 281 (−11.0, 15.0, 27.0) | 0.057931 | PHE 592 CE1 | 3.494287 |
| 644 (−11.0, 15.0, 32.0) | 0.057601 | MET 648 SD | 4.26466 |

From this example it is seen that, for stat6, the virtual fragment soaking was able to find the most important regions, as well as highlight potential other regions of interest. This can be converted into a hypothesis that can be used to refine manual and/or computational hypotheses. Such computational hypotheses can be used for design space reduction (DSR) runs or MolGen runs.

In fact, the data can be used to develop several hypotheses, each with a subset of the interaction features identified in this example, to empirically through DSR, in accordance with block 270), or generated using MolGen.

The present example shows promise of getting around a common problem with docking: false positives. First, a fragment will bind wherever it can, so there aren't really “false positives” since it is likely every fragment can bind somewhere in a pocket. Comparing this to larger drug-like compounds that will be forced to find a pose, even if the interactions are weak. Second, individual fragment's poses are not of interest, what is of interest is the aggregate poses of the compound fragments. This means that it doesn't matter if half the compound fragments have a useless conformation, if the important parts making the interactions are fairly consistent.

The advantage of the virtual fragment screen as illustrated in this example is that dozens of compound fragments are used and only regions where they cluster together are considered. Moreover, exact compound fragment poses are not needed, all that is needed is hints of where these compound fragments may interact. This information can be used to inform downstream DSR and MolGen frameworks, to build out diverse compounds that can test these hypotheses. With just a few consciously chosen representative compound fragments, “important” compound fragment region can be validated and the target macromolecular binding hypotheses 180 updated accordingly.

This example demonstrates an improved approach over conventional fragment-based drug design. Rather than linking disparate compound fragments, or merging them, or using their profile to screen larger databases, the present disclosure abstract the overlay of many compound fragments into their charge and pharmacophore profiles and, from this generates diverse chemical matter that can match these profiles. This helps solve the “cold-start” problem in drug discovery. For instance, in accordance with block 232, each respective pose in the plurality of poses can be quantified by applying a physics model to each respective pose in the pose set, thereby assigning a score to each interaction feature in the plurality of interaction features. With the interaction features quantified, a target macromolecule binding hypothesis can be constructed using the plurality of interaction features and the score assigned to each interaction feature in the plurality of interaction features in accordance with block 244. Alternatively the target macromolecule binding hypothesis can be obtained by selecting interaction features from FIGS. 8-10, or Tables 3-5. The target macromolecule binding hypothesis can then be used to identify a plurality of derived compounds in accordance with block 256. The plurality of derived compounds is then tested for activity against the target macromolecule in accordance with block 272.

### CONCLUSION

The foregoing description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

1. A method for identifying one or more compounds that exhibit a threshold activity with respect to a target macromolecule, the method comprising:
A) generating, using a computer, for each respective compound fragment in a plurality of compound fragments, a corresponding plurality of poses of the respective compound fragment against an atomic model of the target macromolecule thereby constructing a pose set;
B) associating, using a computer, a corresponding subset of interaction features, drawn from a plurality of interaction features, to each pose in the pose set, wherein each interaction feature in the plurality of interaction features is associated with a corresponding subregion of the atomic model of the target macromolecule;
C) quantifying, using a computer, each respective pose in the plurality of poses by applying a physics model to each respective pose in the pose set, thereby assigning a score to each interaction feature in the plurality of interaction features;
D) forming, using a computer, a target macromolecule binding hypothesis using the plurality of interaction features and the score assigned to each interaction feature in the plurality of interaction features;
E) identifying a plurality of derived compounds based on the target macromolecule binding hypothesis; and
F) testing the plurality of derived compounds for activity against the target macromolecule, thereby identifying one or more compounds that exhibit the threshold activity with respect to the target macromolecule.

2. The method of claim 1, wherein the atomic model comprises a plurality of residues and the corresponding subregion of the atomic model is a first residue in the plurality of residues or an atom of the first residue.

3. The method of claim 1, wherein the atomic model comprises a plurality of residues and the corresponding subregion of the atomic model is a subset of residues in the plurality of residues or a plurality of atoms of the first subset of residues.

4. The method of claim 1, wherein the corresponding subregion of the atomic model comprises a portion of a surface of the atomic model of the target macromolecule.

5. The method of claim 1, wherein the corresponding subregion of the atomic model comprises a portion of a surface of the atomic model of the target macromolecule.

6. The method of claim 1, wherein the physics model is a first model comprising a first plurality of parameters and the quantifying C) comprises:
inputting the respective pose into the first model, and
obtaining, through application of the respective pose to the first plurality of parameters in accordance with the first model, a pose score for the respective pose; and the method further comprises
using the pose score for each respective pose in the plurality of poses to determine a score for each interaction feature in the plurality of interaction features.

7. The method of claim 1, wherein the physics model evaluates an interaction energy of the pose.

8. The method of claim 7, wherein the physics model evaluates the interaction energy of the pose using a calculated potential energy surface of the pose.

9-10. (canceled)

11. The method of claim 1, wherein the physics model evaluates the pose against an interaction feature contract.

12. The method of claim 1, wherein the target macromolecule binding hypothesis comprises a top N interaction features in the plurality of interaction features, wherein N is a positive integer.

13-24. (canceled)

25. The method of claim 1, wherein the threshold activity with respect to the target macromolecule is an IC₅₀, EC₅₀, Kd, KI, hill coefficient (nH), negative logarithm of EC₅₀ (pEC₅₀), association rate constant (Kon), or disassociation rate constant (Koff), for a compound with respect to the target macromolecule.

26. The method of claim 1, wherein the plurality of interaction features collectively identifies between 30 and 700 atoms of the target macromolecule.

27. (canceled)

28. The method of claim 1, wherein the forming D) includes identifying a set of residues of the target macromolecule that are included in the target macromolecule binding hypothesis.

29-31. (canceled)

32. The method of claim 1, wherein the forming D) comprises:
selecting a subset of poses from the plurality of poses that each has an interaction feature associated with a first residue in a plurality of residues of the atomic model;
selecting a first pose from the subset of poses that has a lowest energy score in the subset of poses; and
including one or more interaction features associated with the first pose in the target macromolecule binding hypothesis.

33. The method of claim 32, wherein the forming D) further comprises:
selecting a second pose from the plurality of poses on the basis that it is associated with a second interaction feature that is other than any of the one or more interaction features associated with the first pose; and
including the second interaction feature in the target macromolecule binding hypothesis.

34. (canceled)

35. The method of claim 1, wherein the identifying E) comprises generating the plurality of derived compounds constrained to the target macromolecule binding hypothesis.

36. The method of claim 1, wherein the identifying E) comprises:
generating a plurality of initial compounds using the target macromolecule binding hypothesis; and
evolving at least a subset of the plurality of initial compounds into the plurality of derived compounds using a reinforcement learning process.

37. The method of claim 36, wherein the reinforcement learning process eliminates at least a subset of the plurality of initial compounds.

38. The method of claim 36, wherein the reinforcement learning process comprises:
i) generating, using a computer, a plurality of experiences, each respective experience in the plurality of experiences using an initial compound selected from the plurality of initial compounds to construct a corresponding derived compound in the plurality of derived compounds through a hierarchical proximal policy comprising a parent model and a child model using an environment of the target macromolecule, wherein
the parent model is a molecular reaction model that evaluates a plurality of molecular reactions to apply to an initial compound to form the derived compound, and
the child model is a reactant model that evaluates a corresponding plurality of reactants for a molecular reaction applied to the initial compound to form the derived compound.

39. The method of claim 38, wherein the reinforcement learning process further comprises:
ii) updating, using a computer, a second plurality of parameters associated with the parent model in accordance with a first surrogate objective calculated using the plurality of experiences;
iii) updating, using a computer, a third plurality of parameters associated with a child model in accordance with a second surrogate objective using the plurality of experiences;
iv) repeating, using a computer, the generating i), updating ii), and updating iii) until a threshold convergence criterion is satisfied.

40. The method claim 38, wherein an experience in the plurality of experiences is generated by:
(a) initializing the experience to state t 0,
(b) inputting a complex of state t, in two or three dimensions, of the initial compound in state t interacting with the environment of the target macromolecule into the parent model, wherein the parent model evaluates, using a computer, a first exit vector of the initial compound in state t against the plurality of molecular reactions, thereby assigning a corresponding probability to each respective molecular reaction in the plurality of molecular reactions for state t,
(c) selecting a molecular reaction in the plurality of molecular reactions, using a computer, through a sampling of the plurality of molecular reactions using the corresponding probability assigned to each molecular reaction in the plurality of molecular reactions for state t,
(d) inputting the complex of state t into the child model, wherein the child model evaluates, using a computer, the initial compound in state t against each reactant in a corresponding plurality of reactants available for reaction using the molecular reaction selected for state t, thereby assigning a corresponding probability to each respective reactant in the corresponding plurality of reactants for state t,
(e) selecting, using a computer, a reactant in the corresponding plurality of reactants, through a sampling of the corresponding plurality of reactants using the corresponding probability assigned to each reactant in the corresponding plurality of reactants for state t,
(f) advancing state t to state t+1,
(g) forming, using a computer, the initial compound in state t through an in silico reaction of the initial compound in state t−1 in accordance with the selected molecular reaction and the selected reactant of state t,
(h) determining a score, using a computer, for the initial compound in state t interacting with the environment of the target macromolecule by inputting the initial compound in state t interacting with the environment of the target macromolecule into a physics model, and
(i) repeating the (b) inputting, (c) selecting, (d) inputting, (e) selecting, (f) advancing, (g) forming, and (h) determining until a compound exit criterion is satisfied by the initial compound in state t, thereby forming a plurality of states for the experience.

41. The method of claim 1, wherein the testing F) tests the plurality of derived compounds using a quantum mechanics algorithm.

42. The method of claim 1, wherein the testing F) tests the plurality of derived compounds using a wet lab assay.

43. The method of claim 1, wherein the identifying E) comprises in silico screening of a database of compounds using the on the target macromolecule binding hypothesis as a selection criterion.

44. A computer system for identifying one or more compounds that exhibit a threshold activity with respect to a target macromolecule, the computer system comprising:
one or more processors; and
memory addressable by the one or more processors, the memory storing at least one program for execution by the one or more processors, the at least one program comprising instructions for:
A) generating, for each respective compound fragment in a plurality of compound fragments, a corresponding plurality of poses of the respective compound fragment against an atomic model of the target macromolecule thereby constructing a pose set;
B) associating a corresponding subset of interaction features, drawn from a plurality of interaction features, to each pose in the pose set, wherein each interaction feature in the plurality of interaction features is associated with a corresponding subregion of the atomic model of the target macromolecule;
C) quantifying each respective pose in the plurality of poses by applying a physics model to each respective pose in the pose set, thereby assigning a score to each interaction feature in the plurality of interaction features;
D) forming a target macromolecule binding hypothesis using the plurality of interaction features and the score assigned to each interaction feature in the plurality of interaction features;
E) identifying a plurality of derived compounds based on the target macromolecule binding hypothesis; and
F) testing the plurality of derived compounds for activity against the target macromolecule, thereby identifying one or more compounds that exhibit the threshold activity with respect to the target macromolecule.

45. A non-transitory computer readable storage medium, wherein the non-transitory computer readable storage medium stores instructions, which when executed by a computer system, cause the computer system to perform a method of identifying one or more compounds that exhibit a threshold activity with respect to a target macromolecule, the method comprising:
A) generating, for each respective compound fragment in a plurality of compound fragments, a corresponding plurality of poses of the respective compound fragment against a atomic model of the target macromolecule thereby constructing a pose set;
B) associating a corresponding subset of interaction features, drawn from a plurality of interaction features, to each pose in the pose set, wherein each interaction feature in the plurality of interaction features is associated with a corresponding subregion of the atomic model of the target macromolecule;
C) quantifying each respective pose in the plurality of poses by applying a physics model to each respective pose in the pose set, thereby assigning a score to each interaction feature in the plurality of interaction features;
D) forming a target macromolecule binding hypothesis using the plurality of interaction features and the score assigned to each interaction feature in the plurality of interaction features;
E) identifying a plurality of derived compounds based on the target macromolecule binding hypothesis; and
F) testing the plurality of derived compounds for activity against the target macromolecule, thereby identifying one or more compounds that exhibit the threshold activity with respect to the target macromolecule.

46-112. (canceled)
